(19) United States
(12) Patent Application Publication
(10) Pub. No.: US 2022/0089600 A1
(43) Pub. Date: Mar. 24, 2022

(54) TGFß INHIBITOR AND PRODRUGS

(71) Applicants: FUNDACIÓ INSTITUT DE RECERCA BIOMÈDICA (IRB BARCELONA), Barcelona (ES); INSTITUCIÓ CATALANA DE RECERCA I ESTUDIS AVANÇATS, Barcelona (ES); UNIVERSITAT DE BARCELONA, Barcelona (ES)

(72) Inventors: Daniele TAURIELLO, Barcelona (ES); Daniel BYROM, Barcelona (ES); Joan Antoni MATARÍN MORALES, Barcelona (ES); Eduard BATLLE GÓMEZ, Barcelona (ES); Antoni RIERA ESCALÉ, Barcelona (ES)

(21) Appl. No.: 17/296,329

(22) PCT Filed: Nov. 22, 2019
(86) PCT No.: PCT/EP2019/082215

§ 371 Date: May 24, 2021

(30) Foreign Application Priority Data
Nov. 22, 2018 (EP) 18382843.3

(51) Int. Cl. C07D 487/04; A61K 45/06; A61P 35/04

(52) U.S. Cl. CPC C07D 487/04; A61P 35/04; A61K 45/06

(57) ABSTRACT

A compound of formula I

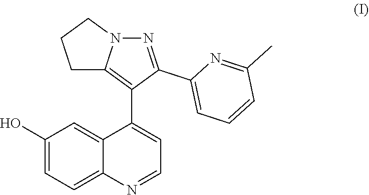 [SMILES: Cc1cccc(-c2nn3c(c2-c2ccnc4ccc(O)cc24)CCC3)n1]

with improved TGFβ signaling pathway inhibitory activity, improved therapeutic efficacy and improved toxicity profile, as well as two prodrugs thereof are disclosed.

Compositions comprising said TGFβ signaling pathway inhibitor and prodrugs thereof are also disclosed. Additionally, present invention discloses said compound of formula I and prodrugs thereof for use in a method of treating diseases responsive to TGFβ signaling pathway inhibition.

### FIELD OF THE INVENTION

Present invention refers to a novel TGFβ signaling pathway inhibitor with improved inhibitory activity, improved therapeutic capacity and improved toxicity profile, as well as to prodrugs thereof, comprising an acetovanillone-derived fragment or a carbamate fragment, which cage the TGFβ signalling inhibiting activity but, when said prodrugs are hydrolysed are able to deliver the more active TGFβ signaling pathway inhibitor in vivo. Compositions comprising said TGFβ signaling pathway inhibitor and prodrugs thereof are also disclosed. Further, present invention discloses said TGFβ signaling pathway inhibitor and prodrugs thereof for use in a method of treating diseases responsive to TGFβ signaling inhibition.

### BACKGROUND OF THE INVENTION

Transforming growth factor beta isoforms, TGFβ1, TGFβ2, and TGFβ3, are small secreted homodimeric signaling proteins. They are present only in vertebrates and are required for the proper development as well as homeostasis of several organs and tissues. Most, if not all, of the activities in regulating cellular fates and functions of the TGFβ signaling pathway are mediated by specific receptor complexes that are assembled upon ligand binding and comprise TGFβ type II receptors and type I receptors.

TGFβ receptors (TGFβR) include TGFβ type I receptors and TGFβ type II receptors. TGFβ type I receptors include seven Activin receptor-Like Kinase: ALK1 (ACVRL1), ALK2 (ACVR1), ALK3 (BMPR1A), ALK4 (ACVR1B), ALK5 (TGFBR1), ALK6 (BMPR1B) and ALK7 (ACVR1C), and TGFβ type II receptors include TGFBR2, BMPR2, ACVR2A, and ACVR2B. TGFβ ligands use ALK5/TGFBR1 and TGFBR2 to signal across the cell membrane and are therefore targets for specific inhibition of the TGFβ pathway. Especially ALK5 has been used as a target with small molecules, but because receptors ALK4 and ALK5 are closely related, inhibitor specificity (for either of them) is a known problem. ALK4/ACVR1B mediates signaling from additional molecules in the TGFβ superfamily, including Activin A, GDF1, GDF11, and Nodal. However, similar to ALK5 signaling, ALK4 activity has been involved in tissue fibrosis (Jin., et al., Journal of Mecinal Chemistry 2014, 57: 4213-4238; Sugiyama M et al., Gastroenterology 1998, 114(3): 550-558; Matsuse T et al., Am. J. Respir. Cell Mol. Biol. 1995, 13(1): 17-24; Pawlowski, J. E. et al., J. Clin. Invest. 1997, 100(3):639-648; Hübner, G. et al, Dev. Biol. 1996, 173(2): 490-498; Munz, B. et al., EMBO J. 1999,18(19): 5205-5215; Wankell, M. et al, EMBO J. 2001, 20(19): 5361-5372. Therefore, dual inhibition of ALK4/ALK5 might enhance the therapeutic efficacy of TGFβ pathway inhibitors in fibrotic diseases.

TGFβ signaling has been implicated in a wide range of pathologies, including genetic disorders (including Camurati-Engelmann disease, Marfan syndrome, muscular dystrophy, and Fanconi Anemia), obesity, diabetes, hematological diseases, cardiovascular diseases, skin diseases such as psoriasis and fibrotic diseases, and plays a key role in wound healing or scarring of e.g. burn wounds.

TGFβ signaling also plays a key role in cancer progression. While TGFβ signaling potentially affects all different cell types in tumors in complex and disparate ways, the overall effect seems to strongly promote tumor growth, invasion and metastasis, particularly at late stages of the disease. Therefore, inhibition of TGFβ signaling pathway is strongly relevant in the clinic, and the development and testing of effective agents that target this pathway is being actively pursued. Clinical trials are ongoing for a variety of cancer types, including hematologic cancers such as multiple myeloma and myelodysplastic syndrome; brain cancers such as glioblastoma; soft cancers such as Ewing's sarcoma and malignant pleural mesotheliomas; and solid tumors including breast cancer, gastric and gastroesophageal cancer, colorectal cancer, pancreatic cancer, liver cancer, lung cancer, ovarian cancer, prostate cancer, squamous cell carcinoma or melanoma; as well as metastases related to the mentioned cancer types.

Documents Li et al. (J. Med. Chem. 2008, 51(7): 2302-2306), WO02094833 A1 and WO2014072517 A1 describe a wide range of dihydropyrrolopyrazole derivatives for use as TGFβ signaling pathway inhibitors.

Recently, it has been shown that TGFβ signaling inhibition activates anti-tumor immune responses and potentiates other types of immunotherapies, making it a likely component of combinatorial immunotherapy in the near future for treatment of different types of cancers (Batlle and Massagué. Immunity. 2019, 50(4): 924-940). It has been shown that inhibition of stromal TGFβ signaling unleashes the immune system against the tumor cells and this therapeutic effect also cures metastatic disease, the main cause of death in cancer patients (Tauriello et al. Nature. 2018, 554(7693): 538-543.; Mariathasan et al. Nature. 2018, 554(7693):544-548).

Although it is widely recognized as a potential therapeutic target, clinical exploitation of the TGFβ pathway has been hampered by important toxicity issues. The development of several small molecule inhibitors and antibodies directed against pathway components has been abandoned due to their extensive toxicity in preclinical models and patients (Garber, K. (2009). JNCI Journal of the National Cancer Institute, 101(24), 1664-1667.). The small molecule LY2157299 (galunisertib) is a TGFβ pathway inhibitor that has been extensively tested in cancer patients. Galunisertib is a relatively weak inhibitor of TGFβ receptor I (ALK5) (Herbertz S et al., Drug Design, Development and Therapy, 2015, 9: 4479-4499) that entered clinical trials, initially for brain cancer and, over the past 10 years, has been tested in multiple other cancer types (Herbertz S et al., Drug Design, Development and Therapy, 2015, 9: 4479-4499)). Given its associated toxicity in preclinical models, including oral cardiovascular, gastrointestinal, immune, bone/cartilage, reproductive, and renal toxicity (Stauber et al., J Clin Pract 2014, 4(3): 1-10), both the dosing and the frequency of administration of Galunisertib has been limited to a maximum of 300 mg/day in humans in a regime of 14 days on-14 days off (Herbertz S et al., Drug Design, Development and Therapy, 2015, 9: 4479-4499). However, despite extensive testing in clinical trials, the therapeutic effects of galunisertib in cancer patients reported so far using this dose regime have been modest or non-existent (Herbertz S et al; 2015; Melisi et al., British Journal of Cancer, 2018, 119(10): 1208-1214; Kelley R K, et al., Clin Transl Gastroenterol. 2019 July; 10(7):e00056.; Brandes, A. et al., Neuro-Oncology, 18(8): 1146-1156.). Therefore, inhibitors of the TGFβ signaling pathway that exhibit lower toxicity profiles and enhanced activity at therapeutic doses represent an important clinical need.

More in general, it is known that many effective drugs provide numerous adverse effects or are toxic to healthy tissues and cells. Others can only be delivered locally to avoid systemic effects of the drug. Prodrugs are compounds deriving from active medicaments or drugs (parent compound), which have been modified to modulate properties such as solubility, absorption, distribution, metabolism, excretion and toxicity. Therefore, prodrugs are compounds which include the parent compound attached to a fragment which reduces or blocks the activity, whilst at the same time modulating one or more properties such as solubility, absorption, distribution, metabolism, excretion and toxicity. In this sense Yoon et al. (Molecular Cancer Therapeutics, 2003, 2(11): 1171-1181) report the use of an ester-linked 4-piperidinopiperidine group as a prodrug of the active metabolite 7-ethyl-10-hydroxycamptothecin. Additionally, Reiter et al. (Green Chem., 2013, 15 (5): 1373-1381) report the enzymatic cleavage of lignin β-4 aryl ether bonds with a LigF (β-etherase) enzyme.

Accordingly, one objective of present invention is developing a more effective TGFβ signaling pathway inhibitor that exhibit an improved toxicity profile and enhanced therapeutic effects.

Additionally, it is also the objective of present invention to develop prodrugs which can be used to deliver a drug more efficiently, in the target tissue or organ, avoiding thus adverse or non-desired systemic effects: creating highly efficient local doses, while sparing the rest of the body of toxicity.

### BRIEF DESCRIPTION OF THE INVENTION

Present invention refers to a compound of formula (I), or a pharmaceutically acceptable salt, or a pharmaceutically acceptable solvate thereof:

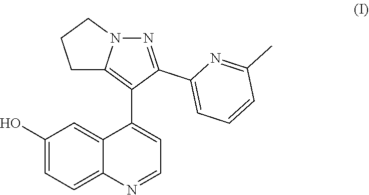 [SMILES: Cc1cccc(-c2nn3c(c2-c2ccnc4ccc(O)cc24)CCC3)n1]

Present invention also relates to a pharmaceutical composition comprising an effective amount of the compound of formula I, or a pharmaceutically acceptable salt, or a pharmaceutically acceptable solvate thereof, and/or at least one pharmaceutical acceptable excipient or carrier and, optionally, at least another active ingredient.

Additionally, present invention refers to a compound of formula I or a pharmaceutically acceptable salt, or a pharmaceutically acceptable solvate thereof, or a pharmaceutical composition comprising said compound of formula I or a pharmaceutically acceptable salt, or a pharmaceutically acceptable solvate thereof, for use as a medicament, in particular for use in the treatment of a disease responsive to inhibitors of the TGFβ pathway.

Present invention also relates to a prodrug of the compound of formula I, wherein said prodrug is a prodrug of formula (II) or a pharmaceutical salt, or a pharmaceutically acceptable solvate thereof:

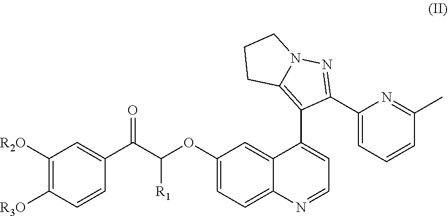

wherein
R₁ is selected from the group consisting of H, alkyl, cycloalkyl, haloalkyl, hydroxyalkyl, aminoalkyl, alkanoylamino, alkoxy, aryl, alkylaryl, arylalkyl, amino, N-alkylamino and N,N-dialkylamino; and
R₂ and R₃ are each independently selected from the group consisting of H, alkyl and haloalkyl.

Present invention also relates to a prodrug of the compound of formula I, said prodrug having formula (III) or a pharmaceutical salt, or a pharmaceutically acceptable solvate thereof:

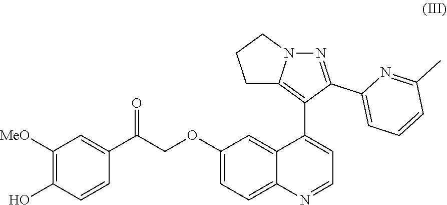 [SMILES: COc1cc(C(=O)COc2ccc3nccc(-c4c(-c5cccc(C)n5)nn5c4CCC5)c3c2)ccc1O]

Further, present invention relates to a prodrug of the compound of formula I, said prodrug having formula (IV) or a pharmaceutical salt, or a pharmaceutically acceptable solvate thereof:

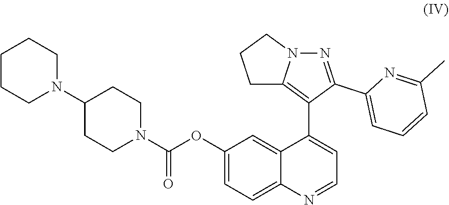 [SMILES: Cc1cccc(-c2nn3c(c2-c2ccnc4ccc(OC(=O)N5CCC(N6CCCCC6)CC5)cc24)CCC3)n1]

Further, present invention also refers to a pharmaceutical composition comprising an effective amount a prodrug of a compound of formula I, selected from a prodrug of formula II or a prodrug of formula III or a prodrug of formula IV, or a pharmaceutical salt, or a pharmaceutically acceptable solvate thereof, and at least one pharmaceutical acceptable excipient or carrier.

Present invention also refers to a prodrug of a compound of formula I, selected from a prodrug of formula II or a prodrug of formula III or a prodrug of formula IV, or a pharmaceutical salt, or a pharmaceutically acceptable solvate thereof, or a pharmaceutical composition comprising said prodrug, or a pharmaceutical salt, or a pharmaceutically acceptable solvate for use as a medicament, in particular for use in the treatment of diseases responsive to inhibitors of the TGFβ pathway. In particular for use in a disease responsive to the inhibition of the TGFβ signaling pathway selected from the group consisting of cancer, scleroderma, psoriasis, anemia, sarcopenia, Alzheimer's disease, Marfan syndrome, aneurysm, pulmonary hypertension, osteogenesis imperfecta, idiopathic pulmonary fibrosis, liver fibrosis, cirrhosis, hepatic steatosis, hypertrophic cardiomyopathy, myelofibrosis, neurofibromatosis type I, fibrotic kidney disease, focal segmental glomerulosclerosis, radiation-induced fibrosis, skin fibrosis in systemic sclerosis, diffuse systemic sclerosis, scarring, corneal primary pterygium, fibrosis, uterine leiomyoma, obesity, diabetes, microangiopathy in diabetic retinopathy and nephropathy, and more in particular for use in the treatment of cancer.

### BRIEF DESCRIPTION OF THE FIGURES

FIG. 1: IC₅₀ values obtained in a cellular assay with a CAGA-Luciferase reporter. Bioluminiscence (y axis) at different dosages of galunisertib [symbol] )), of the compound of formula I ( [symbol] ), the compound of formula III ( [symbol] ) and of the compound of formula IV ( [symbol] ). The calculated IC₅₀ values are shown in the legend.

FIG. 2: Photographs of liver metastasis sections, stained by immunohistochemistry for phosphorylated SMAD2: arrows point to stained cellular nuclei, indicating active TGFβ signaling in the control (vehicle) but not in mice treated with compounds of formula I, IV or galunisertib. Equimolar doses (5×) of galunisertib, compound of formula I and compound of formula IV were given during three days.

FIG. 3: Mouse liver metastasis initiation assay as a readout for TGFβ inhibition and associated anti-cancer efficacy with immunocompetent mice implanted with Mouse Tumor Organoids (MTOs). The number of liver metastases (LiMs) was measured (y axis) after treatment with the compound of formula I or galunisertib at a molar equivalent of 0.3×, 1×, 3× and 9× of the standard mouse dose of 80 mg/kg b.i.d. of galunisertib (which translates to 2 mg per mouse, assuming an average weight of 25 g), versus a control that was treated with empty vehicle. The dose of 0.3× galunisertib (0.6 mg/mouse b.i.d.) was not tested (n.t.)

FIG. 4: Mouse liver metastasis initiation assay as a readout for TGFβ inhibition and associated anti-cancer efficacy. Normalized bioluminescence signal, quantified from intravital imaging performed with luciferase-expressing MTOs implanted in immunocompetent mice shows tumor growth and rejection upon treatment with the compound of formula I and galunisertib at different doses (0.3×, 1× and 3× the molar equivalent of the standard mouse galunisertib dose of 80 mg/kg b.i.d.) between days 3 and 14.

FIG. 5: In vitro cytotoxicity assays of the compound of formula I in HEK293T cells versus that of galunisertib. DMSO was used as a control.

FIG. 6: In vitro fluorescence assays with purified LigF for enzymatic cleavage of the β-keto ether moiety of 4-methylumbelliferone acetovanillone, model compound of the prodrug of formula II, and in particular of the prodrug of formula Ill. Figure shows fluorescence versus time.

FIG. 7: In vitro fluorescence assays with HEK293T cells stably expressing LigF for cellular enzymatic cleavage of the β-keto ether moiety of 4-methylumbelliferone acetovanillone (MUAV), model compound of the prodrug of formula II, and in particular of the prodrug of formula Ill. Figure shows fluorescence of different concentrations of MUAV, exposed to LigF versus time. Three concentrations of MUAV substrate are indicated: 10 μM (filled square), 30 μM (triangle) and 100 μM (filled circle). Control with 100 μM MUAV but no LigF is shown with empty circles.

FIG. 8: HPLC-MS experiment of the cleavage of the beta ether bond of prodrug of formula VII, by purified LigF, giving 4-OHT. (A) negative control. (B) Time=0. (C) Time=1 hour. (D) Time=3 hours. (E) Time=44 hours

FIG. 9: Quantification of the conversion of the prodrug of formula VII (shown as isomers E and Z) to 4-OHT (shown both isomers Z and E) as measured by mass spectroscopy; relative abundance of molecules as percentage of total (y axis) at different time points (x axis).

FIG. 10: Co-culture assay of mouse intestinal tumor organoids expressing β-etherase with MEFs sensitive or not for 4-OHT (carrying a ubiquitous Cre-E^{RT2} protein or not) and treating with the compound of formula VII or 4-OHT treatment. Shown is the fraction of recombined cells, as measured by FACS analysis

FIG. 11: Normalized gene expression of TCF4-ER^{T2} fusion protein in cells administered with 4-OHT, the prodrug VII, at 1 and 10 μM, or a control with DMSO.

FIG. 12: Immunohistochemistry staining for EGFP of a section of mouse liver, expressing both Ub-Cre-ER^{T2} and the mTmG reporter cassette, treated with negative control (Oil, FIG. 12A), or with 1 μmol (FIG. 12B) or 5 μmol (FIG. 12C) of the compound of formula VII.

FIG. 13. Mouse liver metastasis formation assay as readout for TGFβ signaling inhibition and associated anti-cancer efficacy. C57BL/6 mice were implanted with MTOs and treatments with indicated compounds started two days after inoculation of tumor cells. The therapeutic efficacy of the indicated compounds was assessed by measuring their capacity to inhibit liver metastasis formation. Mice were treated with compound of formula I, compound of formula IV, compound 338 or galunisertib at the following molar equivalent doses; 9×, 3×, 1×, 0.3×. Control mice were treated with empty vehicle. The number of liver metastases (LiMs) was measured (y axis) after treatment. All mice were treated from day 2 to day 14 post inoculation of tumor cells and metastases count 4 weeks after MTO inoculation. Results were pooled from three independent experiments, denoted by the shape (square, triangle or circle) of the data points. P values against the pooled controls are indicated as: *: <0.05; **: <0.001; ***: <0.0001. Their values are: Gal 1×: 0.72; Gal 3×: 0.0107; Gal 9×: 0.0078; (IV) 1×: 0.075; (I) 0.3×: 0.026; (I) 1×: 0.0002; (I) 3×: 0.0004; (I) 9×: 0.035; 338 1×: 0.72; Mann-Whitney two-tailed.

FIG. 14: Therapeutic effects of the compounds in mice with overt metastatic disease. Immunocompetent mice were implanted with MTOs and mice were treated at day 16 post-inoculation when metastatic disease was overt. Mice were treated either with ALK5 inhibitors alone or in combination with antibodies against checkpoint molecule PD-1. The therapeutic efficacy of the indicated compounds was assessed by measuring their capacity to reduce the number of established liver metastases. The indicated compounds were given at equivalent 1× molar doses. For antibody administrations, mice were treated by intraperitoneal injection with 200 μg every 3 days. Control mice were treated with gavage vehicle and 200 μg IgG2a isotype control antibody. Mice were treated from day 16 to day 24. The number of liver metastases (LiMs, y-axis) was measured at the endpoint. Results were pooled from two independent experiments, denoted by the shape (square or circle) of the data points. P values against the pooled controls are indicated as: *: <0.05; **: <0.001; ***: <0.0001. Their values are: Gal 1×: 0.58; (I) 1×: 0.048; aPD-1: 0.0068; Gal+aPD-1: 0.18; (I)+aPD1: 0.0001; Mann-Whitney two-tailed.

FIG. 15: Therapeutic effects of the compounds in mice with overt metastatic disease. Immunocompetent mice were implanted with MTOs and mice were treated at day 16 post-inoculation when liver metastatic disease was overt. Therapeutic efficacy was assessed by measuring the percent of mice survival after treatment with the indicated compounds, which were given at equivalent 1× molar doses. Control mice were treated with gavage vehicle. All mice were treated from day 16 to day 24. Data come from the same experiments as shown in FIG. 14. P values against the pooled controls are: (I): 0.0018; Gal: 0.67; Log-rank test.

FIG. 16: Immunocompetent mice were implanted with MTOs and mice were treated at day 16 post-inoculation when metastatic disease was overt. The therapeutic efficacy of the indicated compounds was assessed by measuring the percent survival after treatment with anti-PD-1 antibodies (200 g every 3 days), or with a combination immunotherapy using anti-PD-1 antibodies plus compound of formula I or Galunisertib at 1× at equimolar dose. Control mice were treated with gavage vehicle and 200 μg IgG2a isotype control antibody. Mice were treated from day 16 to day 24. Data come from the same experiments as shown in FIG. 14. P values against the pooled controls are: (I)+aPD-1: <0.0001; aPD-1: 0.0048; Gal+aPD1: 0.43; Log-rank test.

FIG. 17: C57BL/6 mice were implanted with MTOs and treatments with indicated compounds started two days after inoculation of tumor cells. The therapeutic efficacy of the indicated compounds was assessed by measuring their capacity to inhibit liver metastasis formation. Mice were treated with compound of formula I, compound 338 or compound 337 at 1×. Control mice were treated with empty vehicle. The number of liver metastases (LiMs) was measured (y axis) after treatment. All mice were treated from day 2 to day 18 post-inoculation of tumor cells. Number of liver metastases were counted at 21 days after MTO inoculation. Ns is not significant, * indicates <0.05 by Mann-Whitney two-tailed test.

### DESCRIPTION OF THE INVENTION

Present invention refers to a compound of formula (I), or a pharmaceutically acceptable salt, or a pharmaceutically acceptable solvate or a polymorph or a cocrystal thereof:

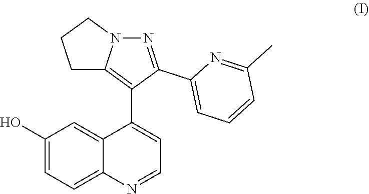 [SMILES: Cc1cccc(-c2nn3c(c2-c2ccnc4ccc(O)cc24)CCC3)n1]

In particular, present invention refers to a compound of formula I, or a pharmaceutically acceptable salt, or a pharmaceutically acceptable solvate thereof.

Present invention also relates to a pharmaceutical composition comprising an effective amount of a compound of formula I, or a pharmaceutically acceptable salt, or a pharmaceutically acceptable solvate or a polymorph or a cocrystal thereof, and at least one pharmaceutical acceptable excipient.

Present invention refers to a compound of formula I, or a pharmaceutical composition comprising said compound, for use as a medicament, in particular for use in the treatment of a disease responsive to the inhibition of the TGFβ pathway, or to the inhibition of TGFβ signaling. For the purposes of present invention, the term “inhibitor of the TGFβ pathway” is equivalent to the term “inhibitor of the TGFβ signaling pathway” and to the term “TGFβ inhibitor”, and the three terms are used indistinctively throughout the description. To this end, for the purpose of present invention, the term “disease responsive to the inhibition of the TGFβ pathway” is, thus, equivalent to the term “disease responsive to the inhibition of the TGFβ signaling pathway”.

The compound of formula I is an inhibitor of the TGFβ pathway. Inhibitors of the TGFβ signaling pathway may inhibit one or more TGFβ signaling pathway receptors selected from the group consisting of ALK1, ALK2, ALK3, ALK4, ALK5, ALK6, ALK7, TGFBR2, BMPR2, ACVR2A, and ACVR2B. Accordingly, present invention also refers to a compound of formula I for use in the treatment of a disease responsive to inhibition of one or more TGFβ signaling pathway receptors, wherein the TGFβ signaling pathway receptors are TGFβ signaling pathway type I receptors or TGFβ signaling pathway type II receptors and are selected from the group consisting of ALK1, ALK2, ALK3, ALK4, ALK5, ALK6, ALK7, TGFBR2, BMPR2, ACVR2A, and ACVR2B. Therefore, present invention refers to a compound of formula I for use as an inhibitor of one or more TGFβ signaling pathway receptors selected from the group consisting of ALK1, ALK2, ALK3, ALK4, ALK5, ALK6, ALK7, TGFBR2, BMPR2, ACVR2A, and ACVR2B.

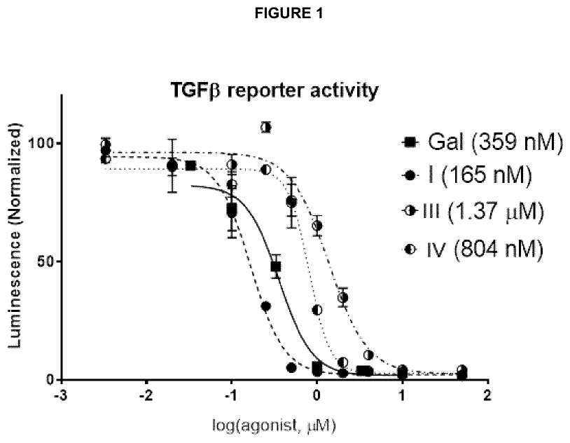

Results included in Table 1 of Example 2, show that the compound of formula I has a surprisingly higher (8.6 times) binding affinity for ALK5 than Galunisertib. This comparison has been made taking into account published data (Jonathan M et al., Oncotarget, 2018, 9 (6): 6659-6677) using the same technology (KINOMEscan™). Also, the ALK5 kinase enzymatic activity inhibition as well as the TGFβ signaling inhibitory capacity in cultured cells of the compound of formula I is about 2.5 times that of Galunisertib, as seen in Table 2 of example 2, and FIG. 1. Furthermore, selectivity assays (KINOMEscan™ technology) show that the compound of formula I feature a selectivity for ALK5 greater than 300-fold against ALK1, ALK2 and ALK3, 100-fold against TGFBR2 and 5 and 8-fold against ACVR2B and ALK6 respectively, as described in example 2 (Table 1). Moreover, the compound of formula I was more selective than Galunisertib for all proteins tested.

Accordingly, the structural differences of the compound of formula I with compounds of the prior art, surprisingly result in a strongly improved TGFβ signaling inhibitory capacity, associated with a better inhibition of ALK5. The compound of formula I is also more selective, comparing binding affinity to that of ALK5, than the prior art compound. On the other hand, the compound of formula I has cleared toxicity screens, including screens for genotoxicity, perturbation of proteins strongly linked to adverse drug reactions (SafetyScreen44™, as described in Example 2), and in vivo (mouse) toxicity (Example 4) showing that the compound of formula I provides an improved toxicity profile compared to Galunisertib, potentially avoiding the need for intermittent treatment.

The compound of formula I may be synthetized from a p-aniline with a masked or protected phenol in para-position. In this sense, the synthesis of the compound of formula I may use different routes as seen in the retrosynthetic analysis of scheme 1:

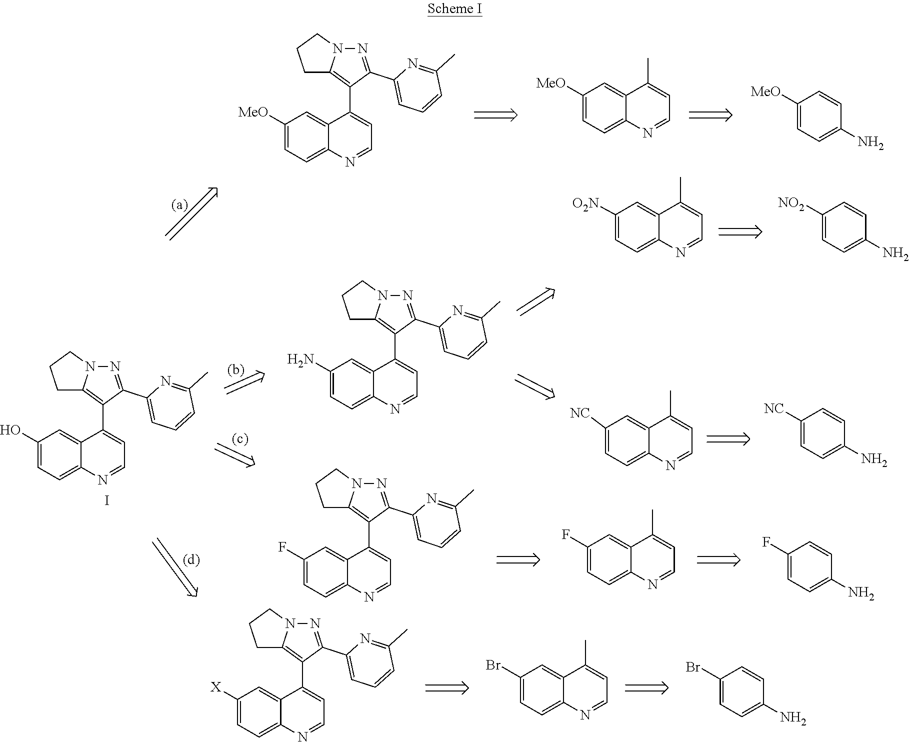

Example 1 includes all the synthetic details corresponding to the preparation of the compound of formula I according to the preferred synthetic routes (a) and (d), but the synthetic routes (b) and (c) are also suitable routes to obtain the compound of formula I, according to present invention.

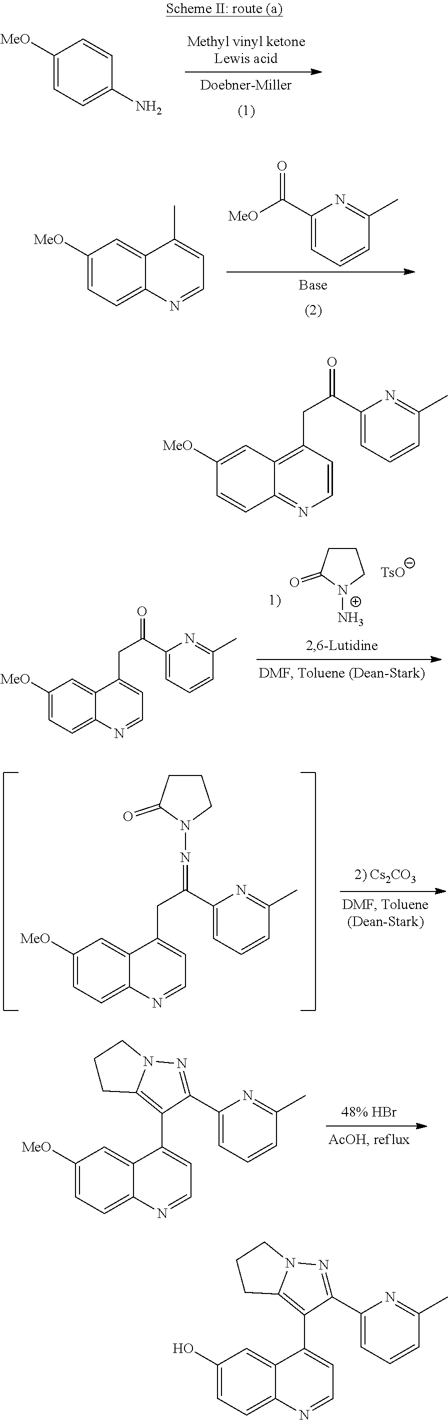

Route (a) comprises the synthesis of the compound of formula I, departing from a protected para-hydroxy aniline, wherein said protected phenol is only deprotected at the end. In a first step 6-methoxy-4-methylquinoline is obtained via the Doebner-Miller reaction in the presence of a Lewis acid catalyst. The Doebner-Miller reaction is well known in the art (Bergström, F. W. Chem. Rev. 1944, 35, 153).

In a second step the acylation of 6-methoxy-4-methylquinoline is carried out adding methyl 6-methylpicolinate in the presence of a strong base (pKa of conjugate acid higher than 17) and suitable conditions.

Finally, the transformation into the methyl-protected compound of formula I, of the ketone intermediate is carried out, first using 1-aminopyrrolidin-2-one and a base such as lutidine, under Dean-Stark conditions to obtain the hydrazone intermediate, and subsequently using suitable conditions for cyclisation which upon dehydration under Dean-Stark conditions results in the methyl-protected compound of formula I.

The methyl group may be removed by refluxing in HBr in AcOH, although other usual deprotection conditions such as BBr₃, BCl₃, TMSCl/NaI or BF₃/RSH can be used.

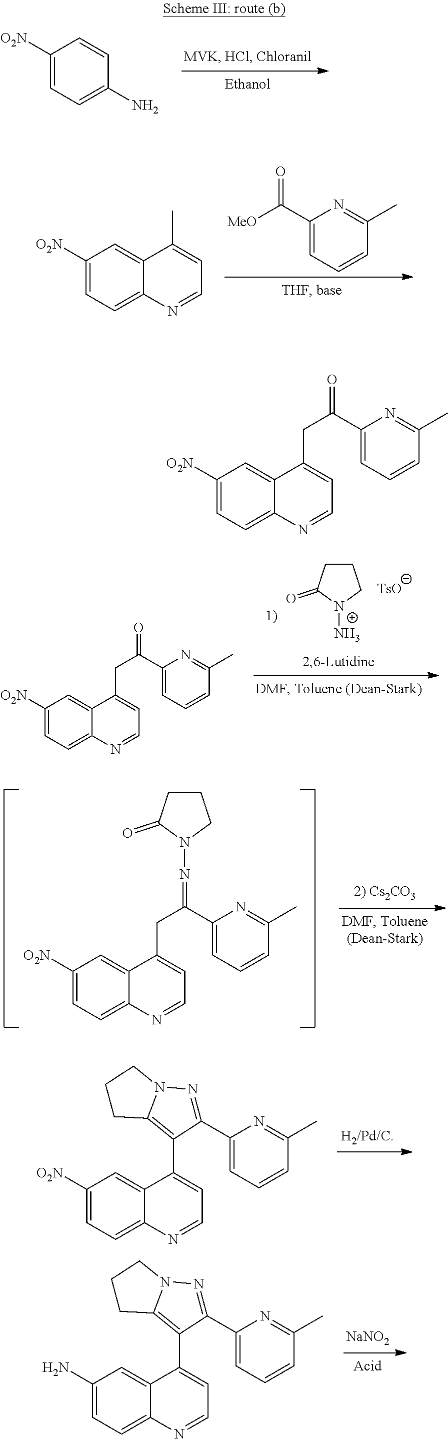

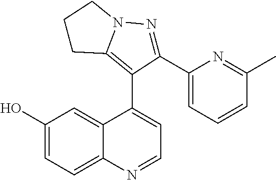 [SMILES: Cc1cccc(-c2nn3c(c2-c2ccnc4ccc(O)cc24)CCC3)n1]

Route (b) comprises an equivalent first step to route (a), where a modified Doebner-Miller reaction of para-nitroaniline is carried out. The second step also includes the acylation of 6-nitro-4-methylquinoline adding methyl 6-methylpicolinate in the presence of a base. The subsequent step to perform the formation of the hydrazone and cyclization was carried out in an equivalent manner to that of route (a). Reduction of the nitro-derivative is carried out by any of the known methods (H₂/Pd/C; Sn/HCl, etc.) and conversion of the amino group into a phenol by hydrolysis of a diazonium salt.

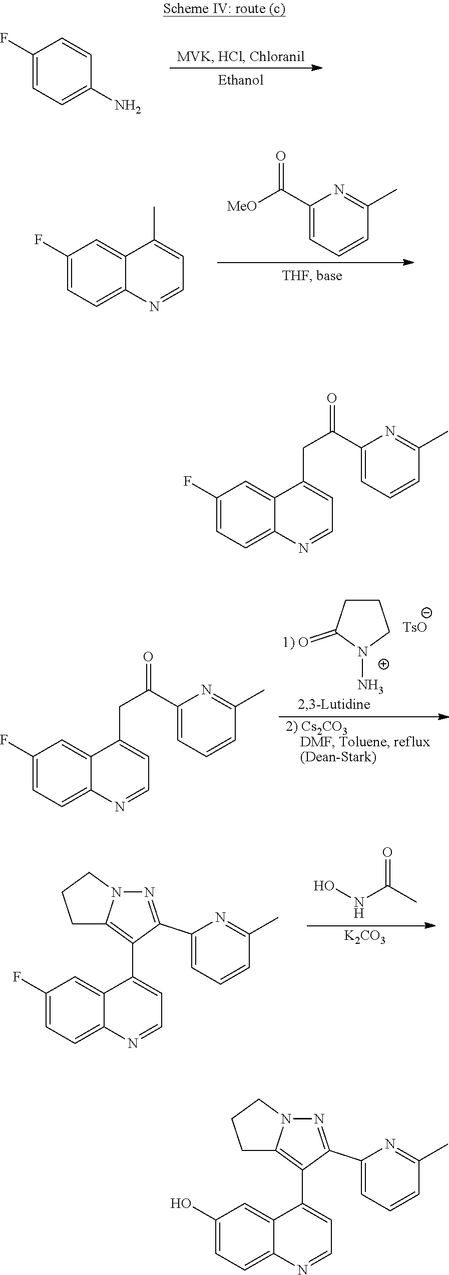

Route (c) comprises an equivalent first step to route (a), where the modified Doebner-Miller reaction of is carried out using a para-fluoroaniline and an oxidant such as chloranil. The second step also includes the acylation of 6-fluoro-4-methylquinoline that was carried out adding methyl 6-methylpicolinate in the presence of a strong base. The subsequent step to perform the formation of the hydrazone and cyclization is carried out in an equivalent manner to that of route (a). Conversion of the fluoroderivative into a phenol can be done in various ways including transition metal catalysis or the use of acetoxyhydroxamic acid as described by Fier and Maloney (Org. Lett. 2016, 18, 2244).

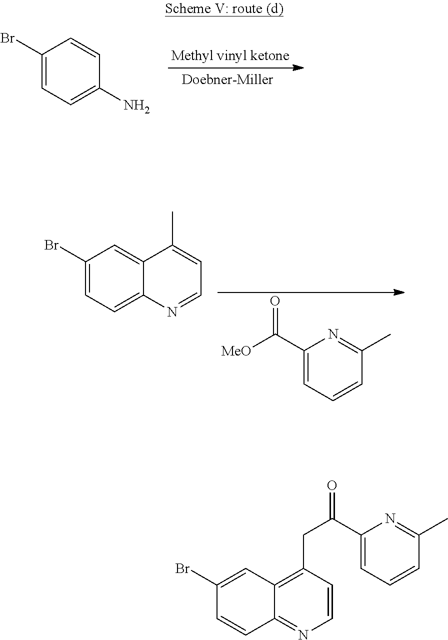 [SMILES: Nc1ccc(Br)cc1 ; Cc1ccnc2ccc(Br)cc12 ; COC(=O)c1cccc(C)n1 ; Cc1cccc(C(=O)Cc2ccnc3ccc(Br)cc23)n1]

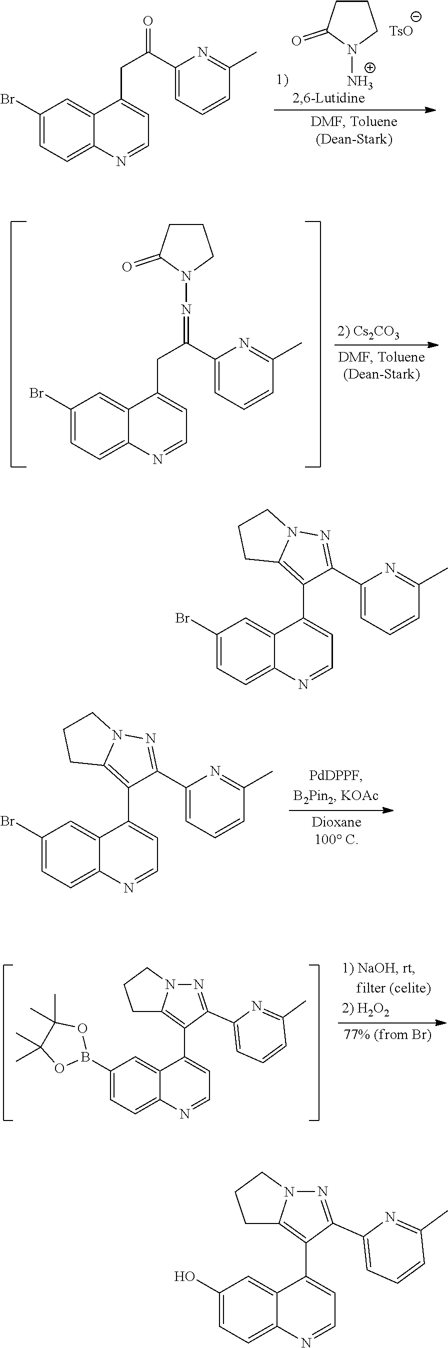

Route (d) comprises an equivalent first step to route (a), where a modified Doebner-Miller reaction of para-bromoaniline is carried out using air as oxidant. The second step also includes the acylation of 6-bromo-4-methylquinoline carried out by adding methyl 6-methylpicolinate in the presence of a strong base. The subsequent step to perform the formation of the hydrazone and cyclization is carried out in an equivalent manner to that of route (a). Subsequently, the compound of formula I can be obtained using a Miyaura borylation (T. Ishiyama, M. Murata, N. Miyaura, J. Org. Chem. 1995, 60:7508-7510) wherein the aryl bromide is converted to the corresponding boronic esters via Pd catalyzed cross coupling reactions with diboronyl esters such as Bis(pinacolato)diboron (B₂Pin₂). The coupling proceeds via Pd(0)/Pd(II) mechanism, therefore any source of Pd(0) can perform the reaction. In a preferred embodiment, [1,1′-bis(diphenyl phosphino)ferrocene]dichloropalladium (Pd(DPPF)Cl₂) is the appropriate reagent.

Without any further purification, the boronic ester may be oxidised with hydrogen peroxide, and after subsequent hydrolysis in the basic media, the compound of formula I is obtained.

Present invention also refers to prodrugs. Prodrugs are compounds deriving from active medicaments or drugs (parent compound) which have been modified to modulate properties such as solubility, absorption, distribution, metabolism, excretion and toxicity. Therefore, prodrugs are compounds which include the parent compound attached to a fragment which reduces or blocks the activity, whilst at the same time modulate one or more properties such as solubility, absorption, distribution, metabolism, excretion and toxicity.

In particular, present invention refers to prodrugs of the compound of formula I.

An embodiment of present invention refers to a prodrug of the compound of formula I wherein said prodrug has formula II, or a pharmaceutically acceptable salt, or a pharmaceutically acceptable solvate or a polymorph or a cocrystal thereof:

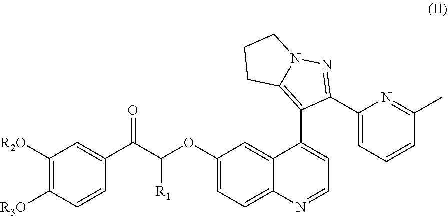

wherein
R₁ is selected from the group consisting of H, alkyl, cycloalkyl, haloalkyl, hydroxyalkyl, aminoalkyl, alkanoylamino, alkoxy, aryl, alkylaryl, arylalkyl, amino, N-alkylamino and N,N-dialkylamino; and
R₂ and R₃ are each independently selected from the group consisting of H, alkyl and haloalkyl.

One embodiment refers to a prodrug of the compound of formula I, wherein said prodrug has formula II, or a pharmaceutically acceptable salt, or a pharmaceutically acceptable solvate thereof, wherein R₁ is selected from the group consisting of H, alkyl, cycloalkyl, haloalkyl, hydroxyalkyl, aminoalkyl, alkanoylamino, alkoxy, aryl, alkylaryl, arylalkyl, amino, N-alkylamino and N,N-dialkylamino; R₂ is methyl and R₃ is H. In another embodiment R₁ is selected from the group consisting of H, alkyl, haloalkyl, aminoalkyl and hydroxyalkyl; R₂ is methyl and R₃ is H.

In a preferred embodiment of present invention, R₁ is H, R₂ is methyl and R₃ is H, and the prodrug of the compound of formula I is a prodrug of formula (III) or a pharmaceutically acceptable salt, or a pharmaceutically acceptable solvate or a polymorph or a cocrystal thereof:

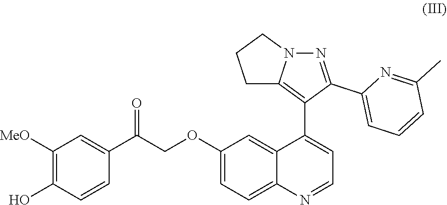 [SMILES: COc1cc(C(=O)COc2ccc3nccc(-c4c(-c5cccc(C)n5)nn5c4CCC5)c3c2)ccc1O]

Preferably, the prodrug of the compound of formula I is a prodrug of formula (III) or a pharmaceutically acceptable salt, or a pharmaceutically acceptable solvate thereof. The prodrug of formula III is obtained, as illustrated in example 1, by alkylation reaction of the phenol group of the compound of formula I with a halogenated derivative of acetovanillone having the acetovanillone phenol group protected, and subsequent deprotection of the acetovanillone moiety.

In another preferred embodiment the prodrug of the compound of formula I is a prodrug of formula (IV) or a pharmaceutically acceptable salt, or a pharmaceutically acceptable solvate or a polymorph or a cocrystal thereof:

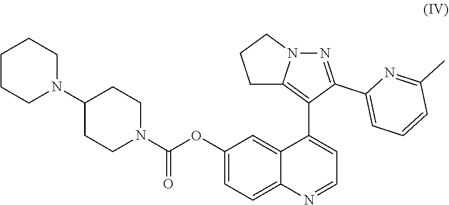 [SMILES: Cc1cccc(-c2nn3c(c2-c2ccnc4ccc(OC(=O)N5CCC(N6CCCCC6)CC5)cc24)CCC3)n1]

Preferably, the prodrug of the compound of formula I is a prodrug of formula (IV) or a pharmaceutically acceptable salt, or a pharmaceutically acceptable solvate thereof. The prodrug of formula IV is obtained by reaction of the compound of formula I with the 4-piperidinopiperidine-1-carbonyl chloride as illustrated in example 1.

The term “comprises” is to be understood as to include includes a group of features (A, B, C), however it does not exclude embodiments in which other features are also included, as long as they do not render said embodiments unworkable. Additionally, for the purposes of present invention, said term “comprises” also encompasses embodiments in which only said features (A, B, C) are included, in which case may be replaced by the term “consisting of”.

The term “pharmaceutically acceptable salt” refers to any pharmaceutically acceptable salt, which upon administration to the patient is capable of providing (directly or indirectly) a compound as described herein.

Such salts preferably are acid addition salts or basic addition salts. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulphonate and p-toluenesulphonate. Examples of the basic addition salts include inorganic salts such as, for example, sodium, potassium, calcium and ammonium salts, and organic basic salts such as, for example, ethylenediamine, ethanolamine, N,N-dialkylenethanolamine, triethanolamine and basic aminoacids salts. However, it will be appreciated that non-pharmaceutically acceptable salts also fall within the scope of the invention since those may be useful in the preparation of pharmaceutically acceptable salts. Procedures for salt formation are conventional in the art.

In an embodiment of present invention, the pharmaceutically acceptable salt of the compound of formula I is a basic addition salt or an acid addition salt. In a preferred embodiment said pharmaceutically acceptable salt of the compound of formula I is selected from the group consisting of sodium, potassium, calcium, ammonium, alkyl ammonium, dialkyl ammonium, trialkyl ammonium, ethanolamine, N,N-dialkylenethanolamine and amino acid salt.

In another preferred embodiment said pharmaceutically acceptable salt of the compound of formula I is selected from the group consisting of hydrochloride, hydrobromide, hydroiodide, sulphate, bisulphate, nitrate, phosphate, hydrogen phosphate, acetate, trifluoroacetate, maleate, fumarate, citrate, lactate, oxalate, succinate, gluconate, tartrate, maleate, mandelate, methanesulphonate and p-toluenesulphonate.

In an embodiment of present invention, the pharmaceutically acceptable salt of the prodrug of formula II, and in particular of the prodrug of formula Ill, is an acid addition salt. In a preferred embodiment said pharmaceutically acceptable salt of the compound of formula II, and in particular of the prodrug of formula Ill, is selected from the group consisting of hydrochloride, hydrobromide, hydroiodide, sulphate, bisulphate, nitrate, phosphate, hydrogen phosphate, acetate, trifluoroacetate, maleate, fumarate, citrate, lactate, oxalate, succinate, gluconate, tartrate, maleate, mandelate, methanesulphonate and p-toluenesulphonate.

In an embodiment of present invention, the pharmaceutically acceptable salt of the compound of formula IV is a basic addition salt or an acid addition salt. In a preferred embodiment said pharmaceutically acceptable salt of the compound of formula IV is selected from the group consisting of hydrochloride, hydrobromide, hydroiodide, sulphate, phosphate, acetate, trifluoroacetate, maleate, fumarate, citrate, oxalate, succinate, tartrate, malate, mandelate, methanesulphonate and p-toluenesulphonate. Most preferably the pharmaceutically acceptable salt of the compound of formula IV is a hydrochloride salt.

In another preferred embodiment said pharmaceutically acceptable salt of the compound of formula IV is selected from the group consisting of sodium, potassium, calcium, ammonium, alkyl ammonium, dialkyl ammonium, trialkyl ammonium, ethanolamine, N,N-dialkylenethanolamine and amino acid salt.

The term “solvate” in accordance with this invention should be understood as meaning any form of the active compound in accordance with the invention in which said compound is bonded by a non-covalent bond to another molecule (normally a polar solvent), including especially hydrates and alcoholates. In an embodiment of present invention, the pharmaceutically acceptable solvate is an alcoholate. In an embodiment of present invention, the pharmaceutically acceptable solvate is a hydrate. In an embodiment of present invention, the compound of formula I, the prodrug of formula II, the prodrug of formula III or the prodrug of formula IV are monohydrates.

The term “polymorph” refers to crystalline forms having the same chemical composition but different spatial arrangements of the molecules, atoms, and/or ions forming the crystal. Accordingly, the same compound of formula I, or a pharmaceutical salt, or a pharmaceutical solvate thereof, may include different crystalline polymorphs depending on spatial arrangement of the atoms or ions forming said crystalline form.

The compound of formula I is an inhibitor of the TGFβ signaling pathway with improved inhibitory capacity and low toxicity characteristics, as shown in examples 2 to 4 and examples 8 to 10.

Accordingly, one embodiment refers to a compound of formula I, or a pharmaceutically acceptable salt, or a pharmaceutically acceptable solvate or a polymorph or a cocrystal thereof, for use in the treatment of diseases responsive to TGFβ signaling pathway inhibition. Preferably to a compound of formula I, or a pharmaceutically acceptable salt, or a pharmaceutically acceptable solvate thereof, for use in the treatment of diseases responsive to TGFβ pathway inhibition.

Prodrugs are transformed into the parent compound following cleavage of a fragment attached to said medicament or drug. This can be done by a variety of enzymes.

The prodrugs of the compound of formula I when metabolized, release the compound of formula I, and accordingly are administered locally to the tumor, preventing in this manner the systemic pathway inhibition which, results in adverse effects when continuous inhibition of the TGFβ signaling pathway is conducted. Accordingly, a prodrug of a compound of formula I is useful also in the prevention and/or treatment of a disease responsive to the inhibition of the TGFβ pathway.

As seen in FIG. 1 and table 2 the prodrugs of the compound of formula I, the prodrugs of formula II, formula Ill, and formula IV, have a reduced TGFβ signaling inhibiting capacity in vitro, but deliver the compound of formula I in vivo, i.e. the prodrugs of formula II, formula Ill, and formula IV, are prodrugs which are metabolized in vivo, releasing the active compound of formula I.

In fact, the 4-piperidinopiperidine group of the prodrug of formula IV provides water solubility and is cleaved via hydrolysis by carboxylesterases in vivo expressed in e.g. liver and intestinal cells.

Accordingly, when the prodrug of formula IV, is administered, the compound of formula I is released upon hydrolysis by carboxylesterases in liver and intestinal cells. The main advantage is that it allows the administration of high local doses and TGFβ signaling inhibition, with reduced systemic toxicity.

On the other hand, the acetovanillone-derived fragment of the prodrugs of formula II, and in particular of the prodrug of formula Ill, can be oxidized and cleaved by enzymes, such as the cytochrome P450 enzyme family (mainly present in the liver), which hydrolyze the ether bond delivering the compound of formula I. On the other hand, the compound of formula I can be also obtained from the prodrug of formula II thereof, using specific beta-etherases from non-mammalian sources, such as bacterium Sphingobium sp. strain SYK-6 enzymes LigE and LigF, or fungus Dichomitus squalens Ds-GST1, which can be administered in conjunction with the prodrug of formula II, and preferably with the prodrug of formula Ill.

Accordingly, an embodiment refers to the prodrug of formula II for use in the treatment of a disease responsive to the inhibition of the TGFβ pathway, wherein a beta-etherase enzyme is administered together, subsequently or prior to the compound of formula II. A preferred embodiment refers to a prodrug of formula III for use in the treatment of a disease responsive to the inhibition of the TGFβ pathway, wherein a beta-etherase enzyme is administered together or subsequently to the compound of formula Ill. Example 6 shows how LigF, a beta-etherase enzyme is capable of hydrolyzing the ether bond cleaving the acetovanillone fragment in a model experiment carried out with the acetovanillone ether of 4-methylumbelliferone and with the acetovanillone ether of 4-hydroxytamoxifen.

In a particular embodiment the beta-etherase enzyme is administered in cells expressing said beta-etherase enzyme, as cell therapy, for instance, adoptive T-cell transfer or dendritic cell vaccination. Alternatively, in another particular embodiment the beta-etherase enzyme is administered coupled to an antibody which is tumor or stroma specific, optionally with glutathione. Certain tissues such as the lung lining comprise high extracellular levels of gluthatione, and accordingly the administration of gluthatione is not required.

The prodrugs of formula II, formula Ill, and formula IV, or pharmaceutically acceptable salts or solvates thereof, have a strongly reduced activity as TGFβ signaling pathway inhibitors so that they can be administered with little or no effect on the TGFβ pathway, unless the beta ether bond is cleaved.

When the prodrugs of formula II, or formula III, or pharmaceutically acceptable salts or solvates thereof, are administered, the compound of formula I is released upon localized administration of a beta-etherase, in the target tumor stroma. The main advantage is that it allows the administration of high local doses and TGFβ signaling pathway inhibition, with reduced systemic toxicity.

On the other hand, the prodrug of the compound of formula I, having formula IV, or a pharmaceutically acceptable salt thereof, also provides reduced TGFβ signaling inhibitory activity in vitro and, when administered in vivo, is hydrolyzed and give rise to the compound of formula I in vivo by enzymatic action of carboxylesterases expressed in different body tissue cells such as liver and intestinal cells.

The compound of formula I was then found to be effective at a much lower dose in in vivo experiments (10-fold lower, in fact) than the effective Galunisertib dose. Moreover, at effective doses, the compound of formula I had no toxicity (gross in vivo or pathological on tissue sections), whereas Galunisertib at murine-relevant doses did have toxicities (see examples 2 to 4). As seen in FIG. 1 and Table 2 prodrugs of compound of formula I, such as the prodrug of formula III and the prodrug of formula IV, have up to 10 times lower TGFβ signaling inhibitory capacity in vitro, which demonstrate that the TGFβ signaling inhibitory capacity of the compound of formula I is successfully caged or blocked.

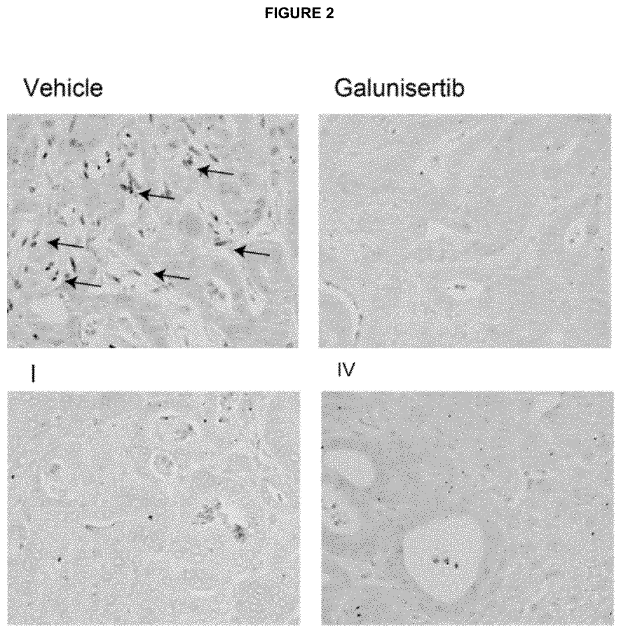

FIG. 2 shows the high TGFβ signaling inhibitory activity of the compound of formula I, whereas, the prodrug of formula IV, which did not show significant TGFβ signaling inhibitory activity in vitro, provides a high TGFβ signaling pathway inhibitory activity in vivo, due in vivo hydrolysis of said prodrug delivering the active compound of formula I.

Accordingly, one embodiment of present invention refers to a prodrug of the compound of formula I, wherein said prodrug is a prodrug of formula II, or a pharmaceutically acceptable salt, or a pharmaceutically acceptable solvate or a polymorph or a cocrystal thereof, for use as a medicament, in particular for use in the treatment of a disease responsive to TGFβ signaling. Preferably said prodrug is a prodrug of formula Ill. A preferred embodiment refers to a compound of formula Ill, or a pharmaceutically acceptable salt, or a pharmaceutically acceptable solvate thereof, for use as a medicament, in particular for use in the treatment of a disease responsive to TGFβ signaling pathway inhibition.

Another embodiment refers to the use of a compound of formula I, or a pharmaceutically acceptable salt, or a pharmaceutically acceptable solvate, or a polymorph or a cocrystal thereof, or a composition comprising said compound of formula I, in the manufacture of a medicament for the treatment of a disease responsive to TGFβ signaling pathway inhibition.

An additional embodiment refers to a pharmaceutical composition comprising a compound of formula I, or a pharmaceutically acceptable salt, or a pharmaceutically acceptable solvate, or a polymorph or a cocrystal thereof, and at least one pharmaceutically acceptable excipient, and optionally one additional active ingredient or therapeutic agent.

For the purposes of present invention, the terms “active ingredient” or “therapeutic agent” refer to another compound or therapy which improves or enhances the therapeutic effect of the compound of the invention, or of the prodrugs thereof.

Another embodiment of present invention refers to a prodrug of the compound of formula I, wherein said prodrug is a prodrug of formula IV, or a pharmaceutically acceptable salt, or a pharmaceutically acceptable solvate or a polymorph or a cocrystal thereof, for use as a medicament, in particular for use in the treatment of a disease responsive to TGFβ signaling pathway inhibition. Preferably, a compound of formula IV, or a pharmaceutically acceptable salt, or a pharmaceutically acceptable solvate thereof, for use as a medicament, in particular for use in the treatment of a disease responsive to TGFβ signaling pathway inhibition.

An alternative embodiment of the present invention refers to the use, as medicaments, of said compound of formula I or prodrugs thereof, prodrug of formula II, formula III and formula IV, or pharmaceutically acceptable salts or solvates thereof, alone or formulated in compositions, particularly pharmaceutical compositions, that comprise at least said compound of formula I or prodrugs thereof, prodrug of formula II, formula III and formula IV, combined with at least one other active compound or therapeutic agent having additive or synergistic biological activities.

In this sense, one embodiment of present invention refers to the compound of formula I, or prodrugs thereof, prodrug of formula II, formula III and formula IV, or pharmaceutically acceptable salts or solvates thereof, or to a pharmaceutical composition comprising at least one of said compounds, for use in the prevention and/or treatment of a disease responsive to TGFβ signaling pathway inhibition, in combination with other cancer targeted therapies, or with chemotherapy.

For the purpose of present invention, the term “chemotherapy” refers to any treatment which acts stopping the growth of cancer cells, either by killing the cells or by stopping them from dividing.

Thus, one embodiment of present invention refers to the compound of formula I, or prodrugs thereof, prodrug of formula II, formula III and formula IV, or pharmaceutically acceptable salts or solvates thereof, or to a pharmaceutical composition comprising at least one of said compounds, for use in combination with chemotherapy, or with a chemotherapeutic agent. In particular, in combination with one chemotherapeutic agent selected from the group consisting of platinum-based antineoplastic agents, anti-mitotic chemotherapeutic agents, a poly adenosine diphosphate ribose polymerase (PARP) inhibitor, type I topoisomerase inhibitors, type II topoisomerase inhibitors, epothilones, cycloskeletal disruptors, alkylating agents, epothilones, histone deacetylase inhibitors, kinase inhibitors, antifolates, kinase inhibitors, peptide antibiotics, retinoids, vinca alkaloids and thymidylate synthase inhibitors. Preferably, the chemotherapeutic agent is selected from the group consisting of cyclophosphamide, ifosfamide, busulfan, temozolomide, mechlorethamine, chlorambucil, melphalan, dacarbazine, daunorubicin, doxorubicin, daunorubicin, epirubicin, idarubicin, mitoxantrone, valrubicin, paclitaxel, docetaxel, abraxane, taxotere, epothilone, vorinostat, romidepsin, irinotecan, topotecan, camptothecin, exatecan, lurtotecan, etoposide, teniposide, tafluposide, bortezomib, erlotinib, gefitinib, imatinib, vemurafenib, vismodegib, azacitadine, azathioprine, capecitabine, cytarabine, cladribine, fludarabine, doxifluridine, fluorouracil, gemcitabine, hydroxyurea, mercaptopurine, methotrexate, pemetrexed, tioguanine, bleomycin, actinomycin, carboplatin, cisplatin, oxaliplatin, tretinoin, alitretinoin, bexarotene, vinblastine, vincristine, vindesine and vinorelbine.

For purpose of present invention, the term “cancer targeted therapies” refers to any type of therapy that targets the cancer's specific genes, proteins, or the tissue environment that contributes to cancer growth and survival. One embodiment of the invention refers, therefore, to a compound of formula I, or prodrugs thereof, prodrug of formula II, formula III and formula IV, or pharmaceutically acceptable salts or solvates thereof, or a composition comprising at least one of said compounds, for use in the prevention and/or treatment of a disease responsive to TGFβ signaling pathway inhibition in combination cancer targeted therapy, preferably, said cancer targeted therapy is selected from the group consisting of hormone therapies, signal transduction inhibitors, gene expression modulators, apoptosis inducers, angiogenesis inhibitors, immunotherapeutic agents, monoclonal antibodies delivering toxic molecules, cancer therapy and gene therapy.

One embodiment of the invention refers, therefore, to a compound of formula I, or prodrugs thereof, prodrug of formula II, formula III and formula IV, or pharmaceutically acceptable salts or solvates thereof, or a composition comprising at least one of said compounds, for use in the prevention and/or treatment of a disease responsive to TGFβ signaling pathway inhibition in combination with immunotherapy.

For the purposes of present invention, the term “immunotherapy” refers to immunotherapeutic agents, compounds or ingredients which activate the immune system to kill cancer cells. More preferably the immunotherapy or immunotherapeutic agent is an immune checkpoint inhibitors and even more preferably an anti PD-1 or PDL1 inhibitor.

In this sense, another embodiment of the invention refers to a compound of formula I, or prodrugs thereof, prodrug of formula II, formula III and formula IV, or pharmaceutically acceptable salts or solvates thereof, or a pharmaceutical composition comprising at least one of said compounds, for use in the prevention and/or treatment of a disease responsive to TGFβ signaling pathway inhibition in combination with an immunotherapy agent, preferably an immune checkpoint inhibitor; more preferably, an anti-PD1 or PDL1 inhibitor; yet more preferably, an anti-PD1 antibody.

The programmed cell death protein 1 (PD-1) is a transmembrane protein expressed on T cells, which functions as an immune checkpoint that negatively regulates T-cell activation and causes down regulation of the immune system. Upon administration, anti-PD-1 monoclonal antibodies bind to and inhibit PD-1 and its downstream signaling pathways, restoring the immune function through the activation of T-cells and cell-mediated immune responses against tumor cells.

In a preferred embodiment said at least one other active compound or therapeutic agent is selected from the group consisting of an oncolytic virus inhibiting PD1, and antibody anti PD-1 or PDL1, a Bispecific Monoclonal Antibody inhibiting PD-1 or PDL1, a small molecule inhibiting PD-1 or PDL-1, a protein antagonizing PD-1 and a Vaccine targeting PD-1.

In a preferred embodiment said at least one other active compound or therapeutic agent is selected from the group consisting of nivolumab, pembrolizumab, cemiplimab, camrelizumab, sintilimab, toripalimab, tislelizumab, atezolimumab, avelumab, durvalumab, KN-035, CK-301, AUNP12, CA-170, BMS-986189, MDX-1105, MED10680, LY3300054, LY-3434172, APL-502, bintrafusp alfa, CS-1001, SHR-1316, BGBA-333, CX-072, GEN-1046, GS-4224, 10-103, KD-005, KLA-167, KN-046, lazertinib, STIA-1014, WP-1066, ADG-104, BCD-135 BCD-135, FAZ-053, FPT-155, FS-118, HLX-20, IBI-318, INBRX-105, INCB-86550, JS-003, Iodapolimab, LP-002, MCLA-145, MSB-2311, RG-6084, SHR-1701, SL-279252, STIA-1015, LYN-00102, ACE-05, AP-201, AP-505, AVA-004, AVA-021, BBI-801, BH-2996h, BH-29XX, CA-327, CDX-527, CTX-8371, DPDL-1E, DSP-105, DSP-106, Gensci-047, HS-636, IBI-322, IBI-323, IKT-201, IMM-2505, JBI-426, KD-033, KY-1043, ND-021, PH-790, PMC-122, PRS-344, SNA-02, STT-01, TJL-14B, TJL-1C4, TJL-1D5, TJL-1H3, TJL-117, Toca-521, VXM-10, Y-111, ABM-101, ABP-160, AVA-027, AVA-040, DB-002, DB-003, IMC-2101, IMC-2102, IMM-2510, TE-7212, TXB-4BC3, YBL-007, YBL-008, ALN-PDL, BH-2941, BMS-936559 CBA-0710, GXP-2, HTI-1316, IGEM-P, IMM-25, IMM-2502, IMM-2503, IMM-2504, 10-104, KD-036, KY-1003, KY-1055, MSB-002, PMC-305, STIA-100X, STIA-1010, STIA-1011, STIA-1012, STIB-010X, LY-3415244, AK-103, AK-104, AK-105, AK-106, dostarlimab, HLX-10, prolgolimab, spartalizumab, APL-501, balstilimab, BAT-1306, BI-754091, cetrelimab, GLS-010, MGA-012, pidilizumab, SCTI-10A, AMG-404, BCD-217, budigalimab, CC-90006, CS-1003, F-520, HAB-21, HX-009, IBI-318, JTX-4014, LY-3434172, LZM-009, MEDI-5752, MGD-013, MGD-019, ONO-4685, RO-7121661, sasanlimab, sulituzumab, Sym-021, XmAb-20717, XmAb-23104, AK-112, PSB-205, AK-123, ALPN-202, AM-0001, ANB-030, BH-2922, BH-2950, BH-2954, BH-2996h, CB-201, CB-213, CBT-103, CBT-107, CTX-8371, GNR-051, HEISCOIII-003, IBI-319, KEY-Vaxx, LD-01, LD-10, MD-402, mDX-400, OT-2, PC-101, PEGMP-7, PH-762, PRS-332, PT-001, RO-7247669, SG-001, TSR-075, AT-16201, DB-004, IKT-202, IMT-200, KF-082, LBL-006, SSI-361, YBL-006, JY-034, TRS-007, AMP-224, AUR-012, BGB-108, BH-2941, BLSM-101, CX-188, ENUM-244C8, ENUM-388D4, IMM-1802, MEDI-0680, SNA-01, STIA-1110 and Sym-016.

Alternatively said compositions can be formulated with at least one inert ingredient as a carrier or excipient such as: cosolvents, surfactants, oils, humectants, emollients, preservatives, stabilizers and antioxidants. Any pharmacologically acceptable buffer may be used, e.g. TRIS or phosphate buffers.

A further embodiment refers to a pharmaceutical composition comprising a prodrug of the compound of formula I, said prodrug having formula II, or formula Ill, or a pharmaceutically acceptable salt, or a pharmaceutically acceptable solvate, or a polymorph or a cocrystal thereof, and at least one pharmaceutically acceptable excipient, and optionally one additional active ingredient. Another further embodiment refers to a pharmaceutical composition comprising a prodrug of the compound of formula I, said prodrug having formula IV, or a pharmaceutically acceptable salt, or a pharmaceutically acceptable solvate, or a polymorph or a cocrystal thereof, and at least one pharmaceutically acceptable excipient, and optionally one additional active ingredient.

Another embodiment refers to the use of a prodrug of the compound of formula I, or a pharmaceutically acceptable salt, or a pharmaceutically acceptable solvate, or a polymorph or a cocrystal thereof, or a composition comprising said prodrug of the compound of formula I, in the manufacture of a medicament for the treatment of a disease responsive to TGFβ signaling inhibition. A preferred embodiment refers to the use of a prodrug of formula II, more preferably a prodrug of formula Ill, or a prodrug of formula IV in the manufacture of a medicament for the treatment of a disease responsive to TGFβ signaling inhibition.

An additional embodiment of present invention refers to a method for treating a disease responsive to TGFβ signaling inhibition comprising administering an effective amount of a compound of formula I, or a pharmaceutically acceptable salt, or a pharmaceutically acceptable solvate, or a polymorph or a cocrystal thereof, or a composition comprising said compound of formula I, to a subject in need thereof.

Another embodiment of present invention refers to a method for treating a disease responsive to TGFβ signaling inhibition comprising administering an effective amount of a prodrug of a compound of formula I, or a pharmaceutically acceptable salt, or a pharmaceutically acceptable solvate, or a polymorph or a cocrystal thereof, or a composition comprising said prodrug of the compound of formula I, to a subject in need thereof. Preferably, the prodrug of the compound of formula I is a prodrug of formula II, or formula III, or formula IV. A preferred embodiment refers to a method for treating a disease responsive to TGFβ signaling inhibition comprising administering an effective amount of a prodrug of formula II, or formula III, or formula IV, or a pharmaceutically acceptable solvate, or a polymorph or a cocrystal thereof, or a composition comprising said prodrug to a subject in need thereof.

In an embodiment of present invention the disease responsive to the inhibition of the TGFβ signaling pathway is selected from the group consisting of cancer, scleroderma, psoriasis, anemia, sarcopenia, Alzheimer's disease, Marfan syndrome, aneurysm, pulmonary hypertension, osteogenesis imperfecta, idiopathic pulmonary fibrosis, liver fibrosis, cirrhosis, hepatic steatosis, hypertrophic cardiomyopathy, myelofibrosis, neurofibromatosis type I, fibrotic kidney disease, focal segmental glomerulosclerosis, radiation-induced fibrosis, skin fibrosis in systemic sclerosis, diffuse systemic sclerosis, scarring, corneal primary pterygium, fibrosis, uterine leiomyoma, obesity, diabetes, microangiopathy in diabetic retinopathy and nephropathy. In a particular embodiment the scarring is a pathological skin scarring, cutaneous scarring or corneal scarring.

In a particular embodiment, the diseases responsive to the inhibition of the TGFβ signaling pathway also comprise arterial restenosis.

In cancer progression, TGFβ signaling has been implicated in cell proliferation, angiogenesis, epithelial-to-mesenchymal transition, immune infiltration and regulation, metastatic dissemination, and drug resistance. As mentioned previously, the overall effect of the TGFβ signaling pathway seems to strongly promote tumor growth, invasion and metastasis.

In a preferred embodiment the disease responsive to the inhibition of the TGFβ signaling pathway is cancer. In a preferred embodiment, the cancer is a solid tumor. In another preferred embodiment the cancer is not a solid cancer. In an additional preferred embodiment, the cancer is a pediatric cancer.

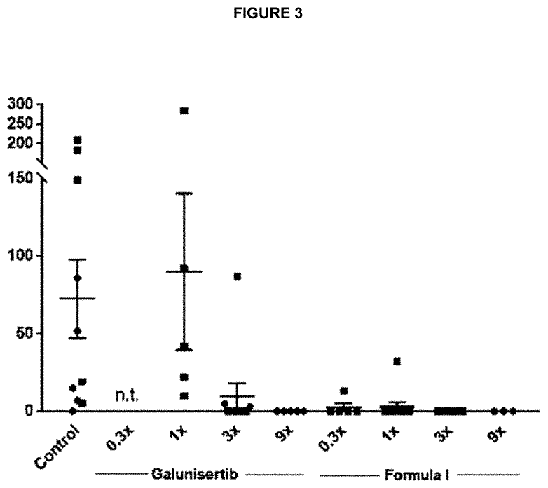

The compound of formula I, as seen in FIG. 3, provided an improved capacity to block liver metastasis formation compared to the reference compound galunisertib, even at one third the equimolar dose of galunisertib.

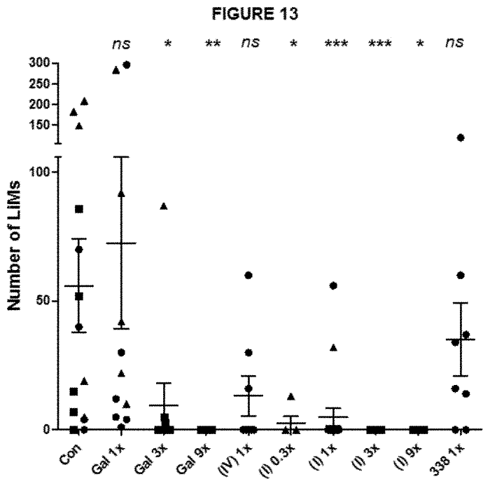

FIG. 13, shows how the compound of formula I exhibits a large improvement of therapeutic activity and blocks liver metastasis formation to a much larger extent than galunisertib and, unexpectedly, also in comparison with reference compound 4-[2-(methyl-pyridin-2-yl) 5,6-dihydro-4H-pyrrolo[1,2-b]pyrazol-3-yl]-quinolin-7-ol (reference compound 338) disclosed by the prior art:

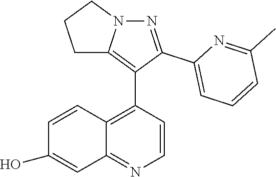 [SMILES: Cc1cccc(-c2nn3c(c2-c2ccnc4cc(O)ccc24)CCC3)n1]

### Reference Compound 338

As seen, the results shown in FIG. 13 are highly unexpected since reference compound 338 is a constitutional isomer of the compound of formula I, having the phenolic hydroxyl in position 7 of the quinoline moiety, whereas the compound of formula I features said phenolic hydroxyl in the 6-position of the quinoline moiety.

In this sense, enzymatic activity inhibition, measured as IC₅₀, of the compound of formula I for ALK5 also confirms the unexpected high potency of the compound of the invention in comparison to that of prior art compounds 337, 338 and galunisertib.

The structure of 4-(2-pydridin-2-yl-5,6-dihydro-4H-pyrrolo[1,2-b]pyrazol-3-yl)-quinolin-7-ol (reference compound 337) differs to that of the compound of formula I in that reference compound 337 does not include a methyl group in the 2-position of the pyridine moiety and features the phenolic hydroxyl in position 7 of the quinoline moiety, and not in the 6-position of the quinoline moiety, as the compound of formula I does.

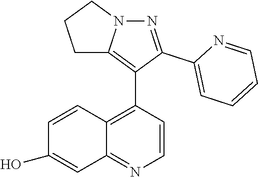 [SMILES: Oc1ccc2c(-c3c(-c4ccccn4)nn4c3CCC4)ccnc2c1]

### Reference Compound 337

As seen in table 2 featured in example 2, the results show that, unexpectedly, the compound of formula (I) is 2.1 times more potent than reference compound 338, and 1.8 times more potent than reference compound 337, knowing that structurally said compounds only differ in the position of the phenolic hydroxyl and in the absence or presence of a methyl group in the pyridine moiety.

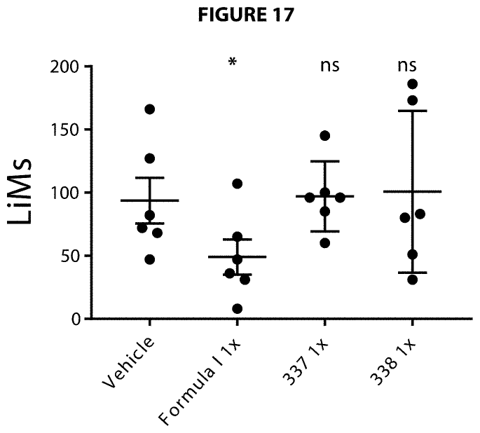

Additional in vivo assays compare the therapeutic effects of compound of formula I with compounds 337 and 338. FIG. 17 shows that the compound of formula I exhibits a large improvement of therapeutic activity and blocks liver metastasis formation to a much larger extent than compound 338, which confirms the results shown in FIG. 13. This experiment also demonstrates that compound of formula (I) exhibit enhanced therapeutic activity compared to compound 337 in these in vivo metastasis assays. Present invention shows, therefore, that the type of substitutions, and position thereof in the core structure, surprisingly provide the unexpected improved potency shown by the compound of formula (I). Moreover, Li et al. (J. Med. Chem. 2008, 51(7): 2302-2306) disclose that electron-deficient and relatively small groups were tolerated at the 6-position, teaching away from the introduction of electron-donating groups such as the hydroxyl featured in the 6-position by the compound of formula (I), resulting instead in a surprisingly strong potency when compared to the prior art constitutional isomer 338, which instead features said hydroxyl in the 7-position.

On the other hand, FIG. 13 also shows that the prodrug of formula IV has up to 10 times lower TGFβ signaling pathway inhibitory capacity in vitro yet provides a similar therapeutic activity in the in vivo metastasis assays than that of the compound of formula I. Therefore, whereas the prodrug of formula III and the prodrug of formula IV have up to 10 times lower TGFβ signaling inhibitory capacity in in vitro assays than compound of formula I, they exhibit similar therapeutic activity in vivo. This result supports that the TGFβ signaling inhibitory capacity of the formula III and the prodrug of formula IV is caged or blocked in vitro but released in vivo.

In a further preferred embodiment, the cancer is a metastatic cancer. Preferably, the metastatic cancer is an overt metastasis or overt metastatic disease.

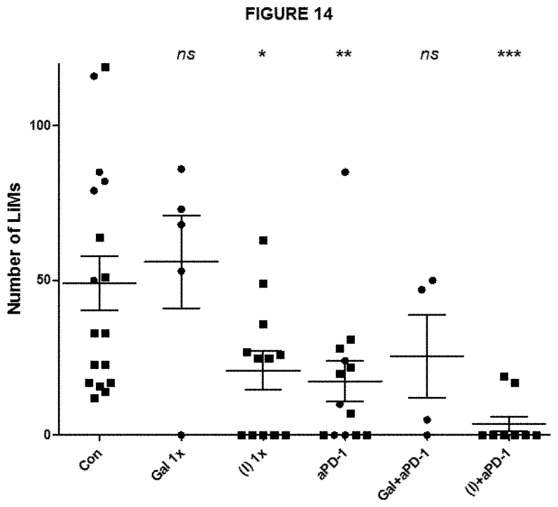

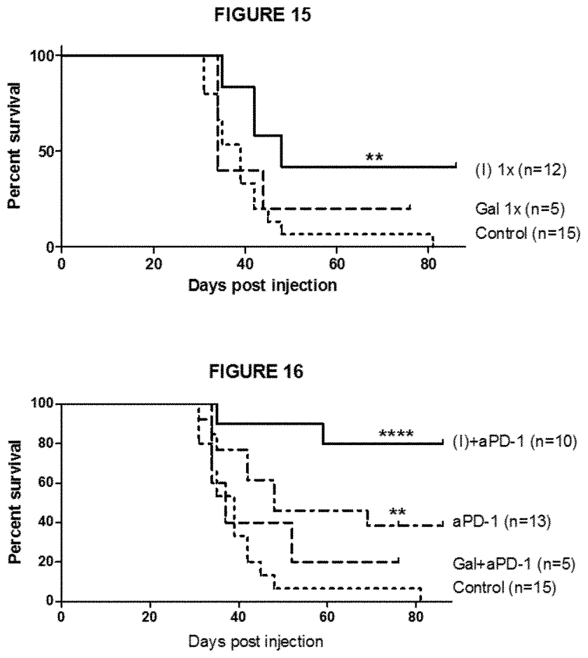

In this sense, FIG. 14 shows how the compound of formula I exerts a very substantial improved activity in an animal model of overt metastatic disease compared to prior art compound galunisertib, when administered at equimolar doses. Moreover, FIG. 15 confirms that the administration of the compound of formula I in said animal model of overt metastatic disease also resulted in a higher survival rate.

On the other hand, the compound of formula I may be administered in combination with other therapies. As mentioned previously, in one embodiment, the compound of formula I, or a prodrug thereof having formula II, or formula Ill, formula IV, or a pharmaceutically acceptable salt, or a pharmaceutically acceptable solvate, or a polymorph or a cocrystal thereof, may be used together with immunotherapy, for example with immunotherapy directed against the immune checkpoint.

Thus, a preferred embodiment refers to a pharmaceutical composition comprising a compound of formula I, or a prodrug thereof having formula II, or formula Ill, formula IV, or a pharmaceutically acceptable salt, or a pharmaceutically acceptable solvate, or a polymorph or a cocrystal thereof, and an immune checkpoint inhibitor, more preferably an anti-PD1 or PDL1 antibody. To this end, FIGS. 14 and 16 show how combination therapy with the compound of formula I and anti-PD-1 antibodies provided a large increase in survival rates and reduced the number of liver metastases in an animal model of overt metastatic disease.

In a preferred embodiment, the disease responsive to the inhibition of the TGFβ signaling pathway is a cancer selected from the group consisting of hematologic cancer; B-cell or T-cell leukemia, non-Hodgkin lymphoma, non-Hodgkin lymphoma B-cell or T-cell types, Burkitt lymphoma, Hodgkin lymphoma, leukemias, lymphoma B-cell or T-cell types, multiple myeloma, brain cancer, cancer of glial lineage of the central nervous system (glioma), gliobastoma, sarcomas, fibrosarcoma, malignant fibrous histiocytoma, Ewing's sarcoma, osteosarcoma, malignant pleural mesothelioma, breast cancer, breast cancer resistant to anti-HER2 therapy, breast carcinoma, breast adenocarcinoma, metastatic breast cancer, gastric and gastroesophageal cancer, gastric carcinoma, gastric adenocarcinoma, colorectal cancer, colon carcinoma, colon adenocarcinoma, rectum cancer, colorectal carcinoma, metastatic colon cancer, pancreatic cancer, pancreas carcinoma, pancreatic adenocarcinoma, renal cell carcinoma, clear cell renal cell carcinoma, liver cancer, liver metastatic cancer, metastatic disease, lung cancer, lung carcinoma, lung adenocarcinoma, non-small-cell lung cancer, small-cell lung cancer, ovarian cancer, ovarian carcinoma, ovarian adenocarcinoma, ovarian carcinoma, endometrial carcinoma, endometrial stromal sarcoma, carcinoma of the uterine cervix, thyroid carcinoma, metastasizing papillary thyroid carcinoma, follicular thyroid carcinoma, bladder carcinoma, urine bladder carcinoma, transitional cell carcinoma of the urinary bladder, prostate cancer, prostate carcinoma, neuroendocrine cancers, squamous cell carcinoma, osteosarcoma, rhabdomyosarcoma, embryonal cancers, neuroblastoma, medulloblastoma, retinoblastoma, nephroblastoma, hepatoblastoma, melanoma and skin cancer.

In a preferred embodiment, the disease responsive to the inhibition of the TGFβ signaling pathway is metastatic disease.

Preferably, the hematologic cancer is leukemia, multiple myeloma or myelodysplastic syndrome. Preferably, the brain cancer is a glioblastoma.

In a preferred embodiment the solid tumor is selected from the group consisting of breast cancer, gastric and gastroesophageal cancer, colorectal cancer, pancreatic cancer, liver cancer, liver metastatic cancer, metastatic disease, lung cancer, ovarian cancer, prostate cancer, neuroendocrine cancers. Preferably, the skin cancer is a melanoma.

In a preferred embodiment the disease responsive to the inhibition of the TGFβ signaling pathway is a cancer selected from the group consisting of leukemia, multiple myeloma, myelodysplastic syndrome, glioblastoma, Ewing's sarcoma, osteosarcoma, malignant pleural mesothelioma, breast cancer, gastric and gastroesophageal cancer, colorectal cancer, pancreatic cancer, liver cancer, liver metastatic cancer, metastatic disease, lung cancer, ovarian cancer, prostate cancer, neuroendocrine cancers and melanoma.

Additionally, to the prodrugs of formula II, which contain an acetovanillone-derived fragment, present invention also discloses other acetovanillone-derived prodrugs. Accordingly, the acetovanillone-derived fragment of the prodrug of formula II, (AcV):

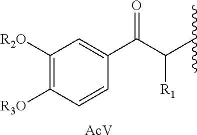

can also be used to obtain prodrugs of other drugs different from the compound of formula I, whereby said drugs must comprise one hydroxyl group. In this sense, said drug comprising one hydroxyl group, of structure R—OH can react with a compound comprising the acetovanillone fragment AcV-X, forming the ether bond of a prodrug AcV-OR of formula V as seen in Scheme VI:

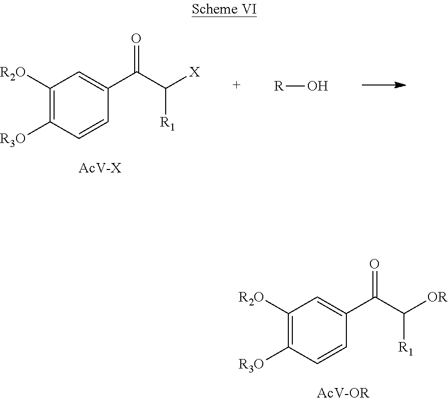

When the beta-keto ether bond is hydrolyzed in the presence of a beta-etherase enzyme or by liver enzymes, such as the cytochrome P450, the drug comprising one hydroxyl group R—OH is released. Examples of medicaments comprising one hydroxyl, of formula R—OH are, without not being limited to, 4-hydroxytamoxifen or 7-ethyl-10-hydroxy-camptothecin (SN-38, CAS No: 86639-52-3, active metabolite of irinotecan).

Example 6 shows how LigF, beta-etherase enzyme is capable of hydrolyzing the ether bond cleaving the acetovanillone fragment in a model experiment carried out with the acetovanillone ether of 4-methylumbelliferone and with the acetovanillone ether of 4-hydroxytamoxifen.

In this sense, present invention also refers, in general, to prodrugs of formula V of a drug comprising one hydroxyl of formula R—OH, wherein said prodrugs comprise an acetovanillone-derived fragment:

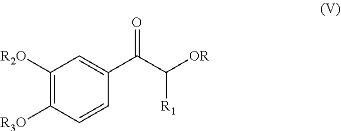

wherein
R—OH is a drug comprising one hydroxyl;
R₁ is selected from the group consisting of H, alkyl, cycloalkyl, haloalkyl, hydroxyalkyl, aminoalkyl, alkanoylamino, alkoxy, aryl, alkylaryl, arylalkyl, amino, N-alkylamino and N,N-dialkylamino; and
R₂ and R₃ are each independently selected from the group consisting of H, alkyl and haloalkyl.

In one embodiment R₁ is selected from the group consisting of H, alkyl, cycloalkyl, haloalkyl, hydroxyalkyl, aminoalkyl, alkanoylamino, alkoxy, aryl, alkylaryl, arylalkyl, amino, N-alkylamino and N,N-dialkylamino; R₂ is methyl and R₃ is H. In another embodiment R₁ is selected from the group consisting of H, alkyl, haloalkyl, aminoalkyl and hydroxyalkyl; R₂ is methyl and R₃ is H.

One embodiment refers to a pharmaceutical composition comprising an effective amount of a prodrug of formula V, and at least one pharmaceutically acceptable excipient.

One embodiment of present invention refers to a prodrug of 4-hydroxytamoxifen of formula VI:

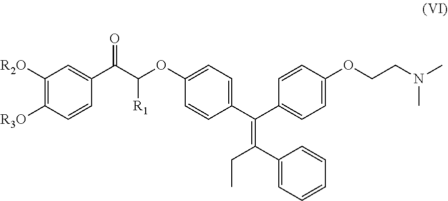

wherein
R₁ is selected from the group consisting of H, alkyl, cycloalkyl, haloalkyl, hydroxyalkyl, aminoalkyl, alkanoylamino, alkoxy, aryl, alkylaryl, arylalkyl, amino, N-alkylamino and N,N-dialkylamino; and
R₂ and R₃ are each independently selected from the group consisting of H, alkyl and haloalkyl.

One embodiment refers to a prodrug of 4-hydroxytamoxifen, of formula VI, wherein R₁ is selected from the group consisting of H, alkyl, cycloalkyl, haloalkyl, hydroxyalkyl, aminoalkyl, alkanoylamino, alkoxy, aryl, alkylaryl, arylalkyl, amino, N-alkylamino and N,N-dialkylamino; R₂ is methyl and R₃ is H. In another embodiment R₁ is selected from the group consisting of H, alkyl, haloalkyl, aminoalkyl and hydroxyalkyl; R₂ is methyl and R₃ is H.

Although one isomer is depicted, both isomers Z and E, and mixtures thereof, are included in the prodrug of formula VI. In an embodiment the prodrug of formula VI is the Z-isomer. In another embodiment the prodrug of formula VI is the E-isomer. In a further embodiment the prodrug of formula VI is a mixture of the Z-isomer and the E-isomer. In particular, the mixture comprises a mixture of both isomers in a 50:50, 60:40, 70:30, 80:20, 90:10, 95:5, 99:1 ratio or any other ratio.

In a preferred embodiment R₁ is H, R₂ is methyl and R₃ is H, and the prodrug of 4-hydroxytamoxifen of formula VI, is a prodrug of formula VII:

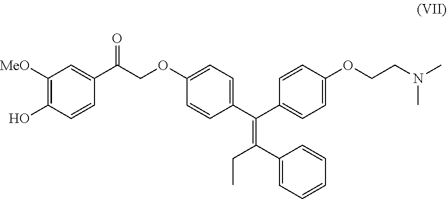 [SMILES: CC/C(=C(/c1ccc(OCCN(C)C)cc1)c1ccc(OCC(=O)c2ccc(O)c(OC)c2)cc1)c1ccccc1]

Although one isomer is depicted, both isomers Z and E, and mixtures thereof, are included in the prodrug of formula VII. In an embodiment the prodrug of formula VII is the Z-isomer. In another embodiment the prodrug of formula VII is the E-isomer. In a further embodiment the prodrug of formula VII is a mixture of the Z-isomer and the E-isomer. In particular, the mixture comprises a mixture of both isomers in a 50:50, 60:40, 70:30, 80:20, 90:10, 95:5, 99:1 ratio or any other ratio.

One embodiment refers to a pharmaceutical composition comprising an effective amount of a prodrug of formula VI, or formula VII, and at least one pharmaceutically acceptable excipient.

The prodrugs of formula VI, or formula VII, are much less active, compared to 4-hydroxytamoxifen (4-OHT).

4-hydroxytamoxifen is an estrogen receptor (ER) antagonist used in the treatment of cancer, preferably, but not exclusively, in the prevention and/or treatment of breast cancer. In addition, it is a strong agonist of mutated versions of the ER (especially the ER^{T2} variant) which can be used to trigger activation of fusion proteins, such as Cre-ER^{T} and Cre-ER^{T2}, (T=for inducible by Tamoxifen), that have their main activity in the cell nucleus as described in patent EP1692936B1.

In particular the Cre-ER^{T} fusion protein comprises at least the portion of the nuclear estrogen receptor having a ligand binding activity. The ligand binding activity of the nuclear receptor, or one of its fragments, has at least one mutation chosen from (1) a mutation (G521 R) glycine to arginine at position 521 of the human nuclear estrogen receptor sequence or of a natural variant of this sequence; (2) a mutation (G400V) glycine to valine at position 400 of the human nuclear estrogen receptor sequence or of a natural variant of this sequence; and (3) a mutation (methionine-leucine) to (alanine-alanine) situated at position 543-544 (M543A/L544A mutation) of the human nuclear estrogen receptor sequence or of a natural variant of this sequence.

As described in patent EP1692936B1, Cre-ER^{T2} is a triple mutant protein G400V/M543A/L544A and Cre-ERT fusion protein which carries the G521 R mutation.

In the absence of 4-OHT, the fusion protein is sequestered in the cytoplasm and thereby inactive; yet upon binding of 4-OHT to ER^{T2}, this shuttles the fusion protein into the nucleus. When ER^{T2} is fused to a Cre (Can Recombine) protein, and when using specific genetic regions that are flanked by Cre recognition sites (loxP sites), this allows the use of 4-OHT to activate or delete a gene in cells at a specific location. In this sense, it is possible to locally cleave the prodrug liberating 4-hydroxytamoxifen (4-OHT) only around cells that have encoded a specific enzyme as seen in example 6.

The prodrug of formula VII was synthesized as described in example 5 departing from known compounds 4,4′-(1-phenylbut-1-ene-1,2-diyl)diphenol (compound 5-43) and acetovanillone. In this sense, Scheme VII describes the synthetic path used:

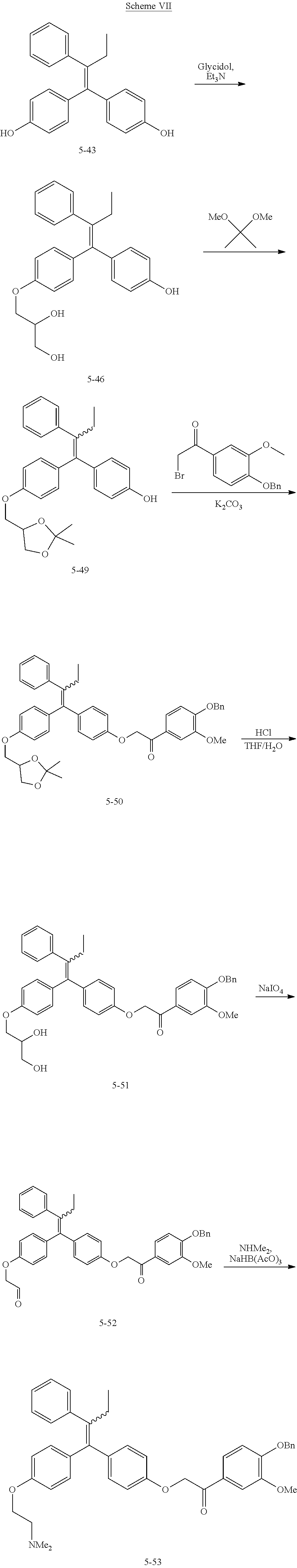

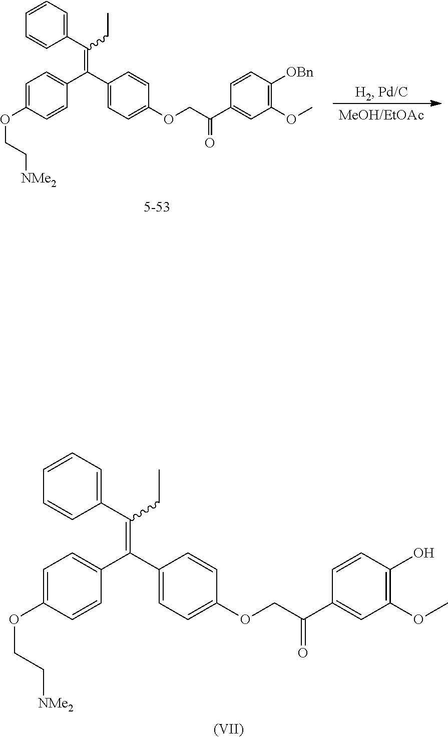 [SMILES: CCC(=C(c1ccc(OCCN(C)C)cc1)c1ccc(OCC(=O)c2ccc(OCc3ccccc3)c(OC)c2)cc1)c1ccccc1 ; "H2, Pd/C" ; "MeOH/EtOAc" ; CCC(=C(c1ccc(OCCN(C)C)cc1)c1ccc(OCC(=O)c2ccc(O)c(OC)c2)cc1)c1ccccc1 ; "(VII)"]

In this sense, the synthesis departs from compound 5-43 (CAS No: 91221-46-4) a dihydroxy precursor of tamoxifen, from 4,4′-dihydroxybenzophenone and propiophenone. The hydroxyl group, corresponding to the position featuring the N. N-dimethyl aminoethyl ether group of 4-hydroxytamoxifen, is first converted to an ether with glycidol. The diol subsequently protected with 2,2-dimethoxypropane, to couple the acetovanillone-derived fragment by alkylation in the second hydroxyl. Further steps include deprotection of the diol group, which is then oxidized to aldehyde and converted subsequently to amine via reductive amination.

The prodrug of formula VII is inactive, but in the presence of a beta-etherase enzyme can release 4-OHT. which is able to activate or delete a gene in cells at a specific location as shown in example 6, where using a Cre-ER^{T2} enzyme, or acts as an anti-cancer drug, antagonizing the wildtype estrogen receptor.

In this manner 4-hydroxytamoxifen is only liberated in cells that have encoded a beta-etherase enzyme such as LigF-which was encoded in the DNA of tumor cells as described in example 6. The enzymatic activity of LigF would liberate 4-hydroxytamoxifen from the prodrug of formula VI, or from the prodrug of formula VII.

Alternatively, the prodrug of formula VI, or the prodrug of formula VII, can be cleaved by enzymes such as the cytochrome P450 enzyme family (mainly present in the liver), potentially at lower efficiency than LigF. Example 7 describes a mouse treatment, wherein the liver was found to be able to actively cleave the prodrug and undergo gene recombination in a dose-dependent way, indicating that this prodrug strategy can be used to target the liver specifically with any compound of formula V, including an acetovanillone beta-ether, and in particular a prodrug of formula VI. Gene recombination in peripheral tissues was lower when compared to the liver, indicating the possibility to target the liver.

In this sense the prodrug of formula VI, or of formula VII, may be used as a research tool in biomedicine, genetics, developmental biology, cell biology, stem cell biology, or a related field.

Accordingly, herein described is a method for performing a recombination of one or more genes, or of one or more sequences of interest in a mouse, wherein said one or more genes or sequences belong to said mouse native genome, in a group of cells of said mouse expressing a Cre-ER^{T2} fusion protein in the presence of 4-hydroxytamoxifen, said method comprising:
a) providing a mouse comprising one or more recognition sites for the Cre recombinase protein, said recognition sites being inserted into said DNA sequences of interest which are located in one or more of the chromosomes of the mouse genome; and a Cre-ER^{T2} fusion protein having substantially no recombinase activity in the absence of 4-hydroxytamoxifen or in the presence of a natural estrogen, the recombinase activity being induced by small quantities of 4-hydroxitamoxifen;
b) contacting said mouse with a prodrug of formula VI;
c) when recombination is desired, contacting at least part of the cells of the mouse with a beta-etherase enzyme for reacting with the compound of formula VI, liberating 4-hydroxytamoxifen and induce the recombinase activity of said Cre-ER^{T2} fusion protein, and obtaining recombination of said one or more genes or sequences of interest in the specific area local to the presence of the beta-etherase enzyme, being the source of liberated 4-hydroxytamoxifen; and

whereas said recombination does not occur substantially in said cells expressing the Cre-ER^{T2} fusion protein in the absence of 4-hydroxytamoxifen, or in the presence of a natural estrogen.

In an embodiment, the beta-etherase enzyme is LigF and the prodrug of formula VI has formula VII. For the purposes of present invention, the Cre-ER^{T2} fusion protein having substantially no recombinase activity in the absence of 4-hydroxytamoxifen or in the presence of a natural estrogen, indicates that said fusion protein has less than 10% recombinase activity or less than 5%, or even less than 1%. In an analogous manner, the recombination does not substantially occur in cells expressing the Cre-ER^{T2} fusion protein in the absence of 4-hydroxytamoxifen, or in the presence of a natural estrogen, also indicates that the recombination occurs in less than 10%, 5% or even 1% of the cases in absence of 4-hydroxytamoxifen, or in the presence of a natural estrogen.

Another embodiment of present invention refers to a prodrug of formula VI, or formula VII, or pharmaceutical compositions thereof, for use as a medicament. A further embodiment refers to a prodrug of formula VI, preferably a prodrug of formula VII, for use in the treatment of cancer. Preferably a cancer selected from the group consisting of hematologic cancer; B-cell or T-cell leukemia, non-Hodgkin lymphoma, non-Hodgkin lymphoma B-cell or T-cell types, Burkitt lymphoma, Hodgkin lymphoma, leukemias, lymphoma B-cell or T-cell types, multiple myeloma, brain cancer, cancer of glial lineage of the central nervous system (glioma), sarcomas, fibrosarcoma, malignant fibrous histiocytoma, Ewing's sarcoma, osteosarcoma, malignant pleural mesothelioma, breast cancer, breast cancer resistant to anti-HER2 therapy, breast carcinoma, breast adenocarcinoma, gastric and gastroesophageal cancer, gastric carcinoma, gastric adenocarcinoma, colorectal cancer, colon carcinoma, colon adenocarcinoma, rectum cancer, colorectal carcinoma, metastatic colon cancer, pancreatic cancer, pancreas carcinoma, pancreatic adenocarcinoma, renal cell carcinoma, clear cell renal cell carcinoma, liver cancer, lung cancer, lung carcinoma, lung adenocarcinoma, non-small-cell lung cancer, small-cell lung cancer, ovarian cancer, ovarian carcinoma, ovarian adenocarcinoma, ovarian carcinoma, endometrial carcinoma, endometrial stromal sarcoma, carcinoma of the uterine cervix, thyroid carcinoma, metastasizing papillary thyroid carcinoma, follicular thyroid carcinoma, bladder carcinoma, urine bladder carcinoma, transitional cell carcinoma of the urinary bladder, prostate cancer, prostate carcinoma, neuroendocrine cancers, squamous cell carcinoma, osteosarcoma, rhabdomyosarcoma, embryonal cancers, neuroblastoma, medulloblastoma, retinoblastoma, nephroblastoma, hepatoblastoma, melanoma and skin cancer.

Preferably, the hematologic cancers is leukemia, multiple myeloma or myelodysplastic syndrome. Preferably, the brain cancer is a glioblastoma.

In a preferred embodiment the solid tumor is selected from the group consisting of breast cancer, gastric and gastroesophageal cancer, colorectal cancer, pancreatic cancer, liver cancer, lung cancer, ovarian cancer, prostate cancer, neuroendocrine cancers

Preferably, the skin cancer is a melanoma.

An additional embodiment of present invention refers to a prodrug of 7-ethyl-10-hydroxy-camptothecin, of formula VIII:

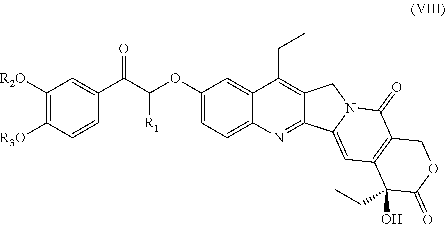

wherein
R₁ is selected from the group consisting of H, alkyl, cycloalkyl, haloalkyl, hydroxyalkyl, aminoalkyl, alkanoylamino, alkoxy, aryl, alkylaryl, arylalkyl, amino, N-alkylamino and N,N-dialkylamino; and
R₂ and R₃ are each independently selected from the group consisting of H, alkyl and haloalkyl.

One embodiment refers to a prodrug of 7-ethyl-10-hydroxy-camptothecin of formula VIII, wherein R₁ is selected from the group consisting of H, alkyl, cycloalkyl, haloalkyl, hydroxyalkyl, aminoalkyl, alkanoylamino, alkoxy, aryl, alkylaryl, arylalkyl, amino, N-alkylamino and N,N-dialkylamino; R₂ is methyl and R₃ is H. In another embodiment R₁ is selected from the group consisting of H, alkyl, haloalkyl, aminoalkyl and hydroxyalkyl; R₂ is methyl and R₃ is H.

In a preferred embodiment of the prodrug of formula VII,I R₁ is H, R₂ is methyl and R₃ is H, and the prodrug of 7-ethyl-10-hydroxy-camptothecin, is a prodrug of formula IX:

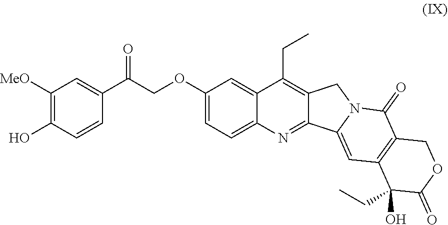 [SMILES: CCc1c2c(nc3ccc(OCC(=O)c4ccc(O)c(OC)c4)cc13)-c1cc3c(c(=O)n1C2)COC(=O)[C@]3(O)CC]

One embodiment refers to a pharmaceutical composition comprising an effective amount of a prodrug of formula VIII, or of formula IX, and at least one pharmaceutically acceptable excipient.

An embodiment described refers to a prodrug of 7-ethyl-10-hydroxy-camptothecin of formula VIII, or of formula IX, or pharmaceutical compositions thereof, for use as a medicament, in particular for use in the treatment of cancer. Preferably a cancer selected from the group consisting of hematologic cancer; B-cell or T-cell leukemia, non-Hodgkin lymphoma, non-Hodgkin lymphoma B-cell or T-cell types, Burkitt lymphoma, Hodgkin lymphoma, leukemias, lymphoma B-cell or T-cell types, multiple myeloma, brain cancer, cancer of glial lineage of the central nervous system (glioma), sarcomas, fibrosarcoma, malignant fibrous histiocytoma, Ewing's sarcoma, osteosarcoma, malignant pleural mesothelioma, breast cancer, breast cancer resistant to anti-HER2 therapy, breast carcinoma, breast adenocarcinoma, gastric and gastroesophageal cancer, gastric carcinoma, gastric adenocarcinoma, colorectal cancer, colon carcinoma, colon adenocarcinoma, rectum cancer, colorectal carcinoma, metastatic colon cancer, pancreatic cancer, pancreas carcinoma, pancreatic adenocarcinoma, renal cell carcinoma, clear cell renal cell carcinoma, liver cancer, lung cancer, lung carcinoma, lung adenocarcinoma, non-small-cell lung cancer, small-cell lung cancer, ovarian cancer, ovarian carcinoma, ovarian adenocarcinoma, ovarian carcinoma, endometrial carcinoma, endometrial stromal sarcoma, carcinoma of the uterine cervix, thyroid carcinoma, metastasizing papillary thyroid carcinoma, follicular thyroid carcinoma, bladder carcinoma, urine bladder carcinoma, transitional cell carcinoma of the urinary bladder, prostate cancer, prostate carcinoma, neuroendocrine cancers, squamous cell carcinoma, osteosarcoma, rhabdomyosarcoma, embryonal cancers, neuroblastoma, medulloblastoma, retinoblastoma, nephroblastoma, hepatoblastoma, melanoma and skin cancer.

Preferably, the hematologic cancers is leukemia, multiple myeloma or myelodysplastic syndrome. Preferably, the brain cancer is a glioblastoma.

In a preferred embodiment the solid tumor is selected from the group consisting of breast cancer, gastric and gastroesophageal cancer, colorectal cancer, pancreatic cancer, liver cancer, lung cancer, ovarian cancer, prostate cancer, neuroendocrine cancers Preferably, the skin cancer is a melanoma.

The acetovanillone-derived fragment of the prodrugs of formula V, and in particular of the prodrugs of formula VI, VII, VIII and IX, can be oxidized and cleaved by liver enzymes, such as the cytochrome P450 enzymes, which hydrolyze the ether bond delivering the drug comprising one hydroxyl R—OH, which is 4-hydroxytamoxifen in the case of the prodrugs of formula VI and VII, and 7-ethyl-10-hydroxy-camptothecin in the case of the prodrugs of formula VIII and IX. On the other hand, the ether bond can also be hydrolyzed by specific beta-etherases from non-mammalian sources, such as bacterium Sphingobium sp. strain SYK-6 enzymes LigE and LigF, or fungus Dichomitus squalens Ds-GST1, which can be administered in conjunction with the prodrug of formula V, and in particular with a prodrug of formula VI, VII, VIII or IX.

In a particular embodiment the beta-etherase enzyme is administered in cells expressing said beta-etherase enzyme, as cell therapy, for instance, adoptive T-cell transfer or dendritic cell vaccination. Alternatively, in another particular embodiment the beta-etherase enzyme is administered coupled to an antibody which is tumor or stroma specific, optionally with glutathione. Certain tissues such as the lung lining comprise high extracellular levels of gluthatione, and accordingly the administration of gluthatione is not required.

For the purposes of present description, the term “active compound” means a chemical entity or active principle which exerts therapeutic effects when administered to a human or an animal.

Typical compositions include said compounds of the invention in association with at least one pharmaceutically acceptable excipient, which may be a carrier or a diluent, by a way of example. Such compositions can be in the form of a capsule, sachet, paper or other container.

In making the compositions, conventional techniques for the preparation of pharmaceutical compositions may be used. For example, the compound of interest will usually be mixed with a carrier, or diluted by a carrier, or enclosed within a carrier that may be in the form of an ampoule, capsule, sachet, paper, or other container. When the carrier serves as a diluent, it may be solid, semi-solid, or liquid material that acts as a vehicle, excipient, or medium for the active compound. The compound of interest can be adsorbed on a granular solid container for example in a sachet. Some examples of suitable carriers are water, salt solutions, alcohols, polyethylene glycols, polyhydroxyethoxylated castor oil, peanut oil, olive oil, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavoring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or coloring substances and the like, which do not deleteriously react with the active compounds.

One preferred embodiment of the present invention refers to the route of administration, that may be any route which effectively transports the compound of interest to the appropriate or desired site of action, such as oral, buccal, nasal, topical, pulmonary, transdermal or parenteral, e.g. rectal, subcutaneous, intravenous, intraurethral, intramuscular, intranasal, ophthalmic solution or an ointment.

For nasal administration, the preparation may contain the compound of interest dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine), or cyclodextrin, or preservatives such as parabens.

To prepare topical formulations, the compound interest is placed in a dermatological vehicle as is known in the art. The amount of the compound of interest to be administered and the compound's concentration in the topical formulations depend upon the vehicle, delivery system or device selected, the clinical condition of the patient, the side effects and the stability of the compound in the formulation. Thus, the physician employs the appropriate preparation containing the appropriate concentration of the compound of interest and selects the amount of formulation administered, depending upon clinical experience with the patient in question or with similar patients.

For ophthalmic applications, the compound of interest is formulated into solutions, suspensions, and ointments appropriate for use in the eye. The concentrations are usually as discussed above for local preparations.

For oral administration, either solid or fluid unit dosage forms can be prepared. For preparing solid compositions such as tablets, the compound of interest is mixed into formulations with conventional ingredients such as talc, magnesium stearate, dicalcium phosphate, magnesium aluminum silicate, calcium sulfate, starch, lactose, acacia, methylcellulose, and functionally similar materials as pharmaceutical diluents or carriers.

Capsules are prepared by mixing the compound of interest with an inert pharmaceutical diluent and filling the mixture into a hard gelatin capsule of appropriate size. Soft gelatin capsules are prepared by machine encapsulation of slurry of the compound of interest with an acceptable vegetable oil, light liquid petrolatum or other inert oil. Fluid unit dosage forms for oral administration such as syrups, elixirs and suspensions can be prepared. The water-soluble forms can be dissolved in an aqueous vehicle together with sugar, aromatic flavoring agents and preservatives to form syrup. An elixir is prepared by using a hydroalcoholic (e.g. ethanol) vehicle with suitable sweeteners such as sugar and saccharin, together with an aromatic flavoring agent. Suspensions can be prepared with an aqueous vehicle with the aid of a suspending agent such as acacia, tragacanthin, methylcellulose and the like.

Appropriate formulations for parenteral use are apparent to the practitioner of ordinary skill, such as the use of suitable injectable solutions or suspensions. The formulation, which is sterile, is suitable for various topical or parenteral routes including intra-dermal, intramuscular, intravascular, and subcutaneous.

In addition to the compound of interest, the compositions may include, depending on the formulation and mode of delivery desired, pharmaceutically-acceptable, non-toxic carriers or diluents, which include vehicles commonly used to form pharmaceutical compositions for animal or human administration. The diluent is selected so as not to unduly affect the biological activity of the combination.

Examples of such diluents that are especially useful for injectable formulations are water, the various saline, organic or inorganic salt solutions, Ringer's solution, dextrose solution, and Hank's solution. In addition, the pharmaceutical composition or formulation may include additives such as other carriers; adjuvants; or non-toxic, non-therapeutic, non-immunogenic stabilizers and the like.

Furthermore, excipients can be included in the formulation. Examples include cosolvents, surfactants, oils, humectants, emollients, preservatives, stabilizers and antioxidants. Any pharmacologically acceptable buffer may be used, e.g. tris or phosphate buffers. Effective amounts of diluents, additives, and excipients are those amounts that are effective to obtain a pharmaceutically acceptable formulation in terms of solubility or biological activity.

The compound of interest may be incorporated into a microsphere. The compound of interest can be loaded into albumin microspheres, from which it is possible to recover such microspheres in a dry powder for nasal administration. Other materials suitable for the preparation of microspheres include agar, alginate, chitosan, starch, hydroxyethyl starch, albumin, agarose, dextran, hyaluronic acid, gelatin, collagen, and casein. The microspheres can be produced by various processes known to the person skilled in the art such as a spray drying process or an emulsification process.

For example, albumin microspheres can be prepared by adding rabbit serum albumin in phosphate buffer to olive oil with stirring to produce water in oil emulsion. Glutaraldehyde solution is then added to the emulsion and the emulsion stirred to cross-link the albumin. The microspheres can then be isolated by centrifugation, the oil removed, and the spheres washed, e.g. with petroleum ether followed by ethanol. Finally, the microspheres can be sieved and collected and dried by filtration.

Starch microspheres can be prepared by adding a warm aqueous starch solution, e.g. of potato starch, to a heated solution of polyethylene glycol in water with stirring to form an emulsion. When the two-phase system has formed (with the starch solution as the inner phase) the mixture is then cooled to room temperature under continued stirring whereupon the inner phase is converted into gel particles. These particles are then filtered off at room temperature and slurred in a solvent such as ethanol, after which the particles are again filtered off and laid to dry in air. The microspheres can be hardened by well-known cross-linking procedures such as heat treatment or by using chemical cross-linking agents. Suitable agents include dialdehydes, including glyoxal, malondialdehyde, succinicaldehyde, adipaldehyde, glutaraldehyde and phthalaldehyde, diketones such as butadione, epichlorohydrin, polyphosphate, and borate. Dialdehydes are used to cross-link proteins such as albumin by interaction with amino groups, and diketones form Schiff bases with amino groups. Epichlorohydrin activates compounds with nucleophiles such as amino or hydroxyl to an epoxide derivative.

Another preferred embodiment of the invention is the dosage scheme. The term “unit dosage form” refers to physically discrete units suitable as unitary dosages for subjects, e.g. mammalian subjects, e.g. humans, dogs, cats, and rodents, each unit containing a predetermined quantity of active material calculated to produce the desired pharmaceutical effect in association with the required pharmaceutical diluent, carrier or vehicle. The specifications for the unit dosage forms of this invention are dictated by and dependent on (a) the unique characteristics of the active material and the particular effect to be achieved and (b) the limitations inherent in the art of compounding such an active material for use in humans and animals. Examples of unit dosage forms are tablets, capsules, pills, powder packets, wafers, suppositories, granules, cachets, teaspoonfuls, tablespoonfuls, dropperfuls, ampoules, vials, aerosols with metered discharges, segregated multiples of any of the foregoing, and other forms as herein described. The compositions can be included in kits, which can contain one or more unit dosage forms of the composition and instructions for use to treat one or more of the disorders described herein.

Slow or extended-release delivery systems, including any of a number of biopolymers (biological-based systems), systems employing liposomes, colloids, resins, and other polymeric delivery systems or compartmentalized reservoirs, can be utilized with the compositions described herein to provide a continuous or long-term source of therapeutic compound. Such slow release systems are applicable to formulations for delivery via topical, intraocular, oral, and parenteral routes.

An effective amount of the compound of interest is employed in treatment. The dosage of compounds used in accordance with the invention varies depending on the compound and the condition being treated for example the age, weight, and clinical condition of the recipient patient. Other factors include: the route of administration, the patient, the patient's medical history, the severity of the disease process, and the potency of the particular compound. The dose should be sufficient to ameliorate symptoms or signs of the disease treated without producing unacceptable toxicity to the patient. In general, an effective amount of the compound is that which provides either subjective relief of symptoms or an objectively identifiable improvement as noted by the clinician or other qualified observer.

### EXAMPLES

The examples of present invention described below aim to illustrate some of the embodiments disclosed without limiting its scope of protection. Additionally, reference examples are included, referring to embodiments disclosed in present description but not included in scope of the claims. All experiments with mouse models were approved by the Animal Care and Use Committee of Barcelona Science Park (CEEA-PCB) and the Catalan government. Mice are maintained in specific-pathogen-free (SPF) facility with a 12-hour light-dark cycle and fed with standard diet and water ad lib. All mice were closely monitored for animal welfare.

General information on compound dosing in mouse experiments: The molecular weights of Galunisertib (LY2157299 monohydrate), compounds of formula I and formula IV, and compounds 338 and 337 are: 387.4; 342.4; 536.7; 342.4; 328.4 respectively. Therefore, the corresponding molecular weight ratios are: 1:0.88:1.39:0.88:0.85. All mice treatments described in the figures below and in examples are calculated using as a reference the 1× dose for galunisertib which corresponds to 160 mg/kg/day divided over 2 oral administrations (morning and late afternoon), i.e. 80 mg/kg b.i.d. Therefore, the 1× molar equivalent doses of compound of formula I is 70.8 mg/kg b.i.d., of compound of formula IV is 111.2 mg/kg b.i.d, of compound 338 is 70.8 mg/kg b.i.d. and of compound 337 is 68.0 mg/kg b.i.d.

Prior art compounds 337 (CAS No. 476474-11-0) and 338 (CAS No. 476477-67-5) were prepared according to the patent WO2005094833 (both 338 and 337) and J. Med Chem. 2008, 51, 2302 (only described 337).

### Example 1: Synthesis of Compounds of Formula I, III and IV

All reactions were carried out under an inert atmosphere (N₂) unless otherwise stated. Anhydrous THF, DCM and diethyl ether were taken from the Innovative Technology Inc. Puresolv Solvent Purification System (SPS). Other anhydrous solvents were purchased and used without any further purification or drying.

All experiments were monitored by analytical thin-layer chromatography (TLC) using silica gel TLC-aluminum sheets (Merck 60 F254). The results were visualized under a UV lamp (either 254 or 365 nm) and revealed using a KMnO4 stain when necessary.

Flash column chromatography was performed on either a CombiFlash® (Teledyne Isco), or on the Puriflash 430 (Interchim) unless otherwise stated. The mobile phase was either a gradient of hexane/ethyl acetate, or a gradient of dichloromethane/methanol. In some cases where a basic nitrogen was present on the molecule being purified, the column was preconditioned with a 2.5% triethylamine solution in hexanes.

Samples for NMR were dissolved in either CDCl₃, CD₃OD, DMSO or D₂O.

¹H and ¹³C spectra were referenced to residual solvent peaks and/or tetramethylsilane. The coupling constants were measured in hertz (Hz). All IR spectra were performed using the film method on a NaCl disc and recorded on a Thermo Nicolet Nexus Ft-IR Fourier transform spectrometer.

1.1. Synthesis of the Compound of Formula I from p-Bromoaniline—Route (d):

Compound of formula I was synthetized from p-Bromoaniline, including a last step where the bromo was converted into the phenol:

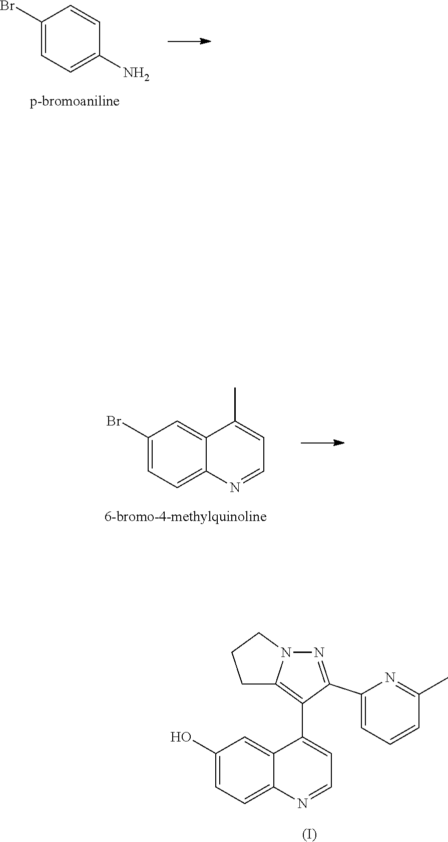

### 6-bromo-4-methylquinoline

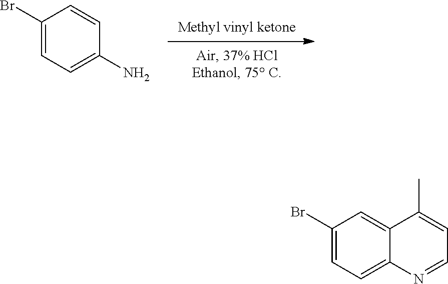

To a stirred solution of 4-bromoaniline (20 g, 117 mmol) and chloranil (34.7 g, 141 mmol, 1.2 equiv.) in ethanol (200 mL, 10 volumes) at rt was added 37% HCl (29.3 mL, 351 mmol, 3 equiv.) over five minutes. After stirring for five minutes at room temperature the reaction mixture was heated to 75° C. where methyl vinyl ketone (15.45 mL, 190.5 mmol, 1.5 equiv.) diluted in ethanol (15 mL, 1 volume) was added to the reaction mixture over thirty minutes. After four hours, no starting material was observed by TLC. The reaction was cooled to 60° C. where it was diluted with THF (165 mL, 11 volumes), stirred for an hour at 60° C. and then left to cool to room temperature overnight. The suspension was filtered through a sintered Büchner funnel. The residue was rinsed with THF (2×45 mL, 6 volumes in total) and then dried at 50° C. overnight in a vacuum oven to give the title compound (21.1 g, 81%) as a brown solid.

¹H NMR (400 MHz, CDCl3) δ 8.78 (d, J=4.4 Hz, 1H), 8.16-8.14 (m, 1H), 7.97 (d, J=9.0 Hz, 1H), 7.77 (dd, J=9.0, 2.2 Hz, 1H), 7.25 (dd, J=4.4, 0.9 Hz, 1H), 2.68 (d, J=0.9 Hz, 3H).

¹³C NMR (101 MHz, DMSO) δ 145.76, 138.20, 135.96, 133.04, 129.21, 127.79, 125.12, 123.25, 122.18, 19.24.

### 6-bromo-4-(2-(6-methylpyridin-2-yl)-5,6-dihydro-4H-pyrrolo[1,2-b]pyrazol-3-yl)quinoline

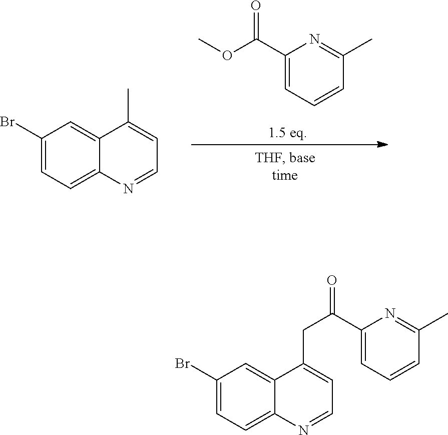

To a stirred solution of 6-bromo-4-methylquinoline (3.70 g, 14.31 mmol) in THF (60 mL) at −78° C. was added 1M NaHMDS (57 mL, 57 mmol, 4 equiv.). The reaction mixture was stirred for 3 hrs followed by the addition of methyl 6-methylpicolinate (2.60 g, 17.17 mmol, 1.2 equiv.) diluted in THF (7.4 mL, 2 volumes) dropwise over 30 minutes. The reaction was left to warm to room temperature overnight and then cooled to 0° C. and taken to pH 1 with 3M HCl. It was allowed to warm to room temperature over one hour and then taken to pH 8 with solid NaHCO₃. The mixture was diluted with water (50 mL) and extracted with EtOAc (1×100 mL) followed by CHCl₃ (1×100 mL). The combined organic extracts were dried (MgSO₄), evaporated and the residue was recrystallised from MeOH (30 mL, 8 volumes) to give 2-(6-bromoquinolin-4-yl)-1-(6-methylpyridin-2-yl)ethan-1-one (2.57 g, 53%) as an off-white solid.

¹H NMR (400 MHz, cdcl3) δ 8.84 (d, J=4.4 Hz, 1H), 8.36 (d, J=2.1 Hz, 1H), 7.97 (d, J=9.0 Hz, 1H), 7.86 (d, J=7.7 Hz, 0H), 7.75 (dd, J=9.0, 2.2 Hz, 1H), 7.73 (t, J=7.7 Hz, 1H), 7.43 (d, J=4.4 Hz, 1H), 7.38 (d, J=8.6 Hz, 1H), 4.96 (s, 1H), 2.70 (s, 2H).

¹³C NMR (101 MHz, cdcl3) δ 197.87, 158.43, 152.01, 150.47, 147.24, 141.30, 137.39, 132.77, 131.95, 129.36, 127.58, 127.09, 124.09, 120.89, 119.76, 40.53, 24.60.

### 6-bromo-4-(2-(6-methylpyridin-2-yl)-5,6-dihydro-4H-pyrrolo[1,2-b]pyrazol-3-yl)quinoline

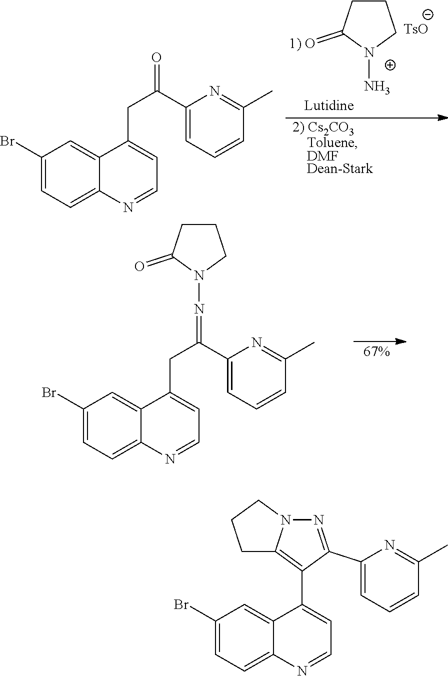

To a stirred solution of 2-(6-bromoquinolin-4-yl)-1-(6-methylpyridin-2-yl)ethan-1-one (2.57 g, 7.53 mmol) in DMF (15.5 mL, 6 volumes), toluene (26 mL, 10 volumes) and 2,6-lutidine (2.6 mL, 1 volume) at rt was added 1-aminopyrrolidin-2-one tosylate salt (2.05 g, 7.53 mmol, 1 equiv.) and stirred at rt for 5 minutes. The reaction was heated to reflux under Dean-Stark conditions until most of the starting material had been consumed by TLC. The reaction mixture was cooled to rt where it was charged with Cs₂CO₃ (4.17 g, 12.8 mmol, 1.7 equiv.) and heated back to reflux. The reaction was monitored for disappearance of the intermediate and once all was consumed, toluene was distilled until the reaction mixture reacted 145° C. and then cooled to rt, split between water (30 mL) and EtOAc (30 mL) and the phases separated. The aqueous phase was extracted with EtOAc (2×20 mL), the combined organic extracts were washed with 5% LiCl aqueous solution (2×20 mL) and the organic layer was dried (MgSO₄), evaporated and the residue was chromatographed (0-10% MeOH in DCM) to give the title compound (2.05 g, 67%) as a brown solid.

¹H NMR (400 MHz, cdcl3) δ 8.85 (d, J=4.4 Hz, 1H), 7.98 (d, J=8.9 Hz, 1H), 7.92 (d, J=2.2 Hz, 1H), 7.71 (dd, J=8.9, 2.2 Hz, 1H), 7.35 (t, J=7.7 Hz, 1H), 7.31 (d, J=4.4 Hz, 1H), 7.11 (d, J=7.8 Hz, 1H), 6.92 (d, J=7.6 Hz, 1H), 4.37 (t, J=7.2 Hz, 2H), 2.90-2.84 (m, 2H), 2.75-2.66 (m, 2H), 2.24 (s, 3H).

¹³C NMR (101 MHz, cdcl3) δ 158.22, 153.60, 153.24, 151.25, 150.22, 147.11, 146.66, 140.88, 136.29, 132.50, 131.34, 128.72, 128.66, 123.03, 121.77, 120.20, 118.86, 48.33, 25.98, 24.19, 23.15.

UPLC-MS: 407 m/z [M+H]

### Compound of Formula I: 4-(2-(6-methylpyridin-2-yl)-5,6-dihydro-4H-pyrrolo[1,2-b]pyrazol-3-yl)quinolin-6-ol

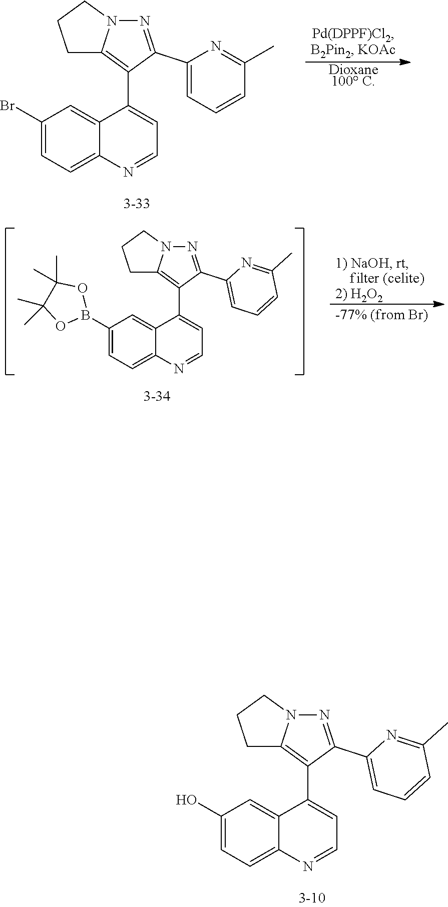

To a flame dried flask under N₂ was added 6-bromo-4-(2-(6-methylpyridin-2-yl)-5,6-dihydro-4H-pyrrolo[1,2-b]pyrazol-3-yl)quinoline (200 mg, 0.493 mmol), Pd(dppf)Cl₂ (36 mgs, 0.050 mmol, 0.1 eq.), KOAc (145 mgs, 1.48 mmol, 3 equiv.) and bis(pinacolato)diboron (137 mgs, 0.542 mmol, 1.1 eq). To the reaction flask was then added degassed dioxane (5 mL) and heated to 100° C. for 2 hours, at which point the reaction was cooled to rt, and diluted with 1 M NaOH (4.93 mL, 4.93 mmol, 10 equiv.) and 30% H₂O₂ (46.6 μL, 0.542 mmol, 1.1 equiv.) added dropwise. After 2 further hours at rt, the reaction was quenched with 1.6 M sodium sulphite (1 mM, 625 μL). After stirring for 30 minutes, the reaction mixture was checked for the presence of peroxides, filtered through a pad of Celite® and taken to pH 8 with 37% HCl. The mixture was diluted with water (5 mL) and extracted with EtOAc (3×5 mL) and chloroform (1×5 mL). The combined organic extracts were dried (MgSO₄), evaporated and the residue was chromatographed (0-100% EtOAc in hexanes) to give title compound 1 (130 mgs, 77%).

¹H NMR (400 MHz, DMSO) δ 9.66 (s, 1H), 8.58 (d, J=4.4 Hz, 1H), 7.86 (d, J=9.0 Hz, 1H), 7.56 (t, J=7.7 Hz, 1H), 7.48 (d, J=7.8 Hz, 1H), 7.29-7.16 (m, 2H), 6.96 (d, J=7.5 Hz, 1H), 6.91 (d, J=2.7 Hz, 1H), 4.27 (t, J=7.2 Hz, 2H), 2.79 (t, J=6.5 Hz, 2H), 2.69-2.55 (m, 2H), 1.89 (s, 3H).

¹³C NMR (101 MHz, DMSO) δ 156.49, 155.08, 152.01, 151.70, 146.55, 146.24, 143.18, 139.37, 136.48, 130.60, 128.71, 122.47, 121.16, 117.73, 109.89, 106.98, 47.86, 25.53, 23.41, 22.50.

IR (film): 3412, 2949, 2833, 1650, 1618, 1508, 1236, 1010 cm⁻¹

1.2. Synthesis of the Compound of Formula I from p-Methoxyaniline—Route (a):

### 6-methoxy-4-methylquinoline

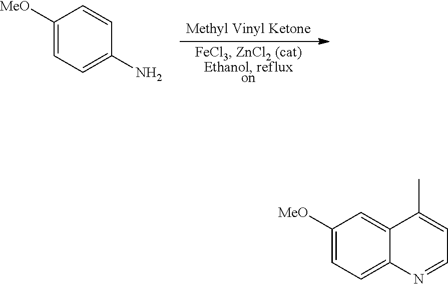

To a stirred solution of p-anisidine (5 g, 40.6 mmol) and ZnCl₂ (719 mg, 5.3 mmol, 0.13 equiv.) in ethanol (20 mL) at rt was added FeCl₃ (17.45 g, 107.6 mmol, 2.65 equiv.) portion wise over 30 minutes. The reaction mixture was stirred for 30 minutes, when methyl vinyl ketone (3.38 mL, 40.6 mmol, 1 equiv.) was added and heated to 75° C. for two hours. It was then cooled to rt and poured into NaOH (1M, 220 mL) at 0° C. The mixture was extracted with EtOAc (3×40 mL) and the combined organic extracts were dried (MgSO₄), evaporated and the residue was chromatographed (0-100% EtOAc in hexanes) to give the title compound (2.88 g, 41%) as a brown oil.

¹H NMR (400 MHz; CDCl₃) δ 8.65 (d, J=4.4 Hz, 1H), 8.02 (d, J=9.2 Hz, 1H), 7.37 (dd, J=9.2, 2.8 Hz, 1H), 7.21 (d, J=4.4 Hz, 1H), 7.19 (d, J=2.8 Hz, 1H), 3.96 (s, 3H), 2.67 (s, 3H).

### 2-(6-methoxyquinolin-4-yl)-1-(6-methylpyridin-2-yl)ethan-1-one

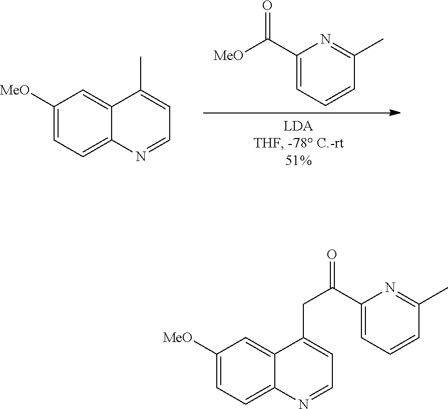 [SMILES: COc1ccc2nccc(C)c2c1 ; COC(=O)c1cccc(C)n1 ; COc1ccc2nccc(CC(=O)c3cccc(C)n3)c2c1]

To a stirred solution of 6-methoxy-4-methylquinoline (2.5 g, 14.4 mmol) in THF (80 mL) at −78° C. was added LDA (33.19 mmol, 2.3 equiv.). The reaction mixture was stirred for 30 minutes when methyl 6-methylpicolinate (3.27 g, 21.6 mmol, 1.5 eq) was added dropwise to the reaction mixture over 30 minutes. The reaction was left at −78° C. and allowed to warm to rt overnight. It was then quenched with sat. NH₄Cl (20 mL), diluted with EtOAc (40 mL) and the phases separated. The aqueous phase was extracted with chloroform (2×50 mL), the combined organic extracts were dried (MgSO₄), evaporated and the residue was chromatographed (0-100% EtOAc in hexanes) to give the title compound (1.91 g, 51%) as a brown oil;

### 6-methoxy-4-(2-(6-methylpyridin-2-yl)-5,6-dihydro-4H-pyrrolo[1,2-b]pyrazol-3-yl)quinoline

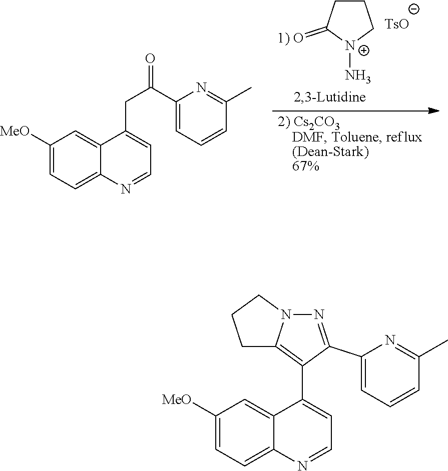

To a stirred solution of 2-(6-methoxyquinolin-4-yl)-1-(6-methylpyridin-2-yl)ethan-1-one (1.60 g, 5.47 mmol) in DMF (9.6 mL, 6 volumes), toluene (16 mL, 10 volumes) and 2,6-lutidine (1.6 mL, 1 volume) at rt was added 1-aminopyrrolidin-2-one tosylate salt (1.49 g, 5.47 mmol, 1 eq) and stirred at rt for 5 minutes. The reaction was heated to reflux under Dean-Stark conditions until most of the starting material had been consumed by TLC. The reaction mixture was cooled to rt where it was charged with Cs₂CO₃ (3.029 g, 9.299 mmol, 1.7 equiv.), left for 5 minutes before being heated back to reflux. The reaction was monitored for disappearance of the intermediate and once all was consumed, the reaction was cooled to rt and split between water (50 mL) and EtOAc (50 mL). The aqueous phase was extracted (2×25 mL), the combined organic extracts were washed with 5% LiCl aqueous solution (2×50 mL) and the organic layer was dried (MgSO₄), evaporated and the residue was chromatographed (0-10% MeOH in DCM). The fractions containing the product were dissolved in a small amount of CHCl₃ and diluted slowly with hexane until the mixture appeared to be slightly cloudy. The flask was left in the fridge for 48 hours were crystals were formed. (1.28 g, 67%) as a white solid;

¹H NMR (400 MHz, cdcl₃) δ 8.74 (d, J=4.4 Hz, 1H), 7.98 (d, J=9.2 Hz, 1H), 7.35-7.20 (m, 4H), 6.96-6.85 (m, 3H), 4.37 (t, J=7.2 Hz, 2H), 3.54 (s, 3H), 2.91 (br s, 2H), 2.75-2.65 (m, 2H), 2.38 (s, 3H).

¹³C NMR (101 MHz, cdcl₃) δ 158.62, 157.67, 153.65, 151.83, 147.66, 146.69, 144.89, 139.90, 136.43, 131.17, 128.25, 122.41, 122.22, 121.93, 119.50, 110.50, 104.00, 55.52, 48.46, 26.19, 24.58, 23.32.

HRMS (ESI): m/z [M+H]+ calculated for C₂₂H₂₀N₄O: 356.1637; found: 356.1639.

### Compound of Formula I: 4-(2-(6-methylpyridin-2-yl)-5,6-dihydro-4H-pyrrolo[1,2-b]pyrazol-3-yl)quinolin-6-ol

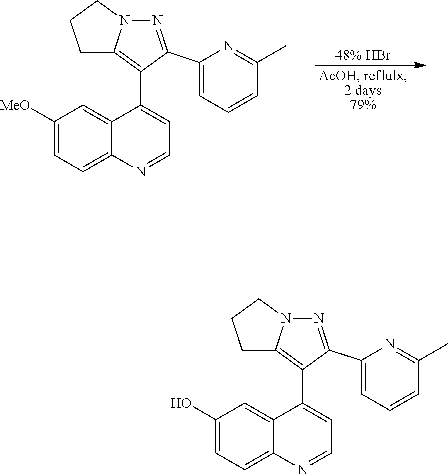 [SMILES: COc1ccc2nccc(-c3c(-c4cccc(C)n4)nn4c3CCC4)c2c1 ; "48% HBr" ; "AcOH, reflulx,2 days79%" ; Cc1cccc(-c2nn3c(c2-c2ccnc4ccc(O)cc24)CCC3)n1]

To a stirred solution of 6-methoxy-4-(2-(6-methylpyridin-2-yl)-5,6-dihydro-4H-pyrrolo[1,2-b]pyrazol-3-yl)quinoline (432 mg, 1.21 mmol) in acetic acid (5 mL) at rt was added HBr (5 mL, 44.45 mmol, 36.7 equiv.). The reaction mixture was stirred for 48 hrs at 120° C. before being cooled to rt and the solvent removed in vacuo. The residue was split between water (5 mL) and EtOAc (10 mL) and taken to pH 8 with 40% NaOH, phases separated, and the aqueous phase extracted with EtOAc (3×10 mL). The combined organic extracts were dried (MgSO₄), evaporated and the residue was suspended in refluxing toluene for one hour before been cooled to rt over an hour. The solid was filtered and dried in vacuo to give the title compound (328 mg, 79%) as an off-white solid:

¹H NMR (400 MHz, DMSO) δ 9.66 (s, 1H), 8.58 (d, J=4.4 Hz, 1H), 7.86 (d, J=9.0 Hz, 1H), 7.56 (t, J=7.7 Hz, 1H), 7.48 (d, J=7.8 Hz, 1H), 7.29-7.16 (m, 2H), 6.96 (d, J=7.5 Hz, 1H), 6.91 (d, J=2.7 Hz, 1H), 4.27 (t, J=7.2 Hz, 2H), 2.79 (t, J=6.5 Hz, 2H), 2.69-2.55 (m, 2H), 1.89 (s, 3H).

¹³C NMR (101 MHz, DMSO) δ 156.49, 155.08, 152.01, 151.70, 146.55, 146.24, 143.18, 139.37, 136.48, 130.60, 128.71, 122.47, 121.16, 117.73, 109.89, 106.98, 47.86, 25.53, 23.41, 22.50.

IR (film): 3412, 2949, 2833, 1650, 1618, 1508, 1236, 1010 cm⁻¹

HRMS (ESI): m/z [M+H]+ calculated for C₂₁H₁₈N₄O: 342.1481; found: 342.1480.

1.3. Synthesis of the Prodrug of Formula III:

### 1-(4-hydroxy-3-methoxyphenyl)-2-((4-(2-(6-methylpyridin-2-yl)-5,6-dihydro-4H-pyrrolo[1,2-b]pyrazol-3-yl)quinolin-6-yl)oxy)ethan-1-one

The compound of formula I, 4-(2-(6-methylpyridin-2-yl)-5,6-dihydro-4H-pyrrolo[1,2-b]pyrazol-3-yl)quinolin-6-ol (42 mg, 0.12 mmol) and Cs₂CO₃ (134 mg, 0.41 mmol) were dissolved in 1.5 mL of anhydrous DMF and heated, under N₂ atm. at 60° C. for 1.5 h. A solution of 4-(2-bromoacetyl)-2-methoxyphenyl pivalate in 0.5 mL of anhydrous DMF was then added via cannula to the bright green suspension. The resulting mixture was stirred at at 60° C. for 2 hours. Then the reaction was cooled to room temperature, diluted with water (15 mL) and quenched with 0.5 mL of a 1 M solution of HCl. The resulting aqueous layer was extracted with EtOAc (3×20 mL), the organic layer was dried over anhydrous MgSO₄ and the solvent was removed in vacuo. The crude was purified via column chromatography (0 to 20% of MeOH in DCM) to afford a brown oil that was treated with LiOH.H₂O (13 mg, 0.31 mmol) in 1.5 mL of MeOH for 1 hour. The reaction was quenched with 1M HCl (0.3 mL), diluted with water and extracted with EtOAc (3×20 mL). The organic layer was dried over anhydrous MgSO₄, the solvent was removed in vacuo and the crude was chromatographed (0 to 20% of MeOH in EtOAc) to yield 51.9 mg (82%) of the title compound.

¹H NMR (400 MHz, CDCl₃) δ 8.73 (d, J=4.6 Hz, 1H), 8.11 (d, J=9.2 Hz, 1H), 7.50 (d, J=1.9 Hz, 1H), 7.47-7.43 (m, 2H), 7.32-7.26 (m, 2H), 7.06-6.95 (m, 3H), 6.89 (d, J=7.6 Hz, 1H), 4.94 (bs, 2H), 4.27 (t, J=7.3 Hz, 2H), 3.93 (s, 3H), 2.82 (bs, 2H), 2.61 (bs, 2H), 2.24 (s, 3H) ppm.

¹³C NMR (101 MHz, CDCl₃) δ 192.3, 158.5, 156.3, 153.4, 152.0, 151.6, 147.5, 147.2, 146.9, 144.2, 141.1, 136.5, 130.7, 128.3, 127.1, 123.5, 122.7, 122.6, 122.0, 119.2, 114.5, 110.2, 110.1, 105.7, 70.4, 56.2, 48.4, 26.0, 24.3, 23.2 ppm.

IR (Film): 3074, 2958, 2925, 1686, 1620, 1589, 1509, 1428, 1274, 1225, 1198, 1088 cm⁻¹.

HRMS (ESI): m/z [M+H]+ calculated for C₃₀H₂₇N₄O₄: 507.2027; found: 507.2016.

Mp: 162-163° C.

1.4. Synthesis of the Prodrug of Formula IV:

### 4-(2-(6-methylpyridin-2-yl)-5,6-dihydro-4H-pyrrolo[1,2-b]pyrazol-3-yl)quinolin-6-yl [1,4′-bipiperidine]-1′-carboxylate

A solution of the compound of formula I synthesized in 1.1. (47 mg, 0.14 mmol) in 3 mL of CHCl₃ was prepared and transferred, under N₂ to a Schlenk flask containing 3 Å activated molecular sieves. The solution was stirred at room temperature for 1 hour. A solution of [1,4′-bipiperidine]-1′-carbonyl chloride (32 mg, 0.14 mmol, 1 eq.), Et₃N (23 μL, 0.17 mmol, 1.2 eq.) and a crystal of 4-dimethylaminopyridine in 1 mL of CHCl₃ was then added dropwise. The resulting mixture was stirred overnight. Some starting material was detected by TLC the following day, so another solution of [1,4′-bipiperidine]-1′-carbonyl chloride (16 mg, 0.07 mmol, 0.5 eq.), Et₃N (12 μL, 0.08 mmol, 0.6 eq.) and a crystal of 4-dimethylaminopyridine in 0.5 mL of CHCl₃ was added to the reaction mixture and it was stirred overnight. The reaction was quenched by adding 20 mL of H₂O. The resulting aqueous layer was extracted with EtOAc (3×20 mL), dried over MgSO₄ and concentrated to afford the crude product which was purified by column chromatography (eluted with a gradient of 10-20% MeOH in DCM) to obtain 64 mg (87%) of the desired product as an orange oil.

¹H NMR (400 MHz, DMSO) δ 8.80 (d, J=4.4 Hz, 1H), 8.01 (d, J=9.1 Hz, 1H), 7.56 (dd, J=24.0, 7.5 Hz, 2H), 7.47 (dd, J=9.1, 2.6 Hz, 1H), 7.38 (d, J=4.4 Hz, 1H), 7.28 (d, J=2.6 Hz, 1H), 6.95 (d, J=7.8 Hz, 1H), 4.27 (t, J=7.2 Hz, 2H), 4.01 (dd, J=37.1, 14.3 Hz, 2H), 3.65-3.14 (m, 1H), 2.81 (d, J=16.6 Hz, 4H), 2.61 (m, 3H), 1.82 (s, 3H), 1.74 (d, J=10.6 Hz, 3H), 1.44 (m, 10H).

¹³C NMR (101 MHz, DMSO) δ 156.59, 152.55, 151.92, 151.57, 149.18, 148.60, 147.08, 145.62, 141.10, 136.57, 130.20, 127.54, 124.70, 122.63, 121.25, 117.67, 116.92, 109.27, 61.31, 49.59, 47.95, 25.53, 23.29, 22.41.

UPLC-MS: 537.8 (M+H)

1.5. Synthesis of the Hydrochloride Salt of the Prodrug of Formula IV:

### 1-(1-(((4-(2-(6-methylpyridin-2-yl)-5,6-dihydro-4H-pyrrolo[1,2-b]pyrazol-3-yl)quinolin-6-yl)oxy)carbonyl)piperidin-4-yl)piperidin-1-ium chloride

Compound of formula IV (65 mg, 0.12 mmol) was dissolved in DCM (1.5 mL) and treated with 0.3 mL (1.21 mmol) of a 2M solution of HCl in dioxane during 2h. Then the solvent was removed to afford a pale orange solid (70 mg, 100%).

¹H NMR (400 MHz, Methanol-d4) δ 9.18 (d, J=5.6 Hz, 1H), 8.35 (d, J=9.2 Hz, 1H), 8.12 (d, J=5.6 Hz, 1H), 8.06 (t, J=7.9 Hz, 1H), 8.02-7.93 (m, 2H), 7.74 (d, J=7.9 Hz, 1H), 7.28 (d, J=7.9 Hz, 1H), 4.56-4.39 (m, 3H), 4.31-4.28 (m, 1H), 3.59-3.56 (m, 2H), 3.52-3.45 (m, 2H), 3.19-2.95 (m, 6H), 2.88 (s, 3H), 2.85-2.81 (m, 1H), 2.25-2.20 (m, 2H), 2.01-1.85 (m, 7H), 1.60-1.50 (m, 1H) ppm.

¹³C NMR (101 MHz, Methanol-d4) δ 157.0, 153.3, 153.2, 152.2, 151.0, 147.2, 146.1, 145.5, 144.9, 137.62, 131.4, 129.3, 128.0, 124.6, 124.5, 124.1, 119.5, 111.0, 66.9, 64.6, 51.4, 50.4, 44.3, 44.0, 27.5, 27.2, 27.1, 24.5, 24.2, 22.9, 20.1, 15.5 ppm.

IR (Film): 3411, 3048, 2933, 2857, 1716, 1589, 1420, 1195, 1020, 804 cm⁻¹.

HRMS (ESI): m/z [M+H]+ calculated for C₃₂H₃₇N₆O₂: 537.2973; found: 537.2968.

Mp: 214-215° C.

### Example 2: In Vitro TGFβ Inhibitor Activity

2.1 Molecular Affinity and Selectivity Assays

The affinity, measured as Kd (dissociation constants), of the compound of formula I for the ALK family was determined by using the KINOMEscan™ Technology from Eurofins. Selectivity in front of ALK1 (ACVRL1), ALK2 (ACVR1), ALK3 (BMPR1A), ALK4 (ACVR1B), ALK6 (BMPR1B), ACVR2B and TGFβR2 was determined.

The KINOMEscan screening platform employs an active-site directed competition binding assay to quantitatively measure interactions between test compounds and kinases.

KINOMEscan assays do not require ATP and thereby report true thermodynamic interaction affinities.

The assay is performed by combining three components: DNA-tagged kinase; immobilized ligand; and a test compound. The ability of the test compound to compete with the immobilized ligand is measured via quantitative PCR of the DNA tag.

Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kds were determined using an 11-point 3-fold compound dilution series with three DMSO control points being the top concentration 30 μM. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

Compounds that bind the kinase active site and directly (sterically) or indirectly (allosterically) prevent kinase binding to the immobilized ligand, will reduce the amount of kinase captured on the solid support. Conversely, test molecules that do not bind the kinase have no effect on the amount of kinase captured on the solid support. Dissociation constants (Kds) for test compound-kinase interactions are calculated by measuring the amount of kinase captured on the solid support as a function of the test compound concentration.

As seen in Table 1 below, the compound of formula I is 8.6 times more potent in ALK5 affinity than galunisertib. Comparison has been made taking into account galunisertib published data (Jonathan M et al., Oncotarget, 2018, 9 (6):6659-6677) using the same technology.

TABLE 1
KINOMEscan ™ affinity data

| | | | Selectivity | | Selectivity |
|---|---|---|---|---|---|
| | IC₅₀ (nM) | | against ALK5 | | improve- |
| TARGET | (I) | Galunisertib | (I) | Galunisertib | ment |
| ALK1 | 11000 | 24900 | 550 | 145 | 3.8 |
| (ACVRL1) | | | | | |
| ALK2 | 7500 | 35700 | 375 | 208 | 1.8 |
| (ACVR1) | | | | | |
| ALK3 | 6900 | 16800 | 345 | 98 | 3.5 |
| (BMPR1A) | | | | | |
| ALK4 | 13 | 77.7 | 0.7 | 0.5 | 1.4 |
| (ACVR1B) | | | | | |
| ALK5 | 20 | 172 | 1 | 1 | |
| (TGFBR1) | | | | | |
| ALK6 | 160 | 471 | 8.0 | 2.7 | 2.9 |
| (BMPR1B) | | | | | |
| ACVR2B | 100 | 694 | 5.0 | 4.0 | 1.2 |
| TGFBR2 | 2000 | 208 | 100 | 1.2 | 83 |

Enzymatic activity inhibition, measured as IC₅₀, of the compound of formula I, the prodrugs of formula II and IV, of the invention, and prior art compounds 337, 338 and galunisertib, for ALK5 was, also determined by using the Kinase Profiler service from Eurofins.

Briefly, human ALK5 was incubated with 8 mM MOPS pH 7.0, 0.2 mM EDTA, 1 mM MnCl2, 2 mg/mL casein, 10 mM MgAcetate and [γ-33P-ATP] (specific activity approx. 500 cpm/pmol, concentration as required). The reaction was initiated by the addition of the MgATP mix. After incubation for 40 minutes at room temperature, the reaction was stopped by the addition of a 3% phosphoric acid solution. 10 μL of the reaction was then spotted onto a P30 filtermat and washed three times for 5 minutes in 75 mM phosphoric acid and once in methanol prior to drying and scintillation counting.

Compounds were tested using a 9-point curve with half-Log serial dilutions. The top concentration tested was 10 μM. Test compounds were prepared in 100% DMSO.

Results shown in Table 2, show that the compound of formula I is 2.5 times more potent than Galunisertib. The results for the prodrugs of formula III and IV, of the invention, and prior art compounds 337, 338 are also shown in the same table below:

TABLE 2
KinaseProfiler ™ enzymatic activity data
Enzyme activity inhibition
IC₅₀ (nM)

| Compound | ALK5 (TGFBR1) |
|---|---|
| Compound formula (I) | 177 |
| Galunisertib | 448 |
| Prodrug of formula (III) | 836 |
| Prodrug of formula (IV) | 2284 |
| Reference compound 337 | 320 |
| Reference compound 338 | 371 |

As seen in table 2, the results show that, unexpectedly, the compound of formula (I) is 2.1 times more potent than reference compound 338, when structurally said compounds only differ in the position of the phenolic hydroxyl. Additionally, similar results are shown in comparison to reference compound 337, being the compound of formula (I) 1.8 times more potent than said compound, knowing that structurally said compounds only differ in the position of the phenolic hydroxyl and in the absence or presence of a methyl group in the pyridine moiety.

As seen, the prior art identified a family of compounds which provide activity as inhibitors of TGFβ pathway, however, surprisingly, present invention shows that the type of substitutions and position in the core structure thereof, is key in providing the unexpected improved potency shown by the compound of formula (I).

Moreover, Li et al. (J. Med. Chem. 2008, 51(7): 2302-2306) disclose that electron-deficient and relatively small groups were tolerated at the 6-position, teaching away from the introduction of electron-donating groups such as the hydroxyl featured in the 6-position by the compound of formula (I).

In terms of the prodrugs, compound of formula I is 4.7 and 12.9 times more potent than the prodrugs of the compound of formula (I) having formula (III) and (IV), respectively, highlighting that the TGFβ inhibitory activity is blocked in vitro.

Using scanEDGE (KINOMEscan, Eurofins) assays, the selectivity of the compound of formula I against a panel of 97 proteins was also measured. The selectivity profile of the compound of formula was good.

In addition, a safety assessment was performed by using the SafetyScreen44™ service from Eurofins. The targets therein are all strongly associated with in vivo adverse drug reactions as referenced in the publication Reducing safety-related drug attrition: the use of in vitro pharmacological profiling (Bowes J et al., Nature Review Drug Discovery 2012, 11(12): 909-922). The panel represents a rational first step in preliminary safety assessments as they provide early identification of potential off-target interactions for the optimization of safety margins. This Safety Panel includes both enzymatic and binding assays. The percentage of inhibition for the compound of formula I at 10 μM was lower than 50% for all proteins tested. Taking into account the compound's binding affinity for ALK5 (20 nM), selectivity ratios for the proteins in the SafetyScreen44 panel are greater than 500-fold.

2.2. In Vitro Luciferase Assay

HEK293T cells were purchased from the ATCC and cultured in DMEM supplemented with L-glutamine and 10% fetal bovine serum (Life Technologies) at 37° C. and 5% CO₂. The cells were seeded in 24-well plates and transfected with plasmids encoding 12×CAGA-Firefly_Luc and Tk-Renilla_Luc (75 and 10 ng per well, respectively), using polyethylenimine (Polysciences) as transfection reagent. After 7h, medium was replaced for starvation medium (DMEM+0.05% FBS). The next day, cells were treated with galunisertib, compound of formula I, and prodrugs compound of formula III and compound of formula IV, from a 10 mM stock solution in DMSO, diluted between 103 and 10⁸ times (as indicated), as well as 5 ng/ml recombinant human TGFB1 (Peprotech). Luciferase activity was measured 16h later using the Dual Luciferase Assay kit (Promega): media were aspirated, and cells were lysed in 200 μl passive lysis buffer (kit) for 20 minutes. Bioluminescence was measured in a Berthold Lumat LB6507 luminometer (18 μl reagents, 10 seconds measurements).

When TGβ binds to the receptor, phosphorylation of the TGFBR1 occurs which in turn phosphorylates SMAD2/3, which then forms a complex with SMAD4 and enters the nucleus. The SMAD2/3-SMAD4 complex then binds to a promotor (or TGF-β response element) and with various co-factors, allows the transcription of the downstream gene, or luciferase reporter.

When adding the luciferase substrate (luciferin) to the cell lysates, emission of light implies an active TGF-β pathway due to the production of pSMAD2/3-SMAD4, whereas no or little observed light would imply inhibition of the pathway by the inhibitor. Finally, the results could be normalized by the addition of Stop & Glo® which quenches the firefly luciferase enzyme/substrate and activates the Renilla transfection control, producing light which was again measured by the luminometer and used to normalize the data.

FIG. 1 shows the how the TGFβ signaling inhibitory capacity of the compound of formula I is more than 2 times that of galunisertib, i.e. galunisertib IC₅₀ value found was 359 nM and that the compound of formula I 165 nM, whereas prodrugs of compound of formula I, compound of formula III and compound of formula IV have up to 8 times lower TGFβ signaling inhibitory capacity, compared to compound of formula I. This means that the TGFβ signaling inhibitory capacity is strong in the compound of formula I, due to its specific structural features and, additionally, the modifications made to obtain the prodrugs, compound of formula III and compound of formula IV, successfully cage or block the TGFβ signaling inhibitory activity.

### Example 3: TGFβ Inhibitor In Vivo Studies

Mouse tumor organoids (MTOs) are derived from compound transgenic mice (conditional mutations in Apc, Kras, Trp53 and Tgfbr2, as well as bearing Lgr5-EGFP/CreER^{T2}) of the C57BL6/J strain and cultured as described in Tauriello et al. 2018 Nature 554(7693): 538-543. doi: 10.1038/nature25492. Organoids are tumor epithelial cells grown in a 3D matrix where they adopt an organotypic architecture, typically as spheroids with a lumen. In this way, cells retain a relatively normal polarity (which means that the outside surface, inside/luminal surface and cell-cell contacts are individually specialized), which is more physiological than in conventional 2D cell culture. Moreover, these organoids are capable of recreating a complex tumor architecture that recapitulates human cancers, including the recruitment of a rich and pro-tumorigenic (i.e. TGFβ rich) stroma; a system to transplant human-like tumors, and study liver metastasis, not previously described.

For in vivo anti-cancer efficacy and TGFβ inhibitor studies, C57BL/6J mice were purchased from Janvier at 6 weeks of age and injected at 7-8 weeks. MTOs were trypsinized (digesting the matrix and disaggregating organoids into single cells), counted and 300,000 cells were injected in HBSS (Hank's Balanced Salt Solution, Lonza) into the spleen, thus delivering the cells directly into the portal vein that connects the intestine to the liver. In all experiments described in present invention MTO129 were used (Tauriello et al. Nature. 2018).

Treatment with compounds (compound of formula I and galunisertib) occurred by gavage (tube feeding) with a suspension of between 4 and 120 mg/ml in a 1% sodium carboxymethylcellulose (Sigma), 0.4% sodium dodecyl sulfate (Sigma), 0.085% polyvinylpyrrolidone, 0.05% antifoam-A (Sigma) in milli-Q water. Mice were treated twice a day with 150 μl for 14 days or until endpoint.

In mice where treatment started two weeks after MTO transplantation and lasted three days before sacrifice, the effect of treatment on biomarker was assessed. Tissue sections of 4 μm were stained by immunohistochemistry with antibodies specific for phosphorylated SMAD2 (Cell Signaling, 3108), employing procedures well known in the art. FIG. 2 shows cellular nuclei positive for phospho-SMAD2 in control treated animals (vehicle), marked with arrows, but not in mice treated with galunisertib or with compound of formula I or the prodrug thereof, the compound of formula IV.

FIG. 3 shows liver tumor count (LiMs) of compound of formula I and of galunisertib at different doses (molar equivalents of 0.3×, 1×, 3× and 9× the commonly mouse dose of 80 mg/kg b.i.d. of galunisertib, which translates to 2 mg per mouse when assuming an average weight of 25 g). In mice, especially in mouse metastatic colorectal cancer, galunisertib is not effective at the standard 1× dose of 160 mg/Kg/day; in the abovementioned MTO paper (Tauriello et al. Nature et al. 2018), we needed to use doses up to 1600 mg/kg/day (10×) to effectively treat metastatic cancers. However, said doses can result in toxicity (cardiac toxicity, muscle tissue and cartilage defects, bone development and altered inflammatory responses in skin and gut). In patients, treatment doses were reduced to 80 to 150 mg b.i.d in a regime of 14 days on-14 days off. As shown in FIG. 3 the compound of formula I resulted in a significant lower amount of liver tumors, when compared to galunisertib.

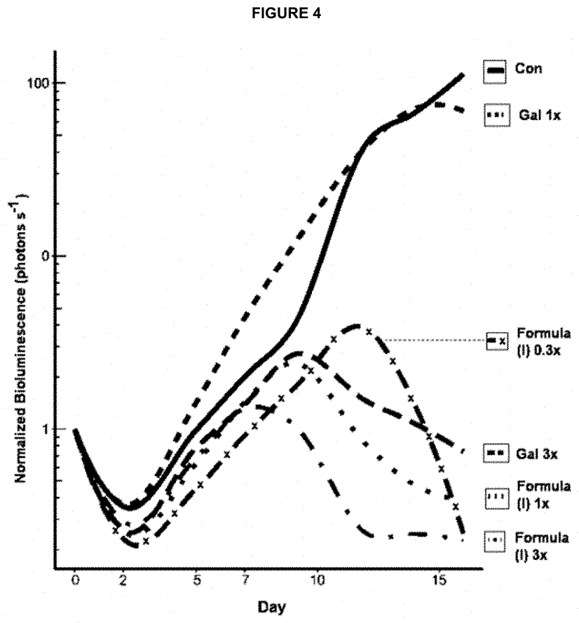

MTOs expressing a luciferase reporter were measured by bioluminescence imaging, using retroorbital injection of 50 μl 15 mg/ml D-luciferin potassium salt (Resem BV). Photons per second from the region spanning thorax/abdomen were quantified and normalized to the measure at the day of injection. FIG. 4 shows the metastatic tumor growth inhibitory capacity of the compound of formula I and galunisertib at different doses (0.3×, 1×, 3× and 9× the mouse dose of 80 mg/kg b.i.d, i.e. 160 mg/kg/day) during 12 days of treatment (day 3 until day 14). As can be seen in FIG. 4, the 1× dose (mouse dose of 160 mg/Kg/day) was not effective for galunisertib, and had to be increased to a 3× dose (corresponding to 480 mg/Kg/day) to be mostly effective (the 9× dose of 1440 mg/kg/day was highly effective, as seen in FIG. 3), whereas the compound of formula I was effective at a molar equivalent of 0.3× dose (corresponding to 42.5 mg/Kg/day; this is after correction for the molecular weight ratio of formula I to galunisertib monohydrate of 0.88:1, as the molecular weight of the compound of formula I is 342.4 and that of galunisertib 387.4) showing a surprisingly and strongly improved antitumor activity in vivo.

### Example 4: Toxicity Assessments

For the safety assessment regarding proteins often linked to toxicity, refer to the SafetyScreen44 assay in example 2.1.

To assess the toxicity of the compound of formula I in vitro, HEK293T cells were seeded in 96-well plates and treated for 24h with indicated compounds. DMSO was used as a control.

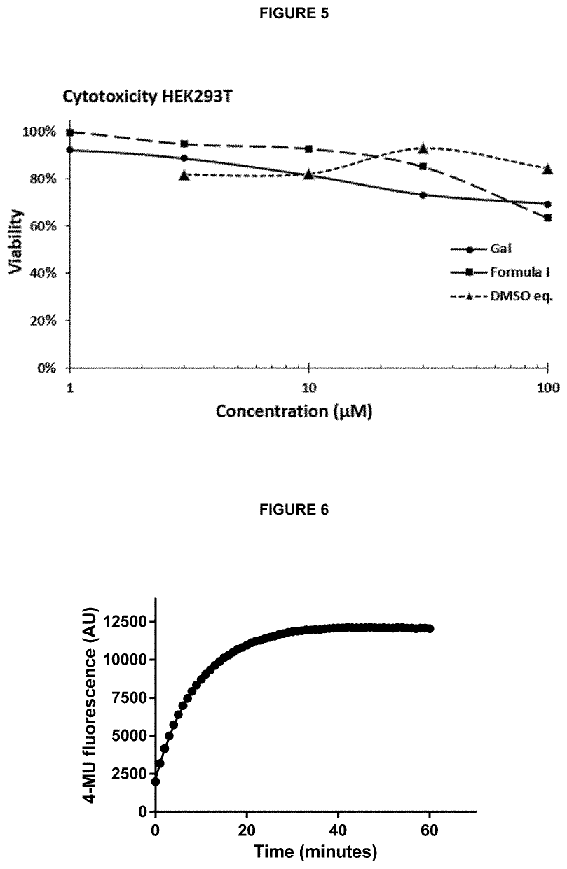

The compound of formula I or galunisertib was added at different concentrations, ranging from 1 and 100 μM. Cell viability was assessed by the XTT assay (Biological Industries), according to the manufacturer's instructions, incubating for 4h prior to measurement. Cell viability was 64% at 100 μM and 93% at 30 μM (FIG. 5) which indicates a very low toxicity at physiological levels, especially when considering that the IC₅₀ value of inhibition of ALK5 of compound of formula I is in the nM range.

For the assessment of in vivo toxicity, mice were inoculated with MTO129 and treated with different molar equivalent doses of galunisertib and compound of formula I during two weeks as described in FIG. 3. Number of cured mice, i.e. mice with no metastases at experimental end points, were score. Intestines, skin samples, hearts, thoracic skeletons and limbs of these mice were fixed in 10% formalin, phosphate buffered (Sigma). Soft tissues were embedded in paraffin blocks, sectioned and stained with haematoxylin & eosin. Bones were first decalcified for 2 weeks before embedding, sectioning and staining. An experienced pathologist analyzed tissues for histopathological abnormalities. The macroscopically obvious pectus excavatum phenotype (caved in sternum) was found to associate to defects in the sternocostal cartilage areas (cartilage present between sternum bones and ribs). Furthermore, the cartilage ossification zone at the ends of the long bones was analysed for defects in the hypertrophic zone. No pathological anomalies were observed in an analysis of the skin and intestine with 1× doses of either galunisertib or the compound of formula I.

TABLE 3
Efficacy and toxicity comparison galunisertib (Gal) vs the compound
of formula I. Toxicity per number of mice tested is scored, combined
and taken as percentage;

| | Control | Gal (LY) | | | (I) | | | |
|---|---|---|---|---|---|---|---|---|
| Side effect | 0x | 1x | 3x | 9x | 0.3x | 1x | 3x | 9x |
| Sternum | 2/12 | 0/5 | 1/4 | 4/7 | 0/5 | 0/5 | 1/5 | 1/4 |
| Long bones | 0/8 | n.d. | 0/5 | 2/3 | n.d. | n.d. | 1/5 | 3/4 |
| Combined | 2/20 | 0/5 | 1/9 | 6/10 | 0/5 | 0/5 | 2/10 | 4/8 |
| Toxicity (%) | 10% | 0% | 11% | 60% | 0% | 0% | 20% | 50% |
| Efficacy | x | 0% | ~70% | ~100% | ~90% | ~90% | 100% | 100% |
| (Cure rate) | | | | | | | | |

n.d: not determined

Galunisertib was effective (>50% efficacy) from a 3× dose equivalent (480 mg/kg/day), and was toxic (toxicity >50%) at 9× (1440 mg/kg/day). The compound of formula I was effective at a 0.3× dose equivalent (42.5 mg/kg/day), and was also toxic (toxicity >50%) at 9× dose equivalent (1272 mg/kg/day).

Based on this, the therapeutic index (ratio between toxic dose/effective dose) of the compound of formula I compared to galunisertib may be calculated.

Galunisertib therapeutic index=9/3=3. Compound of formula I therapeutic index=9/0.3=30. The results indicate that the compound of formula I provides at least a 10-fold improvement versus galunisertib.

### Example 5: Synthesis of the Prodrug of Formula VII

To couple the acetovanillone-fragment to 4-hydroxytamoxifen, first a halogenated acetovanillone fragment was synthesized departing from commercial acetovanillone. 1-(4-(benzyloxy)-3-methoxyphenyl)ethan-1-one

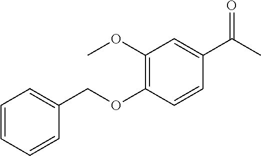 [SMILES: COc1cc(C(C)=O)ccc1OCc1ccccc1]

To a stirred solution of acetovanillone (6 g, 36.1 mmol) in DMF (120 mL) at rt was added K2CO3 (7.48 g, 54.15 mmol, 1.5 equiv.) followed by benzyl bromide dropwise (4.66 mL, 38.99 mmol, 1.08 equiv.). The reaction mixture was stirred for 14 hrs at 40° C. before being cooled down to rt where it was poured into 1 L of water/ice mixture and stirred for 10 minutes. The suspension was then filtered using a Büchner funnel under vacuum, the solid rinsed with water (3×70 mL) at 0° C., before being left under vacuum in the Büchner funnel for one hour. The solid was then transferred into a round bottom flask and dried to a constant weight on the high vacuum line giving 8.99 g (97% yield) of the titled compound. (C. Miesch, T. Emrick, J. Colloid Interface Sci. 2014, 425, 152-8).

¹H NMR (400 MHz, Chloroform-d) δ 7.55 (d, J=2.0 Hz, 1H), 7.50 (dd, J=8.4, 2.0 Hz, 1H), 7.43 (dd, J=8.2, 1.4 Hz, 2H), 7.38 (m, 2H), 7.34-7.29 (m, 1H), 6.91-6.87 (m, 1H), 5.23 (s, 2H), 3.94 (s, 3H), 2.55 (s, 3H).

### 1-(4-(benzyloxy)-3-methoxyphenyl)-2-bromoethan-1-one (5-8a)

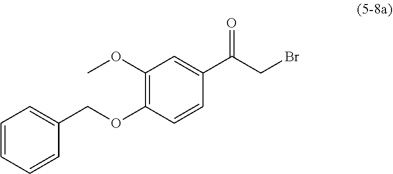 [SMILES: COc1cc(C(=O)CBr)ccc1OCc1ccccc1]

To a stirred solution of the previously obtained 1-(4-(benzyloxy)-3-methoxyphenyl)ethan-1-one (7.86 g, 30.69 mmol) in EtOH (150 mL) at 60° C. was added bromine (4.90 g, 30.69 mmol, 1 equiv.). The reaction mixture was stirred for 6 h, before being left to cool slowly to room temperature overnight. In the morning the reaction mixture was cool to 0° C. where it was left stirring for an hour. It was then filtered through a glass Buchner funnel, washed with cold EtOH (2×30 mL), and then left to dry on the high vacuum line. This afforded the title compound (7.25 g, 71%) as a white solid pure by ¹H NMR.

¹H NMR (400 MHz, cdcl3) δ 7.58-7.49 (m, 2H), 7.46-7.29 (m, 5H), 6.92 (d, J=8.4 Hz, 1H), 5.25 (s, 2H), 4.39 (s, 2H), 3.95 (s, 3H).

The synthesis of the prodrug of formula VII departing from 4,4′-(2-phenylbut-1-ene-1,1-diyl)diphenol (CAS number 4120-45-0) may be illustrated as follows in Scheme VIII:

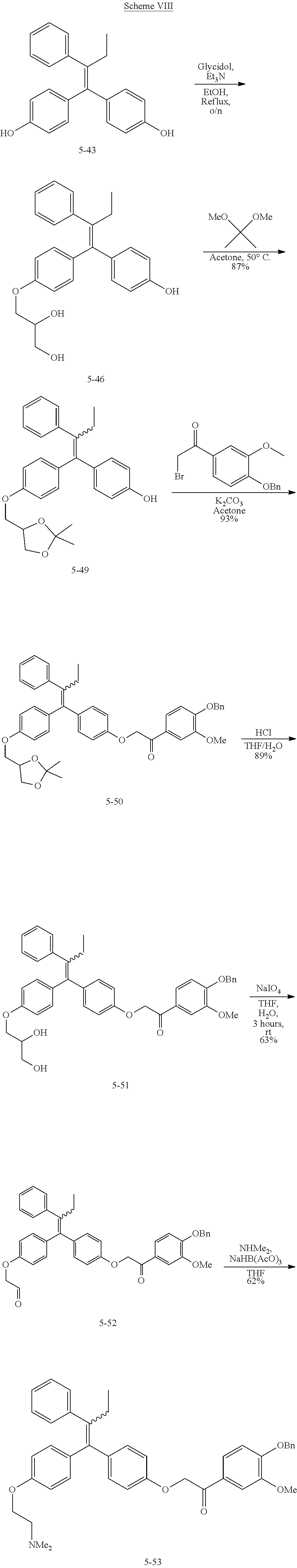

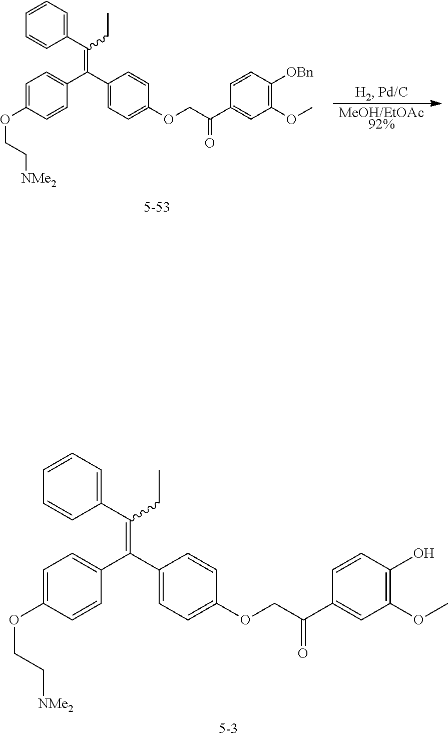 [SMILES: CCC(=C(c1ccc(OCCN(C)C)cc1)c1ccc(OCC(=O)c2ccc(OCc3ccccc3)c(OC)c2)cc1)c1ccccc1 ; "H2, Pd/C" ; "MeOH/EtOAc92%" ; CCC(=C(c1ccc(OCCN(C)C)cc1)c1ccc(OCC(=O)c2ccc(O)c(OC)c2)cc1)c1ccccc1 ; "5-3"]

### 3-(4-(1-(4-hydroxyphenyl)-2-phenylbut-1-en-1-yl)phenoxy)propane-1,2-diol, (5-46)

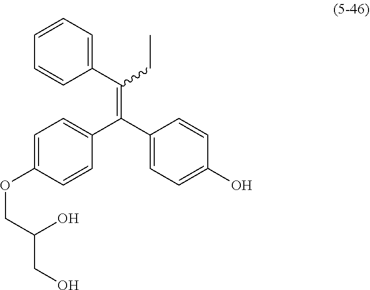 [SMILES: CCC(=C(c1ccc(O)cc1)c1ccc(OCC(O)CO)cc1)c1ccccc1]

To a solution of diol 5-43 (1.6 g, 5.06 mmol) was added NEt₃ (0.706 mL, 5.06 mmol, 1 eq.) and glycidol (0.301 mL, 4.55 mmol, 0.9 eq.) in EtOH (24 mL) in a sealed flask and heated up to 80 degrees overnight. In the morning, the reaction mixture was cooled to room temperature and the solvent was removed in vacuo. The residue split between EtOAc (40 mL) and saturated ammonium chloride (40 mL), before the organic layer was dried over MgSO₄ and concentrated to dryness in vacuo. The residue was purified by flash column chromatography giving the titled compound as a thick colorless oil (735 mg, 37.2% yield).

¹H NMR (400 MHz, cd₃od) δ 7.19-7.06 (m, 1H), 7.04 (d, J=8.4 Hz, 1H), 6.96 (t, J=5.7 Hz, 1H), 6.82-6.74 (m, 1H), 6.67 (d, J=8.5 Hz, 1H), 6.62-6.55 (m, 1H), 6.42 (d, J=8.5 Hz, 1H), 4.13-3.80 (m, 1H), 3.79-3.53 (m, 1H), 2.49 (p, J=7.5 Hz, 1H), 0.92 (t, J=7.4 Hz, 1H).

¹³C NMR (101 MHz, cd₃od) δ 159.14, 158.29, 157.25, 156.36, 144.11, 144.10, 141.99, 141.80, 139.74, 139.71, 137.87, 137.50, 136.34, 135.98, 133.02, 133.00, 131.57, 130.89, 130.87, 128.84, 126.94, 115.84, 115.18, 115.09, 114.35, 71.84, 71.76, 70.34, 70.08, 64.21, 64.14, 29.89, 29.85, 13.94. ** due to the fact that the double bond is a 1:1 mixture of cis and trans isomers, and also the fact that the glycidol used was racemic, there are more carbons than expected. This is due to diastereomers.**

### 4-(1-(4-((2,2-dimethyl-1,3-dioxolan-4-yl)methoxy)phenyl)-2-phenylbut-1-en-1-yl)phenol, (5-49)

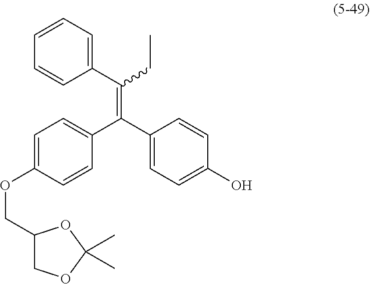 [SMILES: CCC(=C(c1ccc(O)cc1)c1ccc(OCC2COC(C)(C)O2)cc1)c1ccccc1]

To a solution of 5-46 (106 mg, 0.271 mmol) was added to a flask under N₂ and dissolved in acetone (3 mL) at room temperature where p-TSA (7.7 mg, 0.040 mmol, 0.15 eq.) and 2,2-dimethoxypropane (66 μL, 542 mmol, 2 eq.) was added at 50 degrees and left to react for 5 hours. Once all the starting material had disappeared by TLC, the reaction mixture was cooled to rt where it was diluted with 10% NaHCO₃ (2 mL) and EtOAc (6 mL), the phases were separated, and the aqueous layer was extracted with EtOAc (2×6 mL), the organic layers were combined and dried over MgSO₄, before being concentrated in vacuo. The residue was then purified through silica giving the titled compound (102 mgs, 87% yield) as a colourless oil.

¹H NMR (400 MHz, cdcl3) δ 7.19-7.04 (m, 7H), 6.92-6.83 (m, 1H), 6.82-6.69 (m, 3H), 6.56-6.52 (m, 1H), 6.48-6.44 (m, 1H), 4.96 (s, 0.5H), 4.70 (s, 0.5H), 4.54-3.77 (m, 5H), 2.52-2.42 (m, 2H), 1.49-1.34 (m, 6H), 0.92 (t, J=7.4 Hz, 3H).

¹³C NMR (101 MHz, cdcl₃) δ 157.20, 156.37, 154.22, 153.41, 142.53, 141.20, 141.16, 137.63, 137.61, 136.69, 136.33, 136.23, 135.89, 132.09, 131.92, 130.76, 130.59, 129.67, 127.83, 127.81, 125.92, 114.93, 114.23, 114.04, 113.29, 109.79, 109.69, 74.03, 73.98, 68.71, 68.42, 66.89, 66.80, 29.01, 29.00, 26.79, 26.73, 25.37, 25.34, 13.59.

** due to the fact that the double bond is a 1:1 mixture of cis and trans isomers, and also the fact that the glycidol used was racemic, there are more carbons than expected. This is due to diastereomers.

IR (film): 3400, 2920, 2851, 1722, 1673, 1598, 1496, 1273 cm⁻¹

HRMS (ESI): m/z (M+H) calculated for C₂₈H₃₀O₄: 430.2144; found: 430.2143.

### 1-(4-(benzyloxy)-3-methoxyphenyl)-2-(4-(1-(4-((2,2-dimethyl-1,3-dioxolan-4-yl)methoxy)phenyl)-2-phenylbut-1-en-1-yl)phenoxy)ethan-1-one, (5-50)

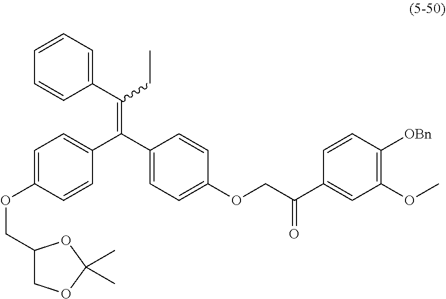 [SMILES: CCC(=C(c1ccc(OCC(=O)c2ccc(OCc3ccccc3)c(OC)c2)cc1)c1ccc(OCC2COC(C)(C)O2)cc1)c1ccccc1]

To a stirred solution of 5-49 (500 mg, 1.16 mmol, 1 eq.) in acetone (15 mL) was added K₂CO₃ (321 mg, 2.32 mmol, 2 eq.) added followed by alpha bromo ketone 5-8a (582 mg, 1.74 mmol, 1.5 eq.) where it was left to stir overnight at room temperature. The following morning, the solvent was removed in vacuo, where it was then diluted with EtOAc (50 mL) and water (30 mL). The phases were separated, and the aqueous layer was extracted with EtOAc (2×20 mL), the organic layers were combined and dried over MgSO₄, before being concentrated in vacuo. The residue was then purified through silica giving the titled compound (737 mg, 92.7% yield) as a colorless oil.

¹H NMR (400 MHz, cdcl₃) δ 7.61-7.27 (m, 7H), 7.19-6.84 (m, 10H), 6.78-6.71 (m, 2H), 6.59-6.50 (m, 2H), 5.30-5.05 (m, 4H), 4.53-4.34 (m, 1H), 4.22-4.02 (m, 1H), 3.99-3.86 (m, 5jH), 3.85-3.74 (m, 1H), 2.46 (q, J=7.5 Hz, 2H), 1.49-1.34 (m, 6H), 0.91 (td, J=7.3, 1.0 Hz, 3H).

¹³C NMR (101 MHz, cdcl₃) δ 193.25, 193.20, 157.39, 156.98, 156.55, 156.15, 153.18, 153.05, 149.91, 149.82, 142.62, 142.58, 141.53, 141.48, 137.66, 137.23, 136.76, 136.73, 136.26, 136.23, 132.10, 132.07, 130.78, 130.75, 129.80, 128.86, 128.84, 128.32, 128.30, 128.13, 128.12, 127.98, 127.31, 127.30, 126.09, 122.75, 122.68, 114.53, 114.20, 113.79, 113.45, 112.38, 112.32, 110.94, 110.91, 109.88, 109.78, 74.17, 74.12, 70.98, 70.95, 70.91, 70.78, 68.89, 68.60, 67.06, 66.97, 56.27, 56.23, 29.19, 29.14, 26.98, 26.89, 25.52, 25.50, 13.74, 13.73.

** due to the fact that the double bond is a 1:1 mixture of cis and trans isomers, and also the fact that the glycidol used was racemic, there are more carbons than expected. This is due to diastereomers.**

UPLC-MS: 685.9 (M+H)

### 1-(4-(benzyloxy)-3-methoxyphenyl)-2-(4-(1-(4-(2,3-dihydroxypropoxy)phenyl)-2-phenylbut-1-en-1-yl)phenoxy)ethan-1-one, (5-51)

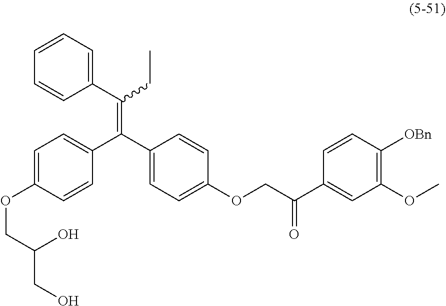 [SMILES: CCC(=C(c1ccc(OCC(=O)c2ccc(OCc3ccccc3)c(OC)c2)cc1)c1ccc(OCC(O)CO)cc1)c1ccccc1]

To a stirred solution of 5-50 above (634 mg, 1.09 mmol) was dissolved in 6:1 THF:water (20 mL) where 2M HCl (2 mL) was added at room temperature. The reaction was followed by TLC and upon consumption of all the SM, was quenched with 10% NaHCO₃ (10 mL). The reaction mixture was diluted with EtOAc and the phases were separated, the aqueous phase was extracted with EtOAc (5 mL) and chloroform (5 mL). The combined organic extracts were dried over MgSO₄, before being concentrated in vacuo. The residue was then purified through silica (MeOH/DCM) giving the titled compound (527 mg, 89% yield) as a colorless oil.

¹H NMR (400 MHz, cdcl₃) δ 7.61-7.30 (m, 7H), 7.19-7.05 (m, 7H), 6.95-6.85 (m, 3H), 6.79-6.72 (m, 2H), 6.59-6.51 (m, 2H), 5.26-5.02 (m, 4H), 4.23-3.53 (m, 8H), 2.50-2.40 (m, 2H), 0.91 (td, J=7.3, 1.1 Hz, 3H).

### 2-(4-(1-(4-(2-(4-(benzyloxy)-3-methoxyphenyl)-2-oxoethoxy)phenyl)-2-phenylbut-1-en-1-yl)phenoxy)acetaldehyde, (5-52)

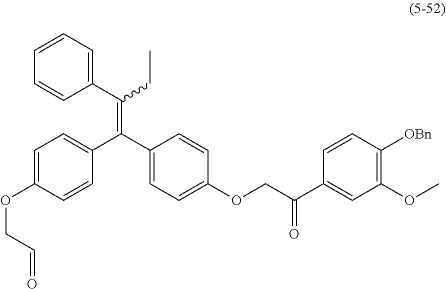 [SMILES: CCC(=C(c1ccc(OCC=O)cc1)c1ccc(OCC(=O)c2ccc(OCc3ccccc3)c(OC)c2)cc1)c1ccccc1]

To a stirred solution of 5-51 above (250 mg, 0.388 mmol) in DCM (7 mL) was added NalO₄ (249 mgs, 1.16 mmol, 3 eq.) in water (2 mL) at room temperature. The reaction was stirred vigorously for 3 hours where most of the starting material had been consumed. It was diluted with brine (4 mL) before the phases were separated. The organic phase was dried (MgSO₄), and the solvent was removed in vacuo, before being purified by flash column chromatography giving the titled compound as a colorless oil (155 mgs, 63% yield).

¹H NMR (400 MHz, cdcl₃) δ 9.88 (br s, 0.5H), 9.77 (br s, 0.5H), 7.62-7.28 (m, 7H), 7.20-7.05 (m, 7H), 6.94-6.84 (m, 3H), 6.81-6.70 (m, 2H), 6.60-6.49 (m, 2H), 5.33-4.98 (m, 4H), 3.93 (d, J=14.5 Hz, 4H), 2.51-2.38 (m, 2H), 0.92 (dt, J=8.5, 4.2 Hz, 3H).

### 1-(4-(benzyloxy)-3-methoxyphenyl)-2-(4-(1-(4-(2-(dimethylamino)ethoxy)phenyl)-2-phenylbut-1-en-1-yl)phenoxy)ethan-1-one (5-54)

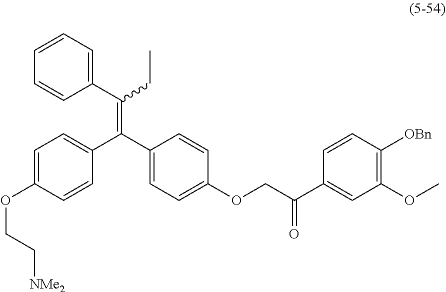 [SMILES: CCC(=C(c1ccc(OCCN(C)C)cc1)c1ccc(OCC(=O)c2ccc(OCc3ccccc3)c(OC)c2)cc1)c1ccccc1]

To a stirred solution of 5-52 (143 mg, 0.233 mmol, 1 eq.) in THF (5 mL) under N₂ was added a 2M solution of dimethylamine in THF (0.175 mL, 0.350 mmol, 1.5 eq.) at room temperature, followed by Na(AcO)₃BH (75 mg, 0.700 mmol, 3 eq.) portion wise. The reaction was left to react for 5 hours, at which point no SM was visible by TLC. The reaction mixture was then diluted with brine (3 mL) and EtOAc (10 mL) and the phases were separated after stirring for 15 minutes. The aqueous phase was extracted with EtOAc (2×10 mL) and chloroform (1×10 mL), the organic extracts were combined, dried (MgSO₄) and the solvent removed in vacuo. The residue was purified by flash column chromatography (DCM/MeOH—0-15%) giving the titled compound as a thick oil (93 mgs, 62% yield)

¹H NMR (400 MHz, cdcl₃) δ 7.62-7.55 (m, 1H), 7.55-7.48 (m, 1H), 7.46-7.28 (m, 5H), 7.21-7.06 (m, 7H), 6.94-6.81 (m, 3H), 6.78-6.71 (m, 2H), 6.59-6.45 (m, 2H), 5.24 (s, 1H), 5.22 (s, 2H), 5.06 (s, 1H), 4.26 (t, J=5.1 Hz, 1H), 4.11-4.07 (m, 1H), 3.95 (s, 1.5H), 3.91 (s, 1.5H), 3.11 (dt, J=36.9, 4.3 Hz, 2H), 2.63 (s, 3H), 2.57 (s, 3H), 2.46 (q, J=6.7 Hz, 2H), 0.91 (td, J=7.4, 1.8 Hz, 3H).

### 2-(4-(1-(4-(2-(dimethylamino)ethoxy)phenyl)-2-phenylbut-1-en-1-yl)phenoxy)-1-(4-hydroxy-3-methoxyphenyl)ethan-1-one, (Prodrug of Formula VII)

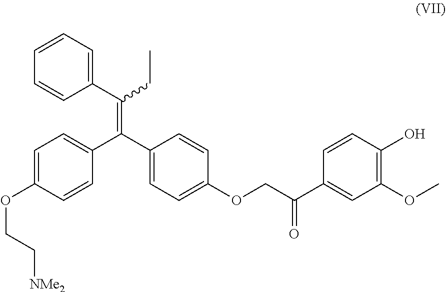 [SMILES: CCC(=C(c1ccc(OCCN(C)C)cc1)c1ccc(OCC(=O)c2ccc(O)c(OC)c2)cc1)c1ccccc1]

To a stirred solution of 5-54 (94 mg, 0.146 mmol) in degassed 10% MeOH/EtOAc (12 mL) was added Pd/C (9 mg) at room temperature. The N₂ atmosphere was replaced with a H₂ atmosphere in 3 cycles with a balloon of H₂ and vacuum. The reaction was followed by TLC and as the SM had been consumed, the atmosphere was replaced with N₂ and left to purge for 5 minutes. The reaction mixture was filtered through celite and the solvent removed in vacuo. The residue was purified by flash column chromatography (DCM/MeOH—gradient) giving the titled compound as a thick oil (74 mg, 92% yield)

¹H NMR (400 MHz, cdcl₃) δ 7.65-7.58 (m, 1H), 7.55-7.50 (m, 1H), 7.17-7.05 (m, 7H), 7.00-6.86 (m, 3H), 6.79-6.71 (m, 2H), 6.59-6.51 (m, 2H), 5.23 (s, 1H), 5.07 (s, 1H), 4.14 (t, J=5.6 Hz, 1H), 4.01-3.93 (m, 4H), 2.86 (t, J=5.6 Hz, 1H), 2.77 (t, J=5.4 Hz, 1H), 2.50-2.40 (m, 5H), 2.38 (s, 3H), 0.91 (td, J=7.4, 2.0 Hz, 3H).

### Example 6: LigF Enzymatic Assays

6.1 Synthesis of 4-Methylumbelliferone Acetovanillone

4-methylumbelliferone (4-MU) derivatives are known compounds. The synthesis of MUAV can be illustrated as follows:

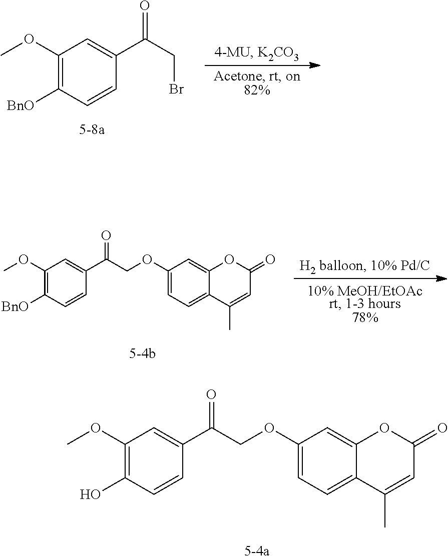

### 7-(2-(4-(benzyloxy)-3-methoxyphenyl)-2-oxoethoxy)-4-methyl-2H-chromen-2-one, (5-4b)

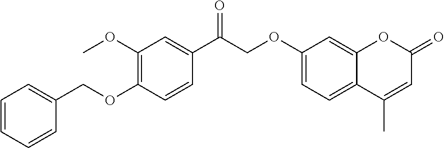 [SMILES: COc1cc(C(=O)COc2ccc3c(C)cc(=O)oc3c2)ccc1OCc1ccccc1]

To a stirred solution of 4-methylumbelliferone I(400 mg, 2.27 mmol) in acetone (30 mL) at 25° C. was added 1-(4-(benzyloxy)-3-methoxyphenyl)-2-bromoethan-1-one (5-8a synthesized in Example 5) (967 mg, 2.72 mmol, 1.2 equiv.). The reaction mixture was stirred for 16 h, before being filtered and the solid residue washed with acetone (2×30 mL). The solvent was then removed under reduced pressure, before being suspended in toluene and heated until a solution was formed. The flask was then left to cool to RT over 90 minutes, where it was filtered, washed with cold toluene (10 mL), and left to dry on the high vacuum line. This afforded the titled compound as a white solid (801 mg, 82%).

¹H NMR (400 MHz, cdcl3) δ 7.57-7.30 (m, 8H), 6.94 (d, J=4.5 Hz, 2H), 6.78 (d, J=2.5 Hz, 1H), 6.14 (d, J=1.2 Hz, 1H), 5.31 (s, 2H), 5.26 (s, 2H), 3.95 (s, 3H).

### 7-(2-(4-hydroxy-3-methoxyphenyl)-2-oxoethoxy)-4-methyl-2H-chromen-2-one, (5-4a)

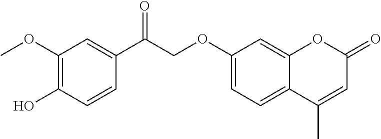 [SMILES: COc1cc(C(=O)COc2ccc3c(C)cc(=O)oc3c2)ccc1O]

To a pressure reactor was added 5-4b (500 mg, 1.16 mmol) and 10% Pd/C (25 mg, 5% wrt to mass of sm) in degassed 10% MeOH/EtOAc (10 mL) at rt. The reactor was charged with 1 bar(g) of hydrogen and the reaction followed by TLC by taking samples using an appropriate needle which is able to withstand pressure. After 4 hours, the starting material had been consumed. The hydrogen was released from the reactor and replaced with nitrogen via 3 vacuum-nitrogen cycles. The reaction mixture was diluted with CHCl₃ (20 mL) and filtered through Celite. The filtrate was concentrated under reduced pressure. The crude was chromatographed (0-10% MeOH:DCM) to give title compound 5-4a (307 mg, 78%) as a white solid.

¹H NMR (400 MHz, Chloroform-d) δ 7.59-7.55 (m, 2H), 7.51 (d, J=8.8 Hz, 1H), 7.00 (d, J=8.1 Hz, 1H), 6.94 (dd, J=8.8, 2.6 Hz, 1H), 6.79 (d, J=2.6 Hz, 1H), 6.18 (br s, 1H), 6.14 (d, J=1.2 Hz, 1H), 5.33 (s, 2H), 3.97 (s, 3H), 2.39 (d, J=1.2 Hz, 3H).

6.2 In Vitro Enzymatic Fluorescence Assays

To study the kinetics of the cleavage of the ether bond in of the prodrugs of formula II and of formula V in general, including the compound of formula III, prodrug of the compound of formula I, the compound of formula VII, prodrug of 4-hydroxytamoxifen, and the compound of formula IX, prodrug of SN-38, and to know the selectivity of the enzyme for the recognition fragment, a fluorescent compound fluorescent 4-methylumbelliferone was used as model compound.

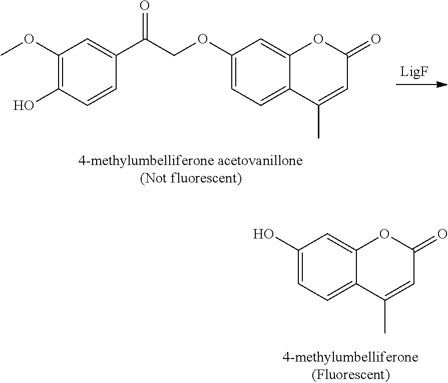 [SMILES: COc1cc(C(=O)COc2ccc3c(C)cc(=O)oc3c2)ccc1O ; "LigF" ; Cc1cc(=O)oc2cc(O)ccc12]

To test the enzymatic activity of LigF, pure LigF protein was expressed by the Protein Expression Core Facility within the IRB Barcelona by inserting the His-tagged LigF DNA into a pOPINF vector and expressed in E. coli BL21 (DE3) Rosetta IysS cells. After expression and lysis, the protein was purified by using a nickel column followed by gel filtration. Noteworthy, the His tags were not removed from the protein after purification.

For in vitro fluorescence assays, purified LigF was diluted to 15 μg/ml in 20 mM Tris-HCl (pH 7.5), 1 mM glutathione and 30 μM 4-methylumbelliferone acetovanillone (MUAV) model compound was added.

Reactions were performed in a 96-well solid polystyrene plate (Corning) and measured with a top-well plate reader (BioTek FL600 fluorometer) at 360 excitation, 485 emission and sensitivity 100.

Due to a rapid rate of production of 4-methylumbelliferone (4-MU) owing to the cleavage of the beta-ether bond of compound MUAV, the protocol described by Masai (E. Masai et al., J. Bacteriol. 2003, 185, 1768-75) was modified by lowering the LigF enzyme concentration. With this optimized protocol, compound MUAV was incubated at room temperature in the presence of a buffer, glutathione (GSH) and LigF, and followed the cleavage by observing the production of 4-MU by fluorescence. Appropriate controls including wells containing no GSH, no LigF, and no MUAV indicated that the ether bond wasn't decomposing under the assay conditions and enzymatic activity was required. Enzyme LigF cleaved product MUAV in approximately 30-40 min (FIG. 6).

For assays in cell culture, cells were stably transduced to express LigF by lentiviral infection with pLenti PGK puro vector and packaging plasmids pCAG-RTR2, pCAG-VSVG and pCAG-KGP1R. After selection with 1 μg/ml puromycin (InvivoGen), infected cells were seeded into 24-well plates, and 4-methylumbelliferone acetovanillone (MUAV) was added. At time points after addition of various concentrations of MUAV, namely 10, 30, and 100 μM, culture medium was taken for measurements of 4-MU fluorescence.

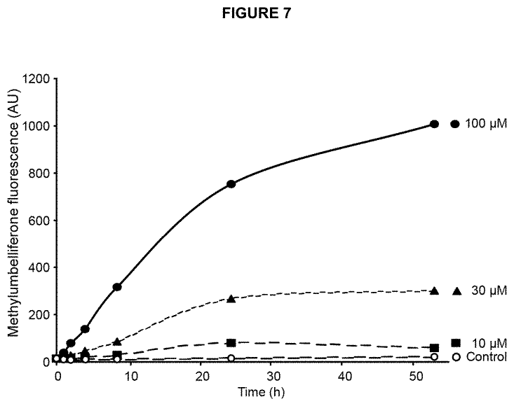

FIG. 7 shows the ether bond of MUAV was indeed cleaved by the LigF expressed by the cells, indicated by a dose-dependent increase in fluorescence. No cleavage was observed in the control experiment: HEK293T cells expressing an empty vector (and not LigF), treated with 100 μM. These experiments indicated that LigF has activity inside mammalian cells and that there is no other enzyme capable of cleaving the beta ether bond, at least in the cells tested.

6.3. In Vitro LigF Assays of the Prodrug of Formula VII

For further test in vitro enzymatic assays with LigF and an acetovanillone-derived prodrug, the compound of formula VII—a prodrug of 4-hydroxytamoxifen—was used.

Similar to the in vitro assay in 5.2., the compound of formula VII was diluted from a 30 mM stock solution in DMSO to 50 μM in 20 mM Tris-HCl (pH 7.5), 1 mM glutathione and 15 μg/ml LigF was added. However, the reaction mixture was incubated at 37° C. and the read-out was different: using HPLC-MS/MS. Reaction samples, as well as a control sample without enzyme and Z-4-OHT (4-hydroxytamoxifen) as a standard were diluted 2:3 in MeOH and 2 μl was injected onto a BioBasic C18 column (5 μm, 2.1×150 mm, Thermo; using a Thermo EC Finnigan Mod Micro AS autosamples and Thermo EC Quarternary pump, Finnigan Mod. Surveyor MS chromatograph) with a water-MeOH gradient (60-90% MeOH in 30 min; 90-100% in 5 min; 100 μl/min flow rate). LC-MS coupling was performed with the Advion Triversa Nanomate (Advion BioSciences, Ithaca, N.Y., USA) as the nanoESI source performing nanoelectrospray through chip technology. The Nanomate was attached to an LTQ-FT Ultra mass spectrometer and operated at a spray voltage of 1.7 kV and a delivery pressure of 0.5 psi in positive mode. In a LTQ-FT Ultra (Thermo Scientif) mass spectrometer, analyses were performed using 40V capillary voltage and 120V tube lens voltage. MS/MS was operated in a data-dependent acquisition (DDA) mode and in selected reaction monitoring (SRM) mode. Survey MS scans were acquired in the FT with the resolution (defined at 400 m/z) set to 100,000. Up to six of the most intense ions per scan were fragmented and detected in the linear ion trap. The ion count target value was 1,000,000 for the survey scan and 50,000 for the MS/MS scan. Target ions already selected for MS/MS were dynamically excluded for 30 s. For SRM, maximum injective was set to 400 ms and 2 microscans average.

Data are represented as XIC (extracted ion chromatogram) peak areas of parallel reaction monitoring (PRM) transitions, using QuanBrowser (Xcalibur software 2.0SR2).

LigF was able to cleave the beta ether bond of the prodrug of formula VII that links the recognition fragment (acetovanillone) and the 4-OHT core, producing 4-OHT. Note that the molecule remained intact when all the other components were present bar the LigF in the control experiments, indicating prolonged stability at body temperature. Unexpectedly the E-isomer of the prodrug of formula VII cleaved at a faster rate than the Z-isomer, resulting in the specific liberation of the isomer (Z)-4-OHT. (The fact that the E-isomer of the prodrug of formula VII cleaves to give (Z)-4-OHT is due to the Cahn-Ingold-Prelog priority rules.)

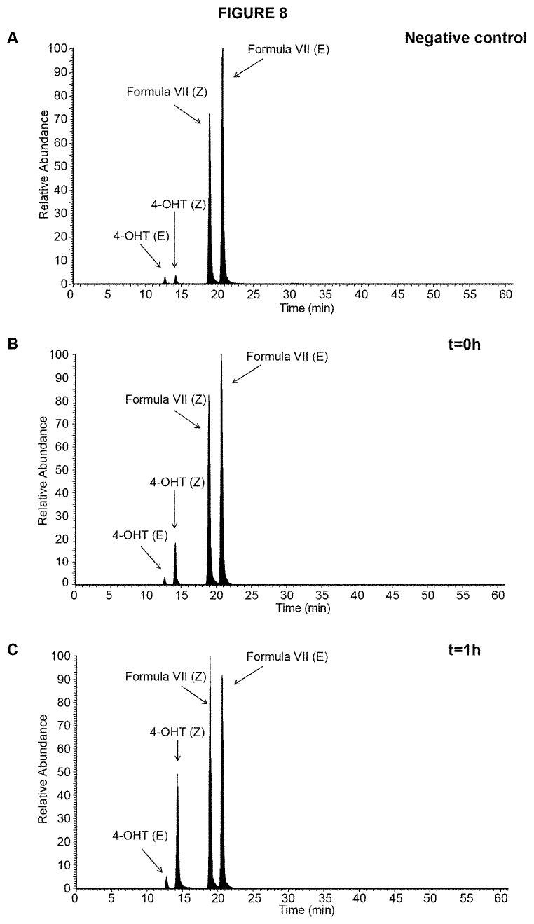

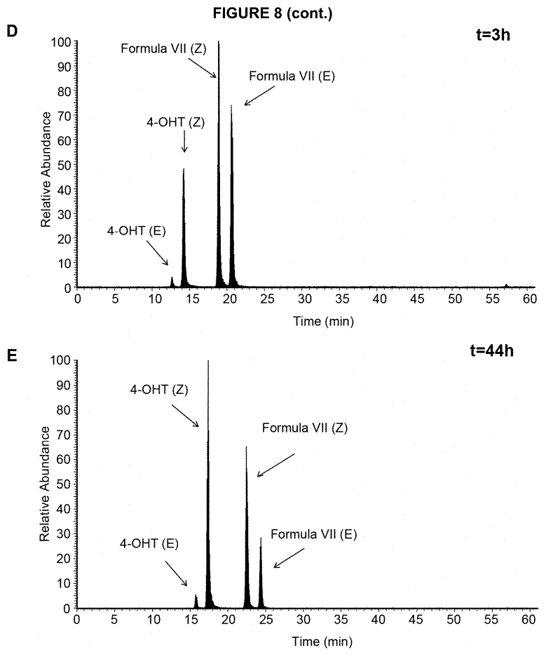

FIG. 8 shows HPLC-MS/MS experiment of the cleavage of the beta ether bond of prodrug of formula VII, by LigF, giving 4-OHT. (A) negative control. (B) Time=0. (C) Time=1 hour. (D) Time=3 hours. (E) Time=44 hours.

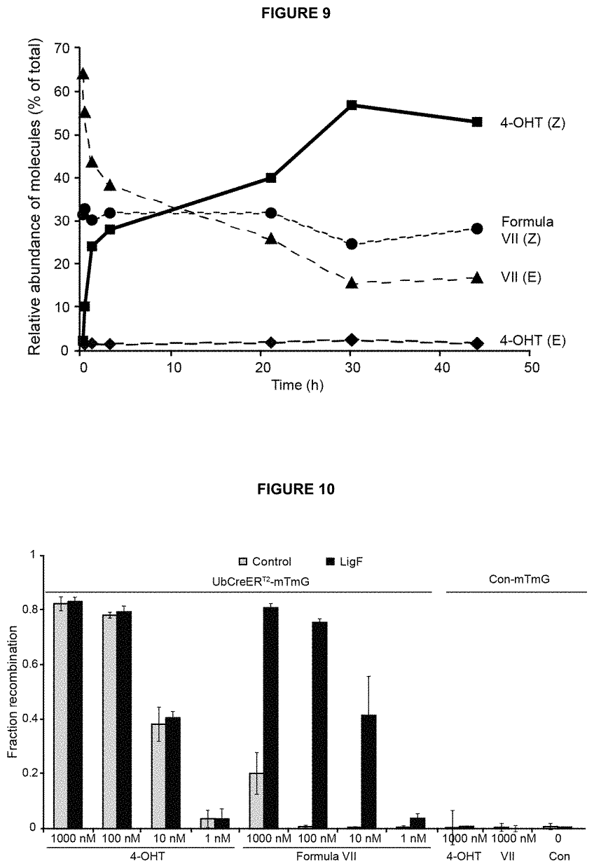

The cleavage of the prodrug of formula VII reached a plateau of 50% after approximately 30 hours and arrived half way to the plateau after 3 hours, which we envisaged to be a sufficient rate to be a useful tool in vivo as seen in FIG. 9.

6.4 Cellular Co-Culture Assays:

These assays were conducted to assess whether this specific LigF enzymatic reaction on the compound of formula VII can occur in a cellular context, including that this prodrug can pass through the cell membrane and that, after cleavage by LigF, 4-OHT can diffuse to neighboring cells and activate mutated ER^{T2} fusion proteins, achieving nuclear activity of the fusion protein.

In this example, those fusion proteins are a Cre-ER^{T2} and an active portion of a transcription factor (NTCF4-ER^{T2})-.

For the first experiment, wherein gene recombination in cells neighboring LigF-expressing cells is demonstrated, mouse embryonic fibroblasts (MEF) were used. The mouse model carries recombination reporter mTmG (JAX stock: 007676). This system expresses the membrane bound tdTomato (tandem dimer tomato; mT stands for membrane Tomato), which is a red fluorescent protein. Upon recombination with an activated Cre enzyme, the coding sequence for mT as well as a stop codon, being between loxP sites, is excised. This results in the expression of membrane-bound enhanced green fluorescent protein (EGFP; mG) instead. Effectively this means that in the presence of a Cre enzyme, or in the presence of a 4-OHT activated Cre-ER^{T2} fusion protein, the cells stop their production of tdTomato and proceed to express the EGFP; this genetic switch can be efficiently quantified by flow cytometry analysis. The mouse strain expressing mTmG was then crossed to a mouse carrying a Cre-ER^{T2} fusion protein in all cells (UbC-Cre-ER^{T2}; Jax Stock: 008085). From the dual transgenic mouse, as well as from the reporter strain without Cre-ER^{T2}, MEFs were harvested from 14-day-old embryos. Briefly, pregnant females were sacrificed 14 days after plug formation, uteri were removed, and embryos were dissected: heads and fetal livers were discarded. The rest was minced and digested with trypsin and grown in 10-cm plates with aforementioned supplemented DMEM.

MEF recombination: MTO129, transduced with pLenti PGK-LigF (puro) or with an equivalent empty vector was trypsinized and single cells added onto UbC-Cre-ER^{T2}; mTmG (or control mTmG-only) MEFs grown in 12-well plates. The next day, either Z-4-OHT or the prodrug of formula VII was added in concentrations ranging from 1000 to 1 nM. Two days later, cells were trypsinized and analyzed by flow cytometry for TdTomato/EGFP fluorescence (Gallios, Beckman Coulter). Fluorescence was analyzed on viable cells in 2 dimensions and scored as percentages in the 4 quadrants. EGFP positive quadrants (Q2 and Q4, with or without TdTomato) were considered recombined and the fraction of the total was calculated. Data are the average (+SD) from two independent experiments.

FIG. 10 shows the results of the assay, wherein bars under UVCre-mTmG show the results obtained with MEFs expressing Cre-ER^{T2}, together with the recombination reporter cassette (mTmG), whereas the columns under Con-mTmG show results with MEFs lacking Cre-ER^{T2} expression, used as a control.

As seen in FIG. 10 there was dose-dependent gene recombination by addition of 4-OHT, regardless of Cre-ER^{T2} expression. When the prodrug of formula VII was added, this achieved a pattern of gene recombination in the MEFs that is very similar to 4-OHT only in MEFs expressing the LigF etherase. In MEFs lacking UbC-ER^{T2}, there was only some recombination at the highest dose, which might be explained by a slight impurity, or by an inherently reduced activity. This indicates that prodrug of formula VII is mostly inactive, but that it was cleaved in MTO129 that expresses Lig-F, co-cultured with the MEFs, and that this reaction was sufficient to allow the liberated 4-OHT to diffuse to the MEFs and effect gene recombination.

For the second experiment, wherein the ER^{T2} fusion product was a transcription factor, LS174T-NTCF4-ER^{T2} (LS-NE) cells (as described in Whissell et al., 2014, Nature Cell Biology, 16, 695-707. Original LS174T cells were purchased from the ATCC) were used in co-culture with Lig-F expressing HEK293T cells. These LS-NE cells were cultured in DMEM supplemented with L-glutamine and 10% fetal bovine serum (Life Technologies) at 37° C. and 5% CO₂.

NTCF4 stands for the N-terminus of transcription factor TCF4. This construct has a dominant negative function, switching off signaling in the Wnt pathway (Wnt-OFF), which is important in development and (intestinal) stem cell regulation. In addition, most colorectal cancer (CRC) cells are addicted to Wnt signaling, such that its inhibition would result in strong responses in gene expression of both pro-tumorigenic stem cell genes (going down, repressed) and cellular differentiation genes (going up, activated). Fusion to ER^{T2} keeps this construct out of the nucleus, and thus inactive. In the CRC cell line LS174T, modified with NTCF4-ER^{T2}, addition of 4-OHT results in the described changes in endogenous gene expression

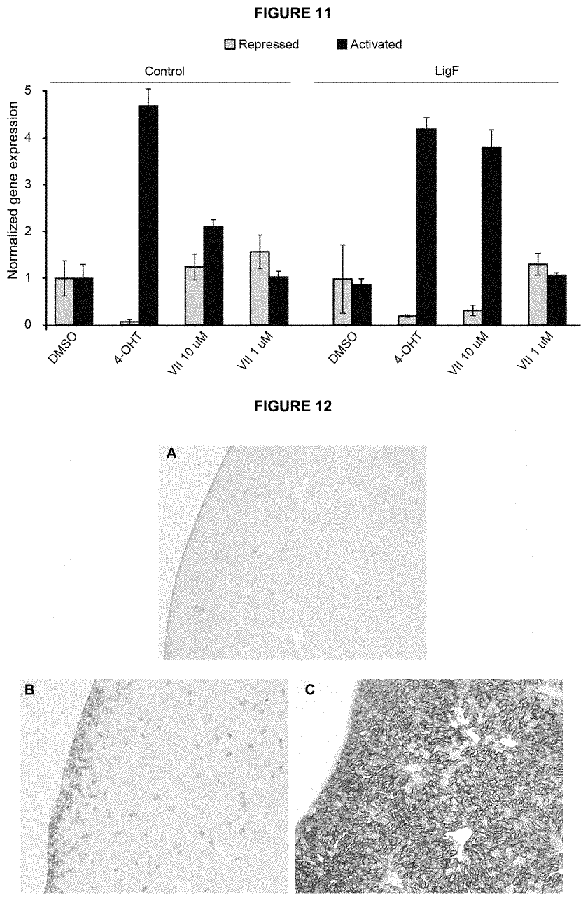

HEK293T cells transduced with pLenti PGK-LigF (puro) or with an equivalent empty vector were co-plated with LS174T-NTCF4-ER^{T2} (LS-NE) cells, in a ratio of 1/10 in 6-well plates. Co-cultures were treated with DMSO, 1 μM Z-4-OHT or the prodrug of formula VII at 1 or 10 μM. After 16h, cells were lysed in trizol for RNA extraction (PureLink RNA Mini Kit, Life Technologies) and cDNA preparation (High Capacity cDNA RT kit, Applied Biosysytems). Stem cell gene ASCL2 (repressed by 4-OHT-mediated Wnt-OFF; Hs_00270888_S1), differentiation gene KRT20 (activated by 4-OHT-mediated Wnt-OFF; Hs_00300643_m1) and housekeeping gene PPIA (Hs_99999904_m1) were analyzed by RT-qPCR using Taqman probes (Applied Biosystems) on a StepOnePlus Real-Time PCR system (Applied Biosystems). Data in FIG. 11 were normalized to the housekeeping gene, and to DMSO controls. Data are the average of technical replicates (+SD) from a single experiment. Whereas in the presence of HEK293T negative for LigF, only 4-OHT was able to induce the transcriptional switch (activation gene KRT20 up and repression gene ASCL2 down), in the presence of HEK293T-LigF, the prodrug of formula VII achieved the same response at 10 μM.

### Example 7: 4-OHT Prodrug of Formula VII In Vivo Studies

Using the UbCre-ER^{T2}; mTmG mice described in example 6, we treated these animals with the prodrug of formula VII at 1 or 5 μmol (approximately 0.6 or 3 mg, respectively) or with a vehicle control (oil). Five days after treatment, mice were sacrificed, and the liver was fixed overnight in 10% phosphate-buffered formalin (sigma) and embedded in paraffin, similar to the description of example 3 (FIG. 1). Sections were cut and stained for EGFP using immunohistochemistry, The results, shown in FIG. 12, indicate that enzymes present in the mouse liver (but not in our cultured cells, see example 6) are able to cleave the prodrug of formula VII, resulting in a dose-dependent activation of gene recombination and EGFP expression.

### Example 8: Additional Studies of TGFβ Signaling Inhibition In Vivo Using Cancer Models

Additional in vivo studies were carried out to further evaluate the efficacy of the compound of formula I of the invention, and the prodrug thereof of formula IV, versus reference compounds Galunisertib and reference compound 338 of the prior art. Mouse tumor organoids (MTO129) were developed as indicated in example 3. For in vivo anti-cancer efficacy and TGFβ signaling inhibition studies, C57BL/6J mice were purchased from Janvier at 6 weeks of age and injected at 7-8 weeks. For transplantation of MTO129 into the liver, cells were trypsinized (digesting the matrix and disaggregating organoids into single cells), counted and 300,000 cells were injected in HBSS (Hank's Balanced Salt Solution, Lonza) into the spleen, thus delivering the cells directly into the portal vein that connects the intestine to the liver.

Compounds (compound of formula I, compound of formula IV, Galunisertib or compound 338) were prepared as suspensions in a vehicle solution composed of 1% sodium carboxymethylcellulose (Sigma), 0.4% sodium dodecyl sulfate (Sigma), 0.085% polyvinylpyrrolidone, 0.05% antifoam-A (Sigma) in milli-Q water. Treatment with compounds occurred by gavage (tube feeding), twice a day with 150 μl of the compound suspension. Control mice were treated with 150 μl of vehicle.

In the experiment shown in FIG. 13, potency of the indicated compounds to inhibit metastasis formation was assessed. Mice were treatments with indicated compounds started two days after MTO129 transplantation and continued during two weeks. Mice were sacrificed at 4 weeks post transplantation and liver metastases (LiMs) were counted by eye on fixed livers. FIG. 3 shows liver tumor count (LiMs) of compound of formula I and of galunisertib at different molar equivalent doses (9×, 3×, 1×, 0.3×) as described in the figure legend. The compound of formula I at 1× dose (70.8 mg/Kg b.i.d) provided a significant improved activity in preventing metastasis formation when compared to reference compound 338, the closest compound structurally to the compound of formula I, at the same molar dose (70.8 mg/Kg b.i.d).

Furthermore, FIG. 13 shows that the compound of formula I is still effective in preventing liver metastasis at a dose of 0.3× (21.25 mg/Kg b.i.d), and this therapeutic effect is comparable to a galunisertib dose of 3×. Additionally, the activity of the compound of formula I was significantly superior to that of reference compound 338, being this highly unexpected since reference compound 338 is a constitutional isomer of the compound of formula I.

The prodrug of the compound of formula I, i.e. formula IV, reduces the number of liver metastases in this assay more robustly than galunisertib.

Additionally, experiments shown in FIG. 14 and FIG. 15 assess the therapeutic efficacy on overt metastatic disease of compound of formula I compared to prior art compound galunisertib, both at 1× equimolar doses (compound of formula I at 70.8 mg/kg b.i.d and galunisertib at 80 mg/Kg b.i.d). For this purpose, the protocol of mouse inoculation with MTO129 described for FIG. 13 was used, but here treatment with compounds started at day 16 post-inoculation. At this time point mice had developed large metastases. Treatments finished at day 24. The results (FIG. 14) show that compound of formula I provides improved efficacy at reducing the number of liver metastasis in this experimental setting when compared to galunisertib at equimolar doses. On the other hand, FIG. 15 shows survival of mice with overt metastatic disease after treatment with compounds has finalized. In these experiments, the vast majority (>90%) of control mice die of liver metastatic disease. Mice treated with the compound of formula I had a higher survival rates than mice treated with galunisertib. The experiments shown in FIG. 13, FIG. 14 and FIG. 15 illustrate the unexpected enhanced therapeutic efficacy of compound of formula I compared to the chemically related compounds galunisertib and reference compound 338, which is, in fact, a conformational isomer of the compound of formula (I).

### Example 9: In Vivo Combination Therapy Assays with PD1/PDL1 Checkpoint Immunotherapy

In vivo studies were carried out to evaluate the activity on mice with overt metastatic disease of the compound of formula I versus reference compounds galunisertib of the prior art, alone and in combination with PD1/PDL1 immune checkpoint inhibitor such as anti-PD-1 antibody treatment.

Mouse tumor organoids (MTO129) were developed as indicated in example 3. C57BL/6J mice were purchased from Janvier at 6 weeks of age and injected at 7-8 weeks. For transplantation of MTO129 into the liver, cells were trypsinized (digesting the matrix and disaggregating organoids into single cells), counted and 300,000 cells were injected in HBSS (Hank's Balanced Salt Solution, Lonza) into the spleen, thus delivering the cells directly into the portal vein that connects the intestine to the liver. Compounds (compound of formula I or galunisertib) were prepared as suspensions in a vehicle solution composed of 1% sodium carboxymethylcellulose (Sigma), 0.4% sodium dodecyl sulfate (Sigma), 0.085% polyvinylpyrrolidone, 0.05% antifoam-A (Sigma) in milli-Q water. Treatment with compounds occurred by gavage (tube feeding) and mice were treated twice a day with 150 μl of compound suspension in vehicle. Mice were treated with 1× equimolar doses of each compound that correspond to 80 mg/k b.i.d of galunisertib or 70.8 mg/kg b.i.d of the compound of formula I. 200 g of anti-PD1 (clone RMPI-14; Leinco Technologies) was given every 3 days by intraperitoneal injection. Control mice were given gavage vehicle and IgG2a isotype control antibody.

In experiments shown in FIG. 14 and FIG. 16, treatment with compounds and the indicated antibodies started at day 16 post-inoculation of MTO129. At this time point, mice had developed large liver metastases. Treatments finished at day 24. FIG. 14 shows liver tumor count (LiMs) after treatment with compound of formula I alone compared to prior art compound galunisertib, both at 1× equimolar doses (80 mg/k b.i.d of galunisertib or 70.8 mg/kg b.i.d of the compound of formula I) as well as after treatment with anti-PD-1 antibodies, or a combination treatment with anti-PD-1 antibodies plus compound of formula I or galunisertib. Again, the results show that the compound of formula I provides an improved activity at equimolar doses as monotherapy. In addition, the combination therapy of anti-PD-1 antibodies and the compound of formula I provided an improved activity than that of the combination of the anti-PD-1 antibodies with galunisertib. Remarkably, the majority of mice treated with compound of formula I in combination with PD-1 antibody therapy remained metastasis free at experimental time points. FIG. 16 shows survival of mice with overt metastatic disease after treatment has finalized. In these experiments, the vast majority (>90%) of control mice die of liver metastatic disease. In contrast, 80% of mice treated with compound in formula I in combination with anti-PD1 antibody survived after treatment. Mice treated with the compound of formula I combined with anti-PD1 antibodies had much higher survival rates than mice treated with galunisertib plus anti-PD1 antibodies (80% versus 20%).

### Example 10: In Vivo Therapeutic Efficacy of Compound of Formula I and Reference Compounds 338 and 337

In another experiment (FIG. 17), we compared the in vivo therapeutic activity of compound of formula I to the related compound 338 and compound 337. Mouse tumor organoids (MTO129) were developed as indicated in example 3. C57BL/6J mice were purchased from Janvier at 6 weeks of age and injected at 7-8 weeks. For transplantation of MTO129 into the liver, cells were trypsinized (digesting the matrix and disaggregating organoids into single cells), counted and 200,000 cells were injected in HBSS (Hank's Balanced Salt Solution, Lonza) into the spleen, thus delivering the cells directly into the portal vein that connects the intestine to the liver. Mice were treated with 1× equivalent molar doses of each compound (compound of formula I at 70.8 mg/kg b.i.d., of compound 338 at 70.8 mg/kg b.i.d. or of compound 337 at 68.0 mg/kg b.i.d.). All mice were treated from day 2 to day 18 post inoculation of tumor cells. Number of liver metastases were counted at 21 days after MTO inoculation. The experiment (FIG. 17) evidenced that compound of formula I holds enhanced potency to block the formation of metastasis compared to compound 338 and compound 337.

1. A compound of formula (I), or a pharmaceutical salt, or a pharmaceutically acceptable solvate thereof:

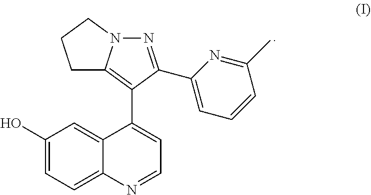 [SMILES: Cc1cccc(-c2nn3c(c2-c2ccnc4ccc(O)cc24)CCC3)n1]

2. (canceled)

3. A method for treating a disease responsive to TGFβ signaling inhibition, wherein said method comprises administering an effective amount of a compound of claim 1 to a subject in need thereof.

4. The method according to claim 3, wherein said compound is administered in combination with at least one of chemotherapy, a cancer targeted therapy, immunotherapy and/or an immune checkpoint inhibitor.

5. (canceled)

6. (canceled)

7. The method according to claim 3, wherein the disease responsive to TGFβ signal inhibition is selected from the group consisting of cancer, scleroderma, psoriasis, anemia, sarcopenia, Alzheimer's disease, Marfan syndrome, aneurysm, pulmonary hypertension, osteogenesis imperfecta, idiopathic pulmonary fibrosis, liver fibrosis, cirrhosis, hepatic steatosis, hypertrophic cardiomyopathy, myelofibrosis, neurofibromatosis type I, fibrotic kidney disease, focal segmental glomerulosclerosis, radiation-induced fibrosis, skin fibrosis in systemic sclerosis, diffuse systemic sclerosis, scarring, corneal primary pterygium, fibrosis, uterine leiomyoma, obesity, diabetes, microangiopathy in diabetic retinopathy and nephropathy.

8. The method according to claim 3, wherein the disease responsive to TGFβ signal inhibition is a cancer selected from the group consisting of hematologic cancer; B-cell or T-cell leukemia, non-Hodgkin lymphoma, non-Hodgkin lymphoma B-cell or T-cell types, Burkitt lymphoma, Hodgkin lymphoma, leukemias, lymphoma B-cell or T-cell types, multiple myeloma, brain cancer, cancer of glial lineage of the central nervous system (glioma), sarcomas, fibrosarcoma, malignant fibrous histiocytoma, Ewing's sarcoma, osteosarcoma, malignant pleural mesothelioma, breast cancer, breast cancer resistant to anti-HER2 therapy, breast carcinoma, breast adenocarcinoma, gastric and gastroesophageal cancer, gastric carcinoma, gastric adenocarcinoma, colorectal cancer, colon carcinoma, colon adenocarcinoma, rectum cancer, colorectal carcinoma, metastatic colon cancer, pancreatic cancer, pancreas carcinoma, pancreatic adenocarcinoma, renal cell carcinoma, clear cell renal cell carcinoma, liver cancer, liver metastatic cancer, metastatic disease, lung cancer, lung carcinoma, lung adenocarcinoma, non-small-cell lung cancer, small-cell lung cancer, ovarian cancer, ovarian carcinoma, ovarian adenocarcinoma, ovarian carcinoma, endometrial carcinoma, endometrial stromal sarcoma, carcinoma of the uterine cervix, thyroid carcinoma, metastasizing papillary thyroid carcinoma, follicular thyroid carcinoma, bladder carcinoma, urine bladder carcinoma, transitional cell carcinoma of the urinary bladder, prostate cancer, prostate carcinoma, neuroendocrine cancers, squamous cell carcinoma, osteosarcoma, rhabdomyosarcoma, embryonal cancers, neuroblastoma, medulloblastoma, retinoblastoma, nephroblastoma, hepatoblastoma, melanoma and skin cancer.

9. The method according to claim 8, wherein the cancer is a metastatic cancer.

10. A method for preventing cancer metastasis wherein said method comprises administering an effective amount of a compound according to claim 1 to a subject in need thereof.

11. A pharmaceutical composition comprising an effective amount of a compound according to claim 1 and at least one pharmaceutical acceptable excipient or carrier.

12. A pharmaceutical composition according to claim 11, further comprising at least another active ingredient, wherein said at least another active ingredient is an immunotherapeutic agent or a chemotherapeutic agent.

13. (canceled)

14. A prodrug of the compound of claim 1
wherein said prodrug is selected from:
a compound of formula (II), or a pharmaceutically acceptable salt, or a pharmaceutically acceptable solvate thereof: 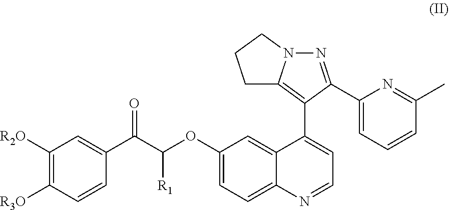
wherein
R₁ is selected from the group consisting of H, alkyl, cycloalkyl, haloalkyl, hydroxyalkyl, aminoalkyl, alkanoylamino, alkoxy, aryl, alkylaryl, arylalkyl, amino, N-alkylamino and N,N-dialkylamino; and
R₂ and R₃ are each independently selected from the group consisting of H, alkyl and haloalkyl; or
a compound of formula (IV), or a pharmaceutically acceptable salt, or a pharmaceutically acceptable solvate thereof: 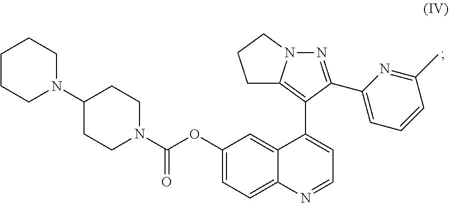 [SMILES: Cc1cccc(-c2nn3c(c2-c2ccnc4ccc(OC(=O)N5CCC(N6CCCCC6)CC5)cc24)CCC3)n1]
and wherein said prodrug releases the compound of formula I when metabolized.

15. The prodrug according to claim 14 wherein said prodrug has formula III, or a pharmaceutically acceptable salt or a pharmaceutically acceptable solvate thereof:

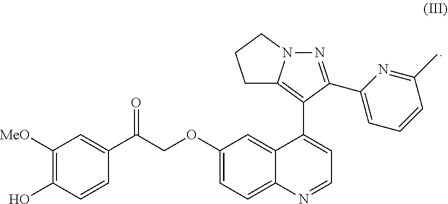 [SMILES: COc1cc(C(=O)COc2ccc3nccc(-c4c(-c5cccc(C)n5)nn5c4CCC5)c3c2)ccc1O]

16. (canceled)

17. (canceled)

18. A method for preventing and or treating a disease responsive to inhibitors of the TGFβ pathway, wherein said method comprises administering an effective amount of a prodrug of the compound according to claim 14 to a subject in need thereof.

19. The method according to claim 18, wherein said prodrug of the compound of formula (I) is administered in combination with at least one of chemotherapy, a cancer targeted therapy, immunotherapy, and/or immune checkpoint inhibitors.

20. (canceled)

21. (canceled)

22. The method according to claim 18, wherein the disease is selected from the group consisting of cancer, scleroderma, psoriasis, anemia, sarcopenia, Alzheimer's disease, Marfan syndrome, aneurysm, pulmonary hypertension, osteogenesis imperfecta, idiopathic pulmonary fibrosis, liver fibrosis, cirrhosis, hepatic steatosis, hypertrophic cardiomyopathy, myelofibrosis, neurofibromatosis type I, fibrotic kidney disease, focal segmental glomerulosclerosis, radiation-induced fibrosis, uterine leiomyoma, obesity, diabetes, microangiopathy in diabetic retinopathy and nephropathy

23. The method according to claim 22, wherein the disease is a cancer selected from the group consisting of hematologic cancer; B-cell or T-cell leukemia, non-Hodgkin lymphoma, non-Hodgkin lymphoma B-cell or T-cell types, Burkitt lymphoma, Hodgkin lymphoma, leukemias, lymphoma B-cell or T-cell types, multiple myeloma, brain cancer, cancer of glial lineage of the central nervous system (glioma), sarcomas, fibrosarcoma, malignant fibrous histiocytoma, Ewing's sarcoma, osteosarcoma, malignant pleural mesothelioma, breast cancer, breast cancer resistant to anti-HER2 therapy, breast carcinoma, breast adenocarcinoma, gastric and gastroesophageal cancer, gastric carcinoma, gastric adenocarcinoma, colorectal cancer, colon carcinoma, colon adenocarcinoma, rectum cancer, colorectal carcinoma, metastatic colon cancer, pancreatic cancer, pancreas carcinoma, pancreatic adenocarcinoma, renal cell carcinoma, clear cell renal cell carcinoma, liver cancer, liver metastatic cancer, metastatic disease, lung cancer, lung carcinoma, lung adenocarcinoma, non-small-cell lung cancer, small-cell lung cancer, ovarian cancer, ovarian carcinoma, ovarian adenocarcinoma, ovarian carcinoma, endometrial carcinoma, endometrial stromal sarcoma, carcinoma of the uterine cervix, thyroid carcinoma, metastasizing papillary thyroid carcinoma, follicular thyroid carcinoma, bladder carcinoma, urine bladder carcinoma, transitional cell carcinoma of the urinary bladder, prostate cancer, prostate carcinoma, neuroendocrine cancers, squamous cell carcinoma, osteosarcoma, rhabdomyosarcoma, embryonal cancers, neuroblastoma, medulloblastoma, retinoblastoma, nephroblastoma, hepatoblastoma, melanoma and skin cancer.

24. The method according to claim 23, wherein the cancer is a metastatic cancer.

25. A method for preventing cancer metastasis wherein said method comprises administering an effective amount of a prodrug according to claim 14 to a subject in need thereof.

26. A pharmaceutical composition comprising an effective amount of a prodrug according to claim 14, or a pharmaceutically acceptable salt or solvate thereof, and at least one pharmaceutical acceptable excipient or carrier.

27. The pharmaceutical composition according to claim 26, further comprising at least another active ingredient, wherein said at least another active ingredient is an immunotherapeutic agent or a chemotherapeutic agent.

28. (canceled)
